SEQUENCE LISTING (176 sequences)

SEQ ID NO: 1 - protein, 10 aa, Artificial Sequence
  ACETYLATION (1)...(1)
  AMIDATION (10)...(10): chemically synthesized peptide
YEFRDRMRTH

SEQ ID NO: 2 - protein, 23 aa, Artificial Sequence
  ACETYLATION (1)...(1)
  AMIDATION (23)...(23): chemically synthesized peptide
PVAEEFRDRMRTHVDSLRTQLAP

SEQ ID NO: 3 - protein, 20 aa, Artificial Sequence
  ACETYLATION (1)...(1)
  AMIDATION (20)...(20): chemically synthesized peptide
EEFRDRMRTHVDSLRTQLAP

SEQ ID NO: 4 - protein, 18 aa, Artificial Sequence
  ACETYLATION (1)...(1)
  AMIDATION (18)...(18): chemically synthesized peptide
FRDRMRTHVDSLRTQLAP

SEQ ID NO: 5 - protein, 17 aa, Artificial Sequence
  ACETYLATION (1)...(1)
  AMIDATION (17)...(17): chemically synthesized peptide
RDRMRTHVDSLRTQLAP

SEQ ID NO: 6 - protein, 13 aa, Artificial Sequence
  ACETYLATION (1)...(1)
  AMIDATION (13)...(13): chemically synthesized peptide
RTHVDSLRTQLAP

SEQ ID NO: 7 - protein, 12 aa, Artificial Sequence
  ACETYLATION (1)...(1)
  AMIDATION (12)...(12): chemically synthesized peptide
THVDSLRTQLAP

SEQ ID NO: 8 - protein, 9 aa, Artificial Sequence
  ACETYLATION (1)...(1)
  AMIDATION (9)...(9): chemically synthesized peptide
DSLRTQLAP

SEQ ID NO: 9 - protein, 14 aa, Artificial Sequence
  ACETYLATION (1)...(1)
  AMIDATION (14)...(14): chemically synthesized peptide
PVAEEFRDRMRTHV

SEQ ID NO: 10 - protein, 11 aa, Artificial Sequence
  ACETYLATION (1)...(1)
  AMIDATION (11)...(11): chemically synthesized peptide
PVAEEFRDRMR

SEQ ID NO: 11 - protein, 10 aa, Artificial Sequence
  ACETYLATION (1)...(1)
  AMIDATION (10)...(10): chemically synthesized peptide
PVAEEFRDRM

SEQ ID NO: 12 - protein, 9 aa, Artificial Sequence
  ACETYLATION (1)...(1)
  AMIDATION (9)...(9): chemically synthesized peptide
PVAEEFRDR

SEQ ID NO: 13 - protein, 6 aa, Artificial Sequence
  ACETYLATION (1)...(1)
  AMIDATION (6)...(6): chemically synthesized peptide
PVAEEF

SEQ ID NO: 14 - protein, 5 aa, Artificial Sequence
  ACETYLATION (1)...(1)
  AMIDATION (5)...(5): chemically synthesized peptide
PVAEE

SEQ ID NO: 15 - protein, 14 aa, Artificial Sequence
  ACETYLATION (1)...(1)
  AMIDATION (14)...(14): chemically synthesized peptide
MRTHVDSLRTQLAP

SEQ ID NO: 16 - protein, 18 aa, Artificial Sequence
  ACETYLATION (1)...(1)
  AMIDATION (18)...(18): chemically synthesized peptide
PVAEEFRDRMRTHVDSLR

SEQ ID NO: 17 - protein, 7 aa, Artificial Sequence
  ACETYLATION (1)...(1)
  AMIDATION (7)...(7): chemically synthesized peptide
WDKVKDF

SEQ ID NO: 18 - protein, 11 aa, Artificial Sequence
  ACETYLATION (1)...(1)
  AMIDATION (11)...(11): chemically synthesized peptide
SGRDYVSQFES

SEQ ID NO: 19 - protein, 11 aa, Artificial Sequence
  ACETYLATION (1)...(1)
  AMIDATION (11)...(11): chemically synthesized peptide
YLDEFQKKWKE

SEQ ID NO: 20 - protein, 14 aa, Artificial Sequence
  ACETYLATION (1)...(1)
  AMIDATION (14)...(14): chemically synthesized peptide
TRDFWDNLEKETDW

SEQ ID NO: 21 - protein, 17 aa, Artificial Sequence
  ACETYLATION (1)...(1)
  AMIDATION (17)...(17): chemically synthesized peptide
WDKVKDFANVYVDAVKD

SEQ ID NO: 22 - protein, 10 aa, Artificial Sequence
  MOD_RES (1)...(1): N terminus is phenylacetylated
YEFRDRMRTH

SEQ ID NO: 23 - protein, 10 aa, Artificial Sequence
  ACETYLATION (1)...(1)
  AMIDATION (10)...(10): chemically synthesized peptide
YEFRDRMRTH

SEQ ID NO: 24 - protein, 10 aa, Artificial Sequence
  chemically synthesized peptide
YEFRDRMRTH

SEQ ID NO: 25 - protein, 9 aa, Artificial Sequence
  chemically synthesized peptide
EFRDRMRTH

SEQ ID NO: 26 - protein, 7 aa, Artificial Sequence
  chemically synthesized peptide
EFRDRMR

SEQ ID NO: 27 - protein, 8 aa, Artificial Sequence
  chemically synthesized peptide
FRDRMRTH

SEQ ID NO: 28 - protein, 9 aa, Artificial Sequence
  chemically synthesized peptide
YFRDRMRTH

SEQ ID NO: 29 - protein, 8 aa, Artificial Sequence
  chemically synthesized peptide
EEFRDRMR

SEQ ID NO: 30 - protein, 8 aa, Artificial Sequence
  chemically synthesized peptide
FRDRFRDR

SEQ ID NO: 31 - protein, 6 aa, Artificial Sequence
  chemically synthesized peptide
EFRDRM

SEQ ID NO: 32 - protein, 5 aa, Artificial Sequence
  chemically synthesized peptide
EFRDR

SEQ ID NO: 33 - protein, 4 aa, Artificial Sequence
  chemically synthesized peptide
EFRD

SEQ ID NO: 34 - protein, 3 aa, Artificial Sequence
  chemically synthesized peptide
EFR

SEQ ID NO: 35 - protein, 7 aa, Artificial Sequence
  chemically synthesized peptide
RDRMRTH

SEQ ID NO: 36 - protein, 6 aa, Artificial Sequence
  chemically synthesized peptide
DRMRTH

SEQ ID NO: 37 - protein, 7 aa, Artificial Sequence
  chemically synthesized peptide
FRDRMRT

SEQ ID NO: 38 - protein, 6 aa, Artificial Sequence
  chemically synthesized peptide
FRDRMR

SEQ ID NO: 39 - protein, 7 aa, Artificial Sequence
  chemically synthesized peptide
FFRDRMR

SEQ ID NO: 40 - protein, 5 aa, Artificial Sequence
  chemically synthesized peptide
FRDRM

SEQ ID NO: 41 - protein, 6 aa, Artificial Sequence
  chemically synthesized peptide
YFRDRM

SEQ ID NO: 42 - protein, 5 aa, Artificial Sequence
  chemically synthesized peptide
YFRDR

SEQ ID NO: 43 - protein, 5 aa, Artificial Sequence
  chemically synthesized peptide
FRDRF

SEQ ID NO: 44 - protein, 9 aa, Artificial Sequence
  chemically synthesized peptide
EFRDRMRTF

SEQ ID NO: 45 - protein, 6 aa, Artificial Sequence
  chemically synthesized peptide
EFRDRF

SEQ ID NO: 46 - protein, 6 aa, Artificial Sequence
  chemically synthesized peptide
FRDRFF

SEQ ID NO: 47 - protein, 6 aa, Artificial Sequence
  chemically synthesized peptide
FRDRFY

SEQ ID NO: 48 - protein, 9 aa, Artificial Sequence
  chemically synthesized peptide
FFDRFRDRF

SEQ ID NO: 49 - protein, 9 aa, Artificial Sequence
  chemically synthesized peptide
MRDRFRDRM

SEQ ID NO: 50 - protein, 9 aa, Artificial Sequence
  chemically synthesized peptide
FRDRFRDRF

SEQ ID NO: 51 - protein, 9 aa, Artificial Sequence
  chemically synthesized peptide
FRDRMRDRM

SEQ ID NO: 52 - protein, 9 aa, Artificial Sequence
  chemically synthesized peptide
MRDRFRDRM

SEQ ID NO: 53 - protein, 7 aa, Artificial Sequence
  chemically synthesized peptide
RMRDRMR

SEQ ID NO: 54 - protein, 9 aa, Artificial Sequence
  chemically synthesized peptide
FRDRMRDRF

SEQ ID NO: 55 - protein, 11 aa, Artificial Sequence
  chemically synthesized peptide
EFRDRMRDRFE

SEQ ID NO: 56 - protein, 4 aa, Artificial Sequence
  chemically synthesized peptide
FRDR

SEQ ID NO: 57 - protein, 3 aa, Artificial Sequence
  chemically synthesized peptide
FRD

SEQ ID NO: 58 - protein, 4 aa, Artificial Sequence
  chemically synthesized peptide
YFRD

SEQ ID NO: 59 - protein, 9 aa, Artificial Sequence
  chemically synthesized peptide
FRDRMRDRM

SEQ ID NO: 60 - protein, 5 aa, Artificial Sequence
  chemically synthesized peptide
MRDRM

SEQ ID NO: 61 - protein, 5 aa, Artificial Sequence
  chemically synthesized peptide
MRDRM

SEQ ID NO: 62 - protein, 5 aa, Artificial Sequence
  chemically synthesized peptide
FRDRF

SEQ ID NO: 63 - protein, 5 aa, Artificial Sequence
  chemically synthesized peptide
FRDRF

SEQ ID NO: 64 - protein, 7 aa, Artificial Sequence
  chemically synthesized peptide
RMRDRMR

SEQ ID NO: 65 - protein, 5 aa, Artificial Sequence
  chemically synthesized peptide
DRMRD

SEQ ID NO: 66 - protein, 5 aa, Artificial Sequence
  chemically synthesized peptide
DRMRD

SEQ ID NO: 67 - protein, 9 aa, Artificial Sequence
  chemically synthesized peptide
FRDRMRDRF

SEQ ID NO: 68 - protein, 6 aa, Artificial Sequence
  chemically synthesized peptide
RFEEFR

SEQ ID NO: 69 - protein, 5 aa, Artificial Sequence
  chemically synthesized peptide
FRTRF

SEQ ID NO: 70 - protein, 5 aa, Artificial Sequence
  chemically synthesized peptide
FRMRF

SEQ ID NO: 71 - protein, 10 aa, Artificial Sequence
  chemically synthesized peptide
EFRDRMRDRF

SEQ ID NO: 72 - protein, 6 aa, Artificial Sequence
  chemically synthesized peptide
DRMRDF

SEQ ID NO: 73 - protein, 13 aa, Artificial Sequence
  chemically synthesized peptide
YYYPEFRDRMRTH

SEQ ID NO: 74 - protein, 11 aa, Artificial Sequence
  chemically synthesized peptide
YYPEFRDRMRT

SEQ ID NO: 75 - protein, 12 aa, Artificial Sequence
  chemically synthesized peptide
YYYPEFRDRMRT

SEQ ID NO: 76 - protein, 8 aa, Artificial Sequence
  chemically synthesized peptide
EFRDRMRY

SEQ ID NO: 77 - protein, 11 aa, Artificial Sequence
  chemically synthesized peptide
YYYPEFRDRMR

SEQ ID NO: 78 - protein, 7 aa, Artificial Sequence
  chemically synthesized peptide
YEFRDRM

SEQ ID NO: 79 - protein, 10 aa, Artificial Sequence
  chemically synthesized peptide
YYYPEFRDRM

SEQ ID NO: 80 - protein, 6 aa, Artificial Sequence
  chemically synthesized peptide
EFRDRY

SEQ ID NO: 81 - protein, 5 aa, Artificial Sequence
  chemically synthesized peptide
EFRDY

SEQ ID NO: 82 - protein, 8 aa, Artificial Sequence
  chemically synthesized peptide
YYYPEFRD

SEQ ID NO: 83 - protein, 4 aa, Artificial Sequence
  chemically synthesized peptide
EFRY

SEQ ID NO: 84 - protein, 6 aa, Artificial Sequence
  chemically synthesized peptide
YYPEFR

SEQ ID NO: 85 - protein, 9 aa, Artificial Sequence
  chemically synthesized peptide
YYYPEFRDR

SEQ ID NO: 86 - protein, 3 aa, Artificial Sequence
  chemically synthesized peptide
ERF

SEQ ID NO: 87 - protein, 6 aa, Artificial Sequence
  chemically synthesized peptide
EEFRDR

SEQ ID NO: 88 - protein, 3 aa, Artificial Sequence
  chemically synthesized peptide
EYR

SEQ ID NO: 89 - protein, 3 aa, Artificial Sequence
  chemically synthesized peptide
EFR

SEQ ID NO: 90 - protein, 3 aa, Artificial Sequence
  chemically synthesized peptide
EFR

SEQ ID NO: 91 - protein, 3 aa, Artificial Sequence
  chemically synthesized peptide
EFR

SEQ ID NO: 92 - protein, 3 aa, Artificial Sequence
  chemically synthesized peptide
EFR

SEQ ID NO: 93 - protein, 3 aa, Artificial Sequence
  chemically synthesized peptide
EFR

SEQ ID NO: 94 - protein, 3 aa, Artificial Sequence
  chemically synthesized peptide
EFR

SEQ ID NO: 95 - protein, 3 aa, Artificial Sequence
  chemically synthesized peptide
RFE

SEQ ID NO: 96 - protein, 3 aa, Artificial Sequence
  chemically synthesized peptide
EFR

SEQ ID NO: 97 - protein, 3 aa, Artificial Sequence
  chemically synthesized peptide
EFR

SEQ ID NO: 98 - protein, 4 aa, Artificial Sequence
  chemically synthesized peptide
YEFR

SEQ ID NO: 99 - protein, 4 aa, Artificial Sequence
  chemically synthesized peptide
EFRY

SEQ ID NO: 100 - protein, 4 aa, Artificial Sequence
  chemically synthesized peptide
EFRY

SEQ ID NO: 101 - protein, 4 aa, Artificial Sequence
  chemically synthesized peptide
EFRY

SEQ ID NO: 102 - protein, 3 aa, Artificial Sequence
  chemically synthesized peptide
ERF

SEQ ID NO: 103 - protein, 4 aa, Artificial Sequence
  chemically synthesized peptide
YEFR

SEQ ID NO: 104 - protein, 3 aa, Artificial Sequence
  chemically synthesized peptide
EFR

SEQ ID NO: 105 - protein, 3 aa, Artificial Sequence
  chemically synthesized peptide
EFR

SEQ ID NO: 106 - protein, 5 aa, Artificial Sequence
  chemically synthesized peptide
FEEFR

SEQ ID NO: 107 - protein, 9 aa, Artificial Sequence
  chemically synthesized peptide
YWHVWQQDE

SEQ ID NO: 108 - protein, 9 aa, Artificial Sequence
  chemically synthesized peptide
YQWDKVKDF

SEQ ID NO: 109 - protein, 9 aa, Artificial Sequence
  chemically synthesized peptide
ENWDTLGSY

SEQ ID NO: 110 - protein, 11 aa, Artificial Sequence
  chemically synthesized peptide
SGRDYVSQFES

SEQ ID NO: 111 - protein, 17 aa, Artificial Sequence
  chemically synthesized peptide
VRQEMNKDLEEVKQKVY

SEQ ID NO: 112 - protein, 12 aa, Artificial Sequence
  chemically synthesized peptide
YQMRESLAQRLY

SEQ ID NO: 113 - protein, 15 aa, Artificial Sequence
  chemically synthesized peptide
TRDFWDNLEKETDWY

SEQ ID NO: 114 - protein, 10 aa, Artificial Sequence
  chemically synthesized peptide
DEFQKKWKEY

SEQ ID NO: 115 - protein, 12 aa, Artificial Sequence
  chemically synthesized peptide
WKEDVELYRQKV

SEQ ID NO: 116 - protein, 13 aa, Artificial Sequence
  chemically synthesized peptide
YSLAQRLAELKSY

SEQ ID NO: 117 - protein, 13 aa, Artificial Sequence
  chemically synthesized peptide
QESARQKLQELQY

SEQ ID NO: 118 - protein, 4 aa, Artificial Sequence
  chemically synthesized peptide
YERF

SEQ ID NO: 119 - protein, 3 aa, Artificial Sequence
  chemically synthesized peptide
RFE

SEQ ID NO: 120 - protein, 3 aa, Artificial Sequence
  chemically synthesized peptide
EFR

SEQ ID NO: 121 - protein, 4 aa, Artificial Sequence
  chemically synthesized peptide
YERF

SEQ ID NO: 122 - protein, 3 aa, Artificial Sequence
  chemically synthesized peptide
EFR

SEQ ID NO: 123 - protein, 4 aa, Artificial Sequence
  chemically synthesized peptide
ERFY

SEQ ID NO: 124 - protein, 4 aa, Artificial Sequence
  chemically synthesized peptide
ERFY

SEQ ID NO: 125 - protein, 3 aa, Artificial Sequence
  chemically synthesized peptide
EFR

SEQ ID NO: 126 - protein, 3 aa, Artificial Sequence
  chemically synthesized peptide
EFR

SEQ ID NO: 127 - protein, 1 aa, Artificial Sequence
  chemically synthesized peptide
F

SEQ ID NO: 128 - protein, 3 aa, Artificial Sequence
  chemically synthesized peptide
EFR

SEQ ID NO: 129 - protein, 1 aa, Artificial Sequence
  chemically synthesized peptide
A

SEQ ID NO: 130 - protein, 1 aa, Artificial Sequence
  chemically synthesized peptide
A

SEQ ID NO: 131 - protein, 1 aa, Artificial Sequence
  chemically synthesized peptide
A

SEQ ID NO: 132 - protein, 1 aa, Artificial Sequence
  chemically synthesized peptide
A

SEQ ID NO: 133 - protein, 2 aa, Artificial Sequence
  chemically synthesized peptide
FA

SEQ ID NO: 134 - protein, 2 aa, Artificial Sequence
  chemically synthesized peptide
FA

SEQ ID NO: 135 - protein, 3 aa, Artificial Sequence
  chemically synthesized peptide
FRE

SEQ ID NO: 136 - protein, 4 aa, Artificial Sequence
  chemically synthesized peptide
ERFY

SEQ ID NO: 137 - protein, 3 aa, Artificial Sequence
  chemically synthesized peptide
EFR

SEQ ID NO: 138 - protein, 4 aa, Artificial Sequence
  chemically synthesized peptide
YFRE

SEQ ID NO: 139 - protein, 4 aa, Artificial Sequence
  chemically synthesized peptide
YFRE

SEQ ID NO: 140 - protein, 3 aa, Artificial Sequence
  chemically synthesized peptide
ERF

SEQ ID NO: 141 - protein, 4 aa, Artificial Sequence
  chemically synthesized peptide
EFRY

SEQ ID NO: 142 - protein, 4 aa, Artificial Sequence
  chemically synthesized peptide
FREY

SEQ ID NO: 143 - protein, 4 aa, Artificial Sequence
  chemically synthesized peptide
FREY

SEQ ID NO: 144 - protein, 2 aa, Artificial Sequence
  chemically synthesized peptide
FR

SEQ ID NO: 145 - protein, 3 aa, Artificial Sequence
  chemically synthesized peptide
EFR

SEQ ID NO: 146 - protein, 3 aa, Artificial Sequence
  chemically synthesized peptide
EFR

SEQ ID NO: 147 - protein, 3 aa, Artificial Sequence
  chemically synthesized peptide
EFR

SEQ ID NO: 148 - protein, 4 aa, Artificial Sequence
  chemically synthesized peptide
RFEY

SEQ ID NO: 149 - protein, 4 aa, Artificial Sequence
  chemically synthesized peptide
FREY

SEQ ID NO: 150 - protein, 3 aa, Artificial Sequence
  chemically synthesized peptide
EFR

SEQ ID NO: 151 - protein, 3 aa, Artificial Sequence
  chemically synthesized peptide
ERF

SEQ ID NO: 152 - protein, 4 aa, Artificial Sequence
  chemically synthesized peptide
FREY

SEQ ID NO: 153 - protein, 4 aa, Artificial Sequence
  chemically synthesized peptide
RFEY

SEQ ID NO: 154 - protein, 3 aa, Artificial Sequence
  chemically synthesized peptide
FER

SEQ ID NO: 155 - protein, 4 aa, Artificial Sequence
  chemically synthesized peptide
REFY

SEQ ID NO: 156 - protein, 4 aa, Artificial Sequence
  chemically synthesized peptide
REFY

SEQ ID NO: 157 - protein, 4 aa, Artificial Sequence
  chemically synthesized peptide
REFY

SEQ ID NO: 158 - protein, 4 aa, Artificial Sequence
  chemically synthesized peptide
REFY

SEQ ID NO: 159 - protein, 3 aa, Artificial Sequence
  chemically synthesized peptide
FER

SEQ ID NO: 160 - protein, 4 aa, Artificial Sequence
  chemically synthesized peptide
YREF

SEQ ID NO: 161 - protein, 4 aa, Artificial Sequence
  chemically synthesized peptide
YREF

SEQ ID NO: 162 - protein, 4 aa, Artificial Sequence
  chemically synthesized peptide
FERY

SEQ ID NO: 163 - protein, 2 aa, Artificial Sequence
  chemically synthesized peptide
FR

SEQ ID NO: 164 - protein, 4 aa, Artificial Sequence
  chemically synthesized peptide
FERY

SEQ ID NO: 165 - protein, 4 aa, Artificial Sequence
  chemically synthesized peptide
RFEY

SEQ ID NO: 166 - protein, 4 aa, Artificial Sequence
  chemically synthesized peptide
FERY

SEQ ID NO: 167 - protein, 2 aa, Artificial Sequence
  chemically synthesized peptide
RF

SEQ ID NO: 168 - protein, 4 aa, Artificial Sequence
  chemically synthesized peptide
YFER

SEQ ID NO: 169 - protein, 4 aa, Artificial Sequence
  chemically synthesized peptide
YFER

SEQ ID NO: 170 - protein, 3 aa, Artificial Sequence
  chemically synthesized peptide
FER

SEQ ID NO: 171 - protein, 4 aa, Artificial Sequence
  chemically synthesized peptide
YFER

SEQ ID NO: 172 - protein, 3 aa, Artificial Sequence
  chemically synthesized peptide
RFE

SEQ ID NO: 173 - protein, 4 aa, Artificial Sequence
  chemically synthesized peptide
YFER

SEQ ID NO: 174 - protein, 3 aa, Artificial Sequence
  chemically synthesized peptide
RFE

SEQ ID NO: 175 - protein, 3 aa, Artificial Sequence
  chemically synthesized peptide
RFE

SEQ ID NO: 176 - protein, 3 aa, Artificial Sequence
  chemically synthesized peptide
EFR

(19) United States
(12) Patent Application Publication
(10) Pub. No.: US 2006/0166891 A1
(43) Pub. Date: Jul. 27, 2006

(54) Mediators of reverse cholesterol transport for the treatment of hypercholesterolemia

(76) Inventors: Jagadish C. Sircar, San Diego, CA (US); Kashinatham Alisala, San Diego, CA (US); Igor Nikoulin, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/829,855

(22) Filed: Apr. 22, 2004

Related U.S. Application Data
(60) Provisional application No. 60/464,667, filed on Apr. 22, 2003.

(51) Int. Cl. A61K 38/06; A61K 38/08

(52) U.S. Cl. 514/15; 514/16; 514/17; 514/18

(57) ABSTRACT

The present invention provides compositions adapted to enhance reverse cholesterol transport in mammals. The compositions are suitable for oral delivery and useful in the treatment and/or prevention of hypercholesterolemia, atherosclerosis and associated cardiovascular diseases.

### RELATED APPLICATIONS

This application claims priority to U.S. provisional application No. 60/464,667, filed Apr. 22, 2003, which is incorporated herein by reference.

### BACKGROUND OF THE INVENTION

1. Field of the Invention

The invention relates to peptide and small molecule mediators of reverse cholesterol transport (RCT) for treating hypercholesterolemia and associated cardiovascular diseases.

2. Description of the Related Art

It is now well-established that elevated serum cholesterol (“hypercholesterolemia”) is a causal factor in the development of atherosclerosis, a progressive accumulation of cholesterol within the arterial walls. Hypercholesterolemia and atherosclerosis are leading causes of cardiovascular diseases, including hypertension, coronary artery disease, heart attack and stroke. About 1.1 million individuals suffer from heart attack each year in the United States alone, the costs of which are estimated to exceed $117 billion. Although there are numerous pharmaceutical strategies for lowering cholesterol levels in the blood, many of these have undesirable side effests and have raised safety concerns. Moreover, none of the commercially available drug therapies adequately stimulate reverse cholesterol transport, an important metabolic pathway that removes cholesterol from the body.

Circulating cholesterol is carried by plasma lipoproteins—particles of complex lipid and protein composition that transport lipids in the blood. Low density lipoproteins (LDL), and high density lipoproteins (HDL) are the major cholesterol carriers. LDL are believed to be responsible for the delivery of cholesterol from the liver (where it is synthesized or obtained from dietary sources) to extrahepatic tissues in the body. The term “reverse cholesterol transport” describes the transport of cholesterol from extrahepatic tissues to the liver where it is catabolized and eliminated. It is believed that plasma HDL particles play a major role in the reverse transport process, acting as scavengers of tissue cholesterol.

Compelling evidence supports the concept that lipids deposited in atherosclerotic lesions are derived primarily from plasma LDL; thus, LDLs have popularly become known as the “bad” cholesterol. In contrast, plasma HDL levels correlate inversely with coronary heart disease - indeed, high plasma levels of HDL are regarded as a negative risk factor. It is hypothesized that high levels of plasma HDL are not only protective against coronary artery disease, but may actually induce regression of atherosclerotic plaques (e.g. see Badimon et al., 1992, Circulation 86 (Suppl. III):86-94). Thus, HDLs have popularly become known as the “good” cholesterol.

The amount of intracellular cholesterol liberated from the LDLs controls cellular cholesterol metabolism. The accumulation of cellular cholesterol derived from LDLs controls three processes: (1) it reduces cellular cholesterol synthesis by turning off the synthesis of HMGCoA reductase, a key enzyme in the cholesterol biosynthetic pathway; (2) the incoming LDL-derived cholesterol promotes storage of cholesterol by activating LCAT, the cellular enzyme which converts cholesterol into cholesteryl esters that are deposited in storage droplets; and (3) the accumulation of cholesterol within the cell drives a feedback mechanism that inhibits cellular synthesis of new LDL receptors. Cells, therefore, adjust their complement of LDL receptors so that enough cholesterol is brought in to meet their metabolic needs, without overloading. (For a review, see Brown & Goldstein, In: The Pharmacological Basis Of Therapeutics, 8th Ed., Goodman & Gilman, Pergamon Press, NY, 1990, Ch. 36, pp. 874-896).

Reverse cholesterol transport (RCT) is the pathway by which peripheral cell cholesterol can be returned to the liver for recycling to extrahepatic tissues, or excreted into the intestine as bile. The RCT pathway represents the only means of eliminating cholesterol from most extrahepatic tissues. The RCT consists mainly of three steps: (1) cholesterol efflux, the initial removal of cholesterol from peripheral cells; (2) cholesterol esterification by the action of lecithin:cholesterol acyltransferase (LCAT), preventing a re-entry of effluxed cholesterol into the peripheral cells; and (3) uptake/delivery of HDL cholesteryl ester to liver cells. LCAT is the key enzyme in the RCT pathway and is produced mainly in the liver and circulates in plasma associated with the HDL fraction. LCAT converts cell derived cholesterol to cholesteryl esters which are sequestered in HDL destined for removal. The RCT pathway is mediated by HDLs.

HDL is a generic term for lipoprotein particles which are characterized by their high density. The main lipidic constituents of HDL complexes are various phospholipids, cholesterol (ester) and triglycerides. The most prominent apolipoprotein components are A-I and A-II which determine the functional characteristics of HDL.

Each HDL particle contains at least one copy (and usually two to four copies) of apolipoprotein A-1 (ApoA-I). ApoA-I is synthesized by the liver and small intestine as preproapolipoprotein which is secreted as a proprotein that is rapidly cleaved to generate a mature polypeptide having 243 amino acid residues. ApoA-I consists mainly of 6 to 8 different 22 amino acid repeats spaced by a linker moiety which is often proline, and in some cases consists of a stretch made up of several residues. ApoA-I forms three types of stable complexes with lipids: small, lipid-poor complexes referred to as pre-beta-1 HDL; flattened discoidal particles containing polar lipids (phospholipid and cholesterol) referred to as pre-beta-2 HDL; and spherical particles containing both polar and nonpolar lipids, referred to as spherical or mature HDL (HDL₃ and HDL₂). Although most HDL in circulation contains both ApoA-I and ApoA-II, the fraction of HDL which contains only ApoA-I (AI-HDL) appears to be more effective in RCT. Epidemiologic studies support the hypothesis that AI-HDL is anti-atherogenic. (Parra et al., 1992, Arterioscler. Thromb. 12:701-707; Decossin et al., 1997, Eur. J. Clin. Invest. 27:299-307).

Several lines of evidence based on data obtained in vivo implicate the HDL and its major protein component, ApoA-I, in the prevention of atherosclerotic lesions, and potentially, the regression of plaques—making these attractive targets for therapeutic intervention. First, an inverse correlation exists between serum ApoA-I (HDL) concentration and atherogenesis in man (Gordon & Rifkind, 1989, N. Eng. J Med. 321:1311-1316; Gordon et al., 1989, Circulation 79:8-15). Indeed, specific subpopulations of HDL have been associated with a reduced risk for atherosclerosis in humans (Miller, 1987, Amer. Heart 113:589-597; Cheung et al., 1991, Lipid Res. 32:383-394); Fruchart & Ailhaud, 1992, Clin. Chem. 38:79).

Second, animal studies support the protective role of ApoA-I (HDL). Treatment of cholesterol fed rabbits with ApoA-I or HDL reduced the development and progression of plaque (fatty streaks) in cholesterol-fed rabbits (Koizumi et al., 1988, J Lipid Res. 29:1405-1415; Badimon et al., 1989, Lab. Invest. 60:455-461; Badimon et al., 1990, J. Clin. Invest. 85:1234-1241). However, the efficacy varied depending upon the source of HDL (Beitz et al., 1992, Prostaglandins, Leukotrienes and Essential Fatty Acids 47:149-152; Mezdour et al., 1995, Atherosclerosis 113:237-246).

Third, direct evidence for the role of ApoA-I was obtained from experiments involving transgenic animals. The expression of the human gene for ApoA-I transferred to mice genetically predisposed to diet-induced atherosclerosis protected against the development of aortic lesions (Rubin et al., 1991, Nature 353:265-267). The ApoA-I transgene was also shown to suppress atherosclerosis in ApoE-deficient mice and in Apo(a) transgenic mice (Paszty et al., 1994, J. Clin. Invest. 94:899-903; Plump et al., 1994, PNAS. USA 91:9607-9611; Liu et al., 1994, J. Lipid Res. 35:2263-2266). Similar results were observed in transgenic rabbits expressing human ApoA-I (Duverger, 1996, Circulation 94:713-717; Duverger et al., 1996, Arterioscler. Thromb. Vasc. Biol. 16:1424-1429), and in transgenic rats where elevated levels of human ApoA-I protected against atherosclerosis and inhibited restenosis following balloon angioplasty (Burkey et al., 1992, Circulation, Supplement I, 86:1-472, Abstract No. 1876; Burkey et al., 1995, J. Lipid Res. 36:1463-1473).

Current Treatments for Hypercholesterolemia and other Dyslipidemias

In the past two decades or so, the segregation of cholesterolemic compounds into HDL and LDL regulators and recognition of the desirability of decreasing blood levels of LDL has led to the development of a number of drugs. However, many of these drugs have undesirable side effects and/or are contraindicated in certain patients, particularly when administered in combination with other drugs. These drugs and therapeutic strategies include:
(1) bile-acid-binding resins, which interrupt the recycling of bile acids from the intestine to the liver [e.g., cholestyramine (QUESTRAN LIGHT, Bristol-Myers Squibb), and colestipol hydrochloride (COLESTID, Pharmacia & Upjohn Company)];
(2) statins, which inhibit cholesterol synthesis by blocking HMGCoA—the key enzyme involved in cholesterol biosynthesis [e.g., lovastatin (MEVACOR, Merck & Co., Inc.), a natural product derived from a strain of Aspergillus, pravastatin (PRAVACHOL, Bristol-Myers Squibb Co.), and atorvastatin (LIPITOR, Warner Lambert)];
(3) niacin is a water-soluble vitamin B-complex which diminishes production of VLDL and is effective at lowering LDL;
(4) fibrates are used to lower serum triglycerides by reducing the VLDL fraction and may in some patient populations give rise to modest reductions of plasma cholesterol via the same mechanism [e.g., clofibrate (ATROMID-S, Wyeth-Ayerst Laboratories), and gemfibrozil (LOPID, Parke-Davis)];
(5) estrogen replacement therapy may lower cholesterol levels in post-menopausal women;
(6) long chain alpha,omego-dicarboxylic acids have been reported to lower serum triglyceride and cholesterol (See, e.g., Bisgaier et al., 1998, J. Lipid Res. 39:17-30; WO 98/30530; U.S. Pat. No. 4,689,344; WO 99/00116; U.S. Pat. No. 5,756,344; U.S. Pat. No. 3,773,946; U.S. Pat. No. 4,689,344; U.S. Pat. No. 4,689,344; U.S. Pat. No. 4,689,344; and U.S. Pat. No. 3,930,024);
(7) other compounds including ethers (See, e.g., U.S. Pat. No. 4,711,896; U.S. Pat. No. 5,756,544; U.S. Pat. No. 6,506,799), phosphates of dolichol (U.S. Pat. No. 4,613,593), and azolidinedione derivatives (U.S. Pat. No. 4,287,200) are disclosed as lowering serum triglyceride and cholesterol levels.

None of these currently available drugs for lowering cholesterol safely elevate HDL levels and stimulate RCT. Indeed, most of these current treatment strategies appear to operate on the cholesterol transport pathway, modulating dietary intake, recycling, synthesis of cholesterol, and the VLDL population.

ApoA-I Agonists for Treatment of Hypercholesterolemia

In view of the potential role of HDL, i.e., both ApoA-I and its associated phospholipid, in the protection against atherosclerotic disease, human clinical trials utilizing recombinantly produced ApoA-I were commenced, discontinued and apparently re-commenced by UCB Belgium (Pharmaprojects, Oct. 27, 1995; IMS R&D Focus, Jun. 30, 1997; Drug Status Update, 1997, Atherosclerosis 2(6):261-265); see also M. Eriksson at Congress, “The Role of HDL in Disease Prevention,” Nov. 7-9, 1996, Fort Worth; Lacko & Miller, 1997, J. Lip. Res. 38:1267-1273; and WO 94/13819) and were commenced and discontinued by Bio-Tech (Pharmaprojects, Apr. 7, 1989). Trials were also attempted using ApoA-I to treat septic shock (Opal, “Reconstituted HDL as a Treatment Strategy for Sepsis,” IBC's 7th International Conference on Sepsis, Apr. 28-30, 1997, Washington, D.C.; Gouni et al., 1993, J. Lipid Res. 94:139-146; Levine, WO 96/04916). However, there are many pitfalls associated with the production and use of ApoA-I, making it less than ideal as a drug; e.g., ApoA-I is a large protein that is difficult and expensive to produce; significant manufacturing and reproducibility problems must be overcome with respect to stability during storage, delivery of an active product and half-life in vivo.

In view of these drawbacks, attempts have been made to prepare peptides that mimic ApoA-I. Since the key activities of ApoA-I have been attributed to the presence of multiple repeats of a unique secondary structural feature in the protein—a class A amphipathic a-helix (Segrest, 1974, FEBS Lett. 38:247-253; Segrest et al., 1990, PROTEINS: Structure, Function and Genetics 8:103-117), most efforts to design peptides which mimic the activity of ApoA-I have focused on designing peptides which form class A-type amphipathic a-helices (See e.g., Background discussions in U.S. Pat. Nos. 6,376,464 and 6,506,799; incorporated herein in their entirety by reference thereto).

In one study, Fukushima et al. synthesized a 22-residue peptide composed entirely of Glu, Lys and Leu residues arranged periodically so as to form an amphipathic α-helix with equal-hydrophilic and hydrophobic faces (“ELK peptide”) (Fukushima et al., 1979, J. Amer. Chem. Soc. 101(13):3703-3704; Fukushima et al., 1980, J. Biol. Chem. 255:10651-10657). The ELK peptide shares 41% sequence homology with the 198-219 fragment of ApoA-I. The ELK peptide was shown to effectively associate with phospholipids and mimic some of the physical and chemical properties of ApoA-I (Kaiser et al., 1983, PNAS USA 80:1137-1140; Kaiser et al., 1984, Science 223:249-255; Fukushima et al., 1980, supra; Nakagawa et al., 1985, J. Am. Chem. Soc. 107:7087-7092). A dimer of this 22-residue peptide was later found to more closely mimic ApoA-I than the monomer; based on these results, it was suggested that the 44-mer, which is punctuated in the middle by a helix breaker (either Gly or Pro), represented the minimal functional domain in ApoA-I (Nakagawa et al., 1985, supra).

Another study involved model amphipathic peptides called “LAP peptides” (Pownall et al., 1980, PNAS USA 77(6):3154-3158; Sparrow et al., 1981, In: Peptides: Synthesis-Structure-Function, Roch and Gross, Eds., Pierce Chem. Co., Rockford, Ill., 253-256). Based on lipid binding studies with fragments of native apolipoproteins, several LAP peptides were designed, named LAP-1 6, LAP-20 and LAP-24 (containing 16, 20 and 24 amino acid residues, respectively). These model amphipathic peptides share no sequence homology with the apolipoproteins and were designed to have hydrophilic faces organized in a manner unlike the class A-type amphipathic helical domains associated with apolipoproteins (Segrest et al., 1992, J. Lipid Res. 33:141-166). From these studies, the authors concluded that a minimal length of 20 residues is necessary to confer lipid-binding properties to model amphipathic peptides.

Studies with mutants of LAP20 containing a proline residue at different positions in the sequence indicated that a direct relationship exists between lipid binding and LCAT activation, but that the helical potential of a peptide alone does not lead to LCAT activation (Ponsin et al., 1986, J. Biol. Chem. 261(20):9202-9205). Moreover, the presence of this helix breaker (Pro) close to the middle of the peptide reduced its affinity for phospholipid surfaces as well as its ability to activate LCAT. While certain of the LAP peptides were shown to bind phospholipids (Sparrow et al., supra), controversy exists as to the extent to which LAP peptides are helical in the presence of lipids (Buchko et al., 1996, J. Biol. Chem. 271(6):3039-3045; Zhong et al., 1994, Peptide Research 7(2):99-106).

Segrest et al. have synthesized peptides composed of 18 to 24 amino acid residues that share no sequence homology with the helices of ApoA-I (Kannelis et al., 1980, J. Biol. Chem. 255(3):11464-11472; Segrest et al., 1983, J. Biol. Chem. 258:2290-2295). The sequences were specifically designed to mimic the amphipathic helical domains of class A exchangeable apolipoproteins in terms of hydrophobic moment (Eisenberg et al., 1982, Nature 299:371-374) and charge distribution (Segrest et al., 1990, Proteins 8:103-117; U.S. Pat. No. 4,643,988). One 18-residue peptide, the “18A” peptide, was designed to be a model class-A α-helix (Segrest et al., 1990, supra). Studies with these peptides and other peptides having a reversed charged distribution, like the “18R” peptide, have consistently shown that charge distribution is critical for activity; peptides with a reversed charge distribution exhibit decreased lipid affinity relative to the 18A class-A mimics and a lower helical content in the presence of lipids (Kanellis et al., 1980, J. Biol. Chem. 255:11464-11472; Anantharamaiah et al., 1985, J. Biol. Chem. 260:10248-10255; Chung et al., 1985, J. Biol. Chem. 260:10256-10262; Epand et al., 1987, J. Biol. Chem. 262:9389-9396; Anantharamaiah et al., 1991, Adv. Exp. Med. Biol. 285:131-140).

A “consensus” peptide containing 22-amino acid residues based on the sequences of the helices of human ApoA-I has also been designed (Anantharamaiah et al., 1990, Arteriosclerosis 10(1):95-105; Venkatachalapathi et al., 1991, Mol. Conformation and Biol. Interactions, Indian Acad. Sci. B:585-596). The sequence was constructed by identifying the most prevalent residue at each position of the hypothesized helices of human ApoA-I. Like the peptides described above, the helix formed by this peptide has positively charged amino acid residues clustered at the hydrophilic-hydrophobic interface, negatively charged amino acid residues clustered at the center of the hydrophilic face and a hydrophobic angle of less than 180°. While a dimer of this peptide is somewhat effective in activating LCAT, the monomer exhibited poor lipid binding properties (Venkatachalapathi et al., 1991, supra).

Based primarily on in vitro studies with the peptides described above, a set of “rules” has emerged for designing peptides which mimic the function of ApoA-I. Significantly, it is thought that an amphipathic a-helix having positively charged residues clustered at the hydrophilic-hydrophobic interface and negatively charged amino acid residues clustered at the center of the hydrophilic face is required for lipid affinity and LCAT activation (Venkatachalapathi et al., 1991, supra). Anantharamaiah et al. have also indicated that the negatively charged Glu residue at position 13 of the consensus 22-mer peptide, which is positioned within the hydrophobic face of the a-helix, plays an important role in LCAT activation (Anantharamaiah et al., 1991, supra). Furthermore, Brasseur has indicated that a hydrophobic angle (pho angle) of less than 180° is required for optimal lipid-apolipoprotein complex stability, and also accounts for the formation of discoidal particles having the peptides around the edge of the lipid bilayer (Brasseur, 1991, J. Biol. Chem. 66(24):16120-16127). Rosseneu et al. have also insisted that a hydrophobic angle of less than 180° is required for LCAT activation (WO 93/25581).

However, despite the progress in elucidating “rules” for designing ApoA-I agonists, to date the best ApoA-I agonists are reported as having less than 40% of the activity of intact ApoA-I. None of the peptide agonists described in the literature have been demonstrated to be useful as a drug. Thus, there is a need for the development of a stable molecule that mimics the activity of ApoA-I and which is relatively simple and cost-effective to produce. Preferably, candidate molecules would mediate both indirect and direct RCT. Such molecules would be smaller than existing peptide agonists, and have broader functional spectra. However, the “rules” for designing efficacious mediators of RCT have not been fully elucidated and the principles for designing organic molecules with the function of ApoA-I are unknown.

### SUMMARY OF THE INVENTION

In accordance with one preferred embodiment of the present invention, a mediator of reverse cholesterol transport comprising a molecule comprising an acidic region, a lipophilic or aromatic region and a basic region (the “Molecular Model”) is disclosed. The Molecular Model in its simplest form could be a molecule containing an acidic region and a basic region with a lipophilic backbone or scaffold. The molecule has a structure adapted to complex with HDL and/or LDL cholesterol and thereby enhance reverse cholesterol transport.

The mediator of reverse cholesterol transport preferably has between 3 and 10 amino acid residues or analogs or any non-peptide compound containing a basic group and an acid group with a lipophiilic scaffold, thereof, and comprises the sequence: X1—X2—X3, wherein: X1 is an acidic amino acid; X2 is an aromatic or a lipophilic amino acid; X3 is a basic amino acid; and wherein the amino terminal further comprises a first protecting group, and the carboxy terminal further comprises a second protecting group. The first and second protecting groups are independently selected from the group consisting of an acetyl, phenylacetyl, pivolyl, 9-fluorenylmethyloxycarbonyl, 2-napthylic acid, nicotinic acid, a CH₃—CH₂)ₙ—CO— where n ranges from 3 to 20, and an amide of acetyl, phenylacetyl, di-tert-butyl-4-hydroxy-phenyl, naphthyl, substituted naphthyl, f-MOC, biphenyl, substituted phenyl, substituted heterocycles, alkyl, aryl, substituted aryl, cycloalkyl, fused cycloalkyl, saturated heteroaryl, substituted saturated heteroaryl and the like. The C-terminal is capped with an amine such as RNH₂ where R=di-tert-butyl-4-hydroxy-phenyl, naphthyl, substituted naphthyl, f-MOC, biphenyl, substituted phenyl, substituted heterocycles, alkyl, aryl, substituted aryl, cycloalkyl, fused cycloalkyl, saturated heteroaryl, substituted saturated heteroaryl and the like. The sequence: X1—X2—X3 could be scrambled in any of all possible ways to provide compounds that retain the basic features of the Molecular Model and could be comprised of 3 to 10 amino acid residues.

In one embodiment of the amino acid-derived mediator of reverse cholesterol transport, one or more of X1, X2 or X3 are D or other modified synthetic amino acid residues to provide metabolically stable molecules. This could also be achieved by peptidomimetic approach i.e. reversing the the peptide bonds in the backbone or similar groups. In some preferred embodiments, X2 is biphenylalanine. In particularly preferred embodiments, the mediators of reverse cholesterol transport of the present invention are any of SEQ ID NOS 1-176 or may be selected from the compounds shown in Table 5. In some preferred embodiments, the mediator of reverse cholesterol transport includes the sequence EFR or RFE.

A method for enhancing RCT in an animal is disclosed in accordance with another preferred aspect of the present invention. The method comprises administering to the animal an effective amount of an amino acid-derived composition, comprising the sequence: X1—X2—X3, wherein: X1 is an acidic amino acid; X2 is an aromatic or lipophilic amino acid; X3 is a basic amino acid; and wherein the amino terminal further comprises a first protecting group, and the carboxy terminal further comprises a second protecting group, wherein the first and second protecting groups are independently selected from the group consisting of an acetyl, phenylacetyl, pivolyl, 9-fluorenylmethyloxycarbonyl, 2-napthylic acid, nicotinic acid, a CH₃—CH₂)ₙ—CO— where n ranges from 3 to 20, and di-tert-butyl-4-hydroxy-phenyl, naphthyl, substituted naphthyl, f-MOC, biphenyl, substituted phenyl, substituted heterocycles, alkyl, aryl, substituted aryl, cycloalkyl, fused cycloalkyl, saturated heteroaryl, substituted saturated heteroaryl and the like. The C-terminal is capped with an amine such as RNH₂ where R=di-tert-butyl-4-hydroxy-phenyl, naphthyl, substituted naphthyl, f-MOC, biphenyl, substituted phenyl, substituted heterocycles, alkyl, aryl, substituted aryl, cycloalkyl, fused cycloalkyl, saturated heteroaryl, substituted saturated heteroaryl and the like. The sequence: X1—X2—X3 could be scrambled in any of all possible ways to provide compounds that retain the basic features of the Molecular Model and could be comprised of 3 to 10 amino acid residues.

In accordance with another aspect of the present invention, a substantially pure amino acid-derived substance is disclosed for treating and/or preventing hypercholesterolemia and/or atherosclerosis in a mammal. The substance has an amino and a carboxy terminal and comprises an L or D enantiomer of an acidic amino acid residue or modified synthetic amino acid or derivative thereof, an L or D enantiomer of a lipophilic amino acid residue or derivative or modified synthetic amino acid thereof, and an L or D enantiomer of a basic amino acid residue or derivative or modified synthetic amino acid thereof. The amino terminal further comprises a first protecting group, and the carboxy terminal further comprises a second protecting group, wherein said first and second protecting groups are independently selected from the group consisting of an acetyl, phenylacetyl, pivolyl, 9-fluorenylmethyloxycarbonyl, 2-napthylic acid, nicotinic acid, and, di-tert-butyl-4-hydroxy-phenyl, naphthyl, substituted naphthyl, f-MOC, biphenyl, substituted phenyl, substituted heterocycles, alkyl, aryl, substituted aryl, cycloalkyl, fused cycloalkyl, saturated heteroaryl, substituted saturated heteroaryl and the like. The C-terminal is capped with an amine such as RNH₂ where R=di-tert-butyl-4-hydroxy-phenyl, naphthyl, substituted naphthyl, f-MOC, biphenyl, substituted phenyl, substituted heterocycles, alkyl, aryl, substituted aryl, cycloalkyl, fused cycloalkyl, saturated heteroaryl, substituted saturated heteroaryl and the like. The sequence: X1—X2—X3 could be scrambled in any of all possible ways to provide compounds that retain the basic features of the Molecular Model and could be comprised of 3 to 10 amino acid residues.

The substance has at least one of the following properties: (1) it binds to LDL and HDL mimicking ApoA-I binding to LDL and HDL, (2) it binds preferentially to liver, (3) it enhances LDL uptake by liver LDL-receptors, (4) it lower the levels of LDL, IDL, and VLDL cholesterol, (5) it increases the levels of HDL cholesterol, and (6) it improves plasma lipoprotein profiles.

In accordance with another aspect of the present invention, a composition suitable for oral administration is disclosed for ameliorating or preventing a symptom of hypercholesterolemia. The composition comprises an amino acid-derived molecule having an acidic region, a lipophilic region and a basic region. The amino acid-derived molecule also has a first protecting group attached to an amino terminal and a second protecting group attached to a carboxyl terminal. The amino acid-derived molecule may optionally comprise at least one D amino acid residue.

In accordance with another mode of the present invention, a peptide mediator of RCT is disclosed. The mediator comprises the sequence: Xa-Xb-X1—X2—X3-Xc-Xd, wherein Xa is an acylated amino acid residue; Xb is any 0-10 amino acid residues; X1—X2—X3 are selected independently from an acidic amino acid residue or derivative thereof, a lipophilic amino acid residue or derivative thereof, and a basic amino acid residue or derivative thereof; Xc is any 0-10 amino acid residues; and Xd is an amidated amino acid residue. The peptide mediator preferably has 15 or fewer amino acid residues and optionally may comprise of at least one D amino acid residue or modified synthetic amino acid.

In accordance with another preferred embodiment of the present invention, administration of a composition suitable for oral administration which includes an amino acid-derived molecule having an acidic region, a lipophilic region and a basic region for treatment and/or prevention of hypercholesterolemia or atherosclerosis is disclosed. The amino acid-derived molecule also has a first protecting group attached to an amino terminal and a second protecting group attached to a carboxyl terminal. The amino acid-derived molecule may optionally comprise at least one D amino acid residue.

In accordance with another embodiment of the present invention, an RCT mediator is disclosed, comprising a compound selected from the group consisting of the synthetic compounds 1-96 of Table 5.

In accordance with another embodiment of the present invention, an RCT mediator is disclosed, comprising a compound selected from the group consisting of SEQ ID NOS: 1 and 107-117.

In accordance with another embodiment of the present invention, an RCT mediator is disclosed, comprising a compound selected from the group consisting of SEQ ID NOS: 1, 26-36, 42, 45-47, 56-58, 68-70, 72-74, 76, 80, 81, 83-90 and 92-95.

In accordance with another embodiment of the present invention, an RCT mediator is disclosed, comprising a compound selected from the group consisting of SEQ ID NO: 1.

In accordance with another embodiment of the present invention, an RCT mediator is disclosed, comprising a compound selected from the group consisting of SEQ ID NO: 113.

In accordance with another embodiment of the present invention, an RCT mediator is disclosed, comprising a compound selected from the group consisting of SEQ ID NO: 34.

In accordance with another embodiment of the present invention, an RCT mediator is disclosed, comprising a compound selected from the group consisting of SEQ ID NO: 86.

In accordance with another embodiment of the present invention, an RCT mediator is disclosed, comprising a compound selected from the group consisting of SEQ ID NO: 91.

In accordance with another embodiment of the present invention, an RCT mediator is disclosed, comprising a compound selected from the group consisting of SEQ ID NO: 96.

In accordance with another embodiment of the present invention, an RCT mediator is disclosed, comprising a compound selected from the group consisting of SEQ ID NO: 145.

In accordance with another embodiment of the present invention, an RCT mediator is disclosed, comprising a compound selected from the group consisting of SEQ ID NO: 146.

In accordance with another embodiment of the present invention, an RCT mediator is disclosed, comprising a compound selected from the group consisting of SEQ ID NO: 118.

In accordance with another preferred embodiment of the present invention a method is disclosed for treating or preventing hypercholesterolemia and/or atherosclerosis. The method comprises administering to a mammal in need thereof an amount of a composition selected from the group consisting of SEQ ID NOS: 1-176 (Table 3) and synthetic compounds 1-96 (Table 5), wherein the amount is sufficient to enhance RCT and/or cause regression of existing atherosclerotic lesions or reduce formation of the lesions. More preferably, the composition used for treating or preventing hypercholesterolemia and/or atherosclerosis is selected from the group consisting of SEQ ID NOS: 1, 113, 34, 86, 91, 96, 145, 146, and 118, wherein the amount is sufficient to enhance RCT and/or cause regression of existing atherosclerotic lesions or reduce formation of the lesions. In one variation to the method, the step of administering is accomplished via an oral route. In another variation to the method, the step of administering is combined with administration of a bile acid-binding resin, niacin, a statin, or a combination thereof.

In accordance with another preferred embodiment of the present invention, an in vitro screening method is disclosed for identifying test compounds that are likely to enhance reverse cholesterol transport in vivo. The method comprises: measuring cholesterol accumulation in liver cells in vitro in the presence and absence of the test compounds; measuring cholesterol accumulation and/or efflux in AcLDL-loaded macrophages in vitro in the presence and absence of test compounds; and identifying test compounds that enhance cholesterol accumulation in liver cells and reduce cholesterol levels in macrophages.

In a variation to the screening method, cholesterol levels are also measured in OxLDL-loaded vascular smooth muscle cells in vitro in the presence and absence of test compounds. Accordingly, the step of identifying test compounds further comprises identifying compounds that enhance cholesterol accumulation in liver cells and reduce cholesterol levels in macrophages and/or reduce cholesterol levels in vascular smooth muscle cells.

In one embodiment of the screening method, the liver cells are human HepG2 hepatoma cells. In another embodiment, the macrophages are human THP-1 cells. In another embodiment, the vascular smooth muscle cells are primary aortic smooth muscle cells.

In another variation, the in vitro screening method comprises the steps of: measuring cholesterol accumulation in liver cells in vitro in the presence and absence of the test compounds; measuring cholesterol levels in AcLDL-loaded vascular smooth muscle cells in vitro in the presence and absence of test compounds; and identifying test compounds that enhance cholesterol accumulation in liver cells and reduce cholesterol levels in vascular smooth muscle cells.

### BRIEF DESCRIPTION OF THE DRAWINGS

FIG. 1 shows a schematic representation of solid phase peptide synthesis.

FIG. 2 illustrates association of the amino acid-derived compositions of the present invention with lipoproteins and albumin. Radiolabeled compounds were incubated with LDLR−/− mouse plasma at RT for 2 hrs. Following incubation, the mixture was subjected to agarose gel electrophoresis. Radioactivity was quantified in the bands representing LDL, HDL, and Albumin. Lipoproteins and Albumin bound radioactivity is expressed as percentage of applied radioactivity.

FIG. 3 shows that the amino acid-derived compositions of the present invention bind to the liver in ApoA1−/− male mice. ApoAl−/− male mice were injected with 12 ug/mouse of radiolabeled compounds. 36 min livers were harvested, radioactivity quantified and adjusted per g of wet tissue. Liver bound radioactivity is expressed as % of total cpm. Each bar represents the mean±SEM of 4 mice.

FIG. 4 shows the organ distribution of SEQ ID NO 1 and preferential uptake by the liver. ApoA1−/− male mice were injected with 12 ug of radiolabeled SEQ ID NO: 1. 36 min later organs were harvested, radioactivity quantified and adjusted per g of wet tissue. Each bar represents the mean±SEM of 4 mice.

FIG. 5 shows that complexing of Human 125I-LDL with SEQ ID NO: 1 improved its binding to the liver. LDL delivery to the liver is enhanced by SEQ ID NO 1. ¹²⁵I-LDL alone or ¹²⁵I-LDL complexed with SEQ ID NO: 1 were injected into male mice of the deficient genotypes as indicated. 36 min later livers were collected at and radioactivity quantified. Liver bound radioactivity is expressed as % of injected radioactivity. Each bar represents the mean±SEM of 4 mice.

FIG. 6 shows that SEQ ID NO 1-LDL complexes bind to the LDL receptor on liver. Binding of ¹²⁵I-LDL alone or ¹²⁵I-LDL complexed with SEQ ID NO: 1 to the liver of LDLR−/− mice were subtracted from their respective binding to the liver of A-I−/− mice. Result of subtraction indicates on dramatic increase in binding of the complex to the LDL-receptors. Each bar represents the mean±SEM of 4 mice.

FIG. 7 shows the effects of SEQ ID NO 1 on clearance of human LDL from the blood of ApoA-I-deficient mice. ¹²⁵I-LDL alone or ¹²⁵I-LDL complexed with SEQ ID NO: 1 were injected into ApoA1−/− mice. At the indicated time points plasma was obtained, and 10%TCA precipitable radioactivity was measured. 100 % is equal to blood radioactivity determined 10 min after injection. Each value represents the mean±1 SEM of 4 animals.

FIG. 8 shows the effect of SEQ ID NO 1 on LDL organ distribution in ApoA-I-deficient and LDL receptor-deficient mice. Radioactivity,%=(organ bound radioactivity/blood radioactivity)×100%. Approximately 90% of total detected radioactivity (at the 36 min time point) is the blood radioactivity.

FIG. 9 shows the effect of SEQ ID NO 1 on plasma VLDL cholesterol levels. Mice were divided into two groups (4 mice in each group). SEQ ID NO: 1 or PBS were injected intravenously into experimental or control mice, respectively. At the indicated time points plasma was obtained, combined within each group, and applied on Superose 6 column. Each time point on the curve represents the Mean±SEM obtained from > or =3 chromatographic profiles.

FIG. 10 shows the effect of SEQ ID NO 1 on plasma IDL/LDL cholesterol levels. Mice were divided into two groups (4 mice in each group). SEQ ID NO: 1 or PBS were injected intravenously into experimental or control mice, respectively. At the indicated time points plasma was obtained, combined within each group, and applied on Superose 6 column. Each time point on the curve represents the Mean±SEM obtained from > or =3 chromatographic profiles.

FIG. 11 shows the effect of SEQ ID NO 1 on plasma HDL levels. Mice were divided into two groups (4 mice in each group). SEQ ID NO: 1 or PBS were injected intravenously into experimental or control mice, respectively. At the indicated time points plasma was obtained, combined within each group, and applied on Superose 6 column. Each time point on the curve represents the Mean±SEM obtained from > or =3 chromatographic profiles.

FIG. 12 shows linear regression analysis of the effects of SEQ ID NO 1 on plasma VLDL cholesterol levels.

FIG. 13 shows linear regression analysis of the effects of SEQ ID NO 1 on plasma IDL/LDL cholesterol levels.

FIG. 14 shows linear regression analysis of the effects of SEQ ID NO 1 on plasma HDL levels.

FIG. 15 shows the effect of time-released SEQ ID NO 1 on plasma lipoprotein profile. Pumps containing SEQ ID NO: 1 or PBS were surgically inserted in canulated Chow fed mice. Pumps flow rate was 8 ul/hr, which provided indicated in picture amount of SEQ ID NO: 1 per hr. Animals were switched on HFC diet immediately after surgery. 20 hr later plasma was obtained, combined within each group (4-6 mice), and subjected to FPLC and agarose gel electrophoresis to monitor cholesterol and phospholipids distribution among different lipoprotein classes (for clarity, the data for phospholipids is not shown). Effect is expressed as % of change compared to PBS control.

FIG. 16 shows the long-term (20 hours) effects of infusion of various amino acid-derived compositions of the present invention on plasma lipoprotein profiles. Pumps containing SEQ ID NO: 1 or PBS were surgically inserted in canulated Chow fed mice. Pumps flow rate was 8 ul/hr, which provided 30-40 ug of peptide per hr. Animals were switched on HFC diet immediately after surgery. 20 hr later plasma was obtained, combined within each group (4-6 mice), and subjected to FPLC and agarose gel electrophoresis to monitor cholesterol and phospholipids distribution among different lipoprotein classes. Effect is expressed as % of change compared to PBS control.

FIG. 17 shows the long-term (160 hours) effects of infusion of various amino acid-derived compositions of the present invention on plasma lipoprotein profiles. Pumps containing SEQ ID NO: 1 or PBS were surgically inserted in canulated Chow fed mice. Pumps flow rate was 1 ul/hr, which provided indicated in graph amount of peptide per hr. Animals were switched on HFC diet immediately after surgery. 160 hr later plasma was obtained, combined within each group (4-6 mice), and subjected to FPLC and agarose gel electrophoresis to monitor cholesterol and phospholipids distribution among different lipoprotein classes (for clarity, the data for phospholipids is not shown). Effect is expressed as % of change compared to PBS control.

FIG. 18 shows the effects of various peptides (SEQ ID Nos: 34, 86, 91, 96, 35 and 36) of the present invention on PLTP enzyme activity. SEQ ID Nos: 34, 86, 91 and 96 resulted in activation of PLTP.

FIG. 19 shows the acute effects of oral administration of SEQ ID NO: 91 on plasma lipoprotein profiles and excretion of cholesterol in bile acid.

FIG. 20 shows effect of ad lib (via the drinking water) administration of AVP-26249 (SEQ ID NO: 91) on plasma lipoprotein profiles of Chow fed ApoE−/− mice.

FIG. 21 shows effects of ad lib (via the drinking water) administration of AVP-26249 (SEQ ID NO: 91), AVP-26451 (SEQ ID NO: 145), AVP-26452 (SEQ ID NO: 146) and AVP-26355 (SEQ ID NO: 118) on plasma lipoprotein profiles of ApoE−/− mice fed high fat diets.

FIG. 22 shows effects of ad lib (via the drinking water) administration of AVP-26249 (SEQ ID NO: 91), AVP-26451 (SEQ ID NO: 145), AVP-26452 (SEQ ID NO: 146) and AVP-26355 (SEQ ID NO: 118) on amount of cholesterol excreted by ApoE−/− mice fed high fat diets.

FIG. 23 is a schematic diagram showing in vitro cell culture triangle screening method for test compounds likely to enhance RCT in vivo.

FIG. 24 shows effect of AVP-26249 (SEQ ID NO: 91) and AVP-26452 (SEQ ID NO: 146) on LDL-mediated accumulation of cholesterol in HepG2 cells.

FIG. 25 shows effect of AVP-26249 (SEQ ID NO: 91) on Ac-LDL-mediated accumulation of cholesterol (TC) and cholesteryl ester (CE) in human macrophages.

FIG. 26 shows effect of AVP-26249 (SEQ ID NO: 91) and AVP-26452 (SEQ ID NO: 146) on Oxidized-LDL (Ox-LDL) mediated accumulation of cholesterol (TC) and cholesteryl ester (CE) in human vascular smooth muscle cells.

FIG. 27 shows effect of AVP-26249 (SEQ ID NO: 91) and AVP-26452 (SEQ ID NO: 146) on cholesterol efflux from Ac-LDL preloaded human macrophages.

FIG. 28 shows effect of AVP-26249 (SEQ ID NO: 91) on development of atherosclerotic lesions in aorta of ApoE−/− mice. ApoE−/− male mice were maintained on Chow diet for 4 weeks and on HFD (1.25% of cholesterol) for 9.3 weeks. Mice received AVP-26249 “ad lib” via the drinking water at concentrations of 0, 1.4 and 2.8 mpk for 13.3 weeks. At the end of experiment aortas were isolated and assessed for progression of atherosclerotic lesions.

FIG. 29 shows effect of AVP-26452 (SEQ ID NO: 146) on development of atherosclerotic lesions in aorta of ApoE−/− mice. ApoE−/− male mice were maintained on Chow diet for 4 weeks and on HFD (1.25% of cholesterol) for 9.3 weeks. Mice received AVP-26452 “ad lib” via the drinking water at concentrations of 0, 1.4 and 2.8 mpk for 13.3 weeks. At the end of experiment aortas were isolated and assessed for progression of atherosclerotic lesions.

FIG. 30 is a schematic diagram showing pathways in cholesterol transport and metabolism. Abbreviations include CE, cholesterol ester; PLTP, Phospholipid transfer protein; TG, triglyceride; LDL, low density lipoprotein; HDL, high density lipoprotein; IDL, intermediate density lipoprotein; LCAT, Lecithin:cholesterol acyltransferase.

### DETAILED DESCRIPTION OF THE PREFERRED EMBODIMENT

The mediators of RCT in preferred embodiments of the invention mimic ApoA-I function and activity. In a broad aspect, these mediators are molecules comprising three regions, an acidic region, a lipophilic (e.g., aromatic) region, and a basic region. The molecules preferably contain a positively charged region, a negatively charged region, and an uncharged, lipophilic region. The locations of the regions with respect to one another can vary between molecules; thus, in a preferred embodiment, the molecules mediate RCT regardless of the relative positions of the three regions within each molecule. Whereas in some preferred embodiments, the molecular template or model comprises an acidic amino acid-derived residue, a lipophilic amino acid-derived residue, and a basic amino acid-derived residue, linked in any order to form a mediator of RCT, in other preferred embodiments, the molecular model can be embodied by a single residue having acidic, lipophilic and basic regions, such as for example, the amino acid, phenylalanine (SEQ ID NO 127).

In some preferred embodiments, the molecular mediators of RCT comprise trimers of natural D- or L- amino acids, amino acid analogs (synthetic or semisynthetic), and amino acid derivatives. For example, a trimer may include an acidic amino acid residue or analog thereof, an aromatic or lipophilic amino acid residue or analog thereof, and a basic amino acid residue or analog thereof, the residues being joined by peptide or amide bond linkages. For example, the trimer sequence EFR comprises an acidic residue (glutamic acid), an aromatic residue (phenylalanine) and a basic amino acid residue (arginine). In other preferred aspects of the invention, the molecular mediators may be larger amino acid-based compounds which comprise one or more of the amino acid trimers. For example the decapeptide, YEFRDRMRTH, comprises the acidic-aromatic-basic trimer sequence, EFR, discussed above or efr or rfe, i.e cotaining d-amino acid residues or E-(4-Phenyl)-FR or modified or synthetic or semisynthetic amino acid residues.

While the molecular mediators of RCT share the common aspect of reducing serum cholesterol through enhancing direct and/or indirect RCT pathways (i.e., increasing cholesterol efflux), the preferred mediators may exhibit inter alia one or more of the following specific functional attributes: ability to form amphipathic helical structures or sub-structures thereof in the presence or absence of lipid, ability to bind lipids, ability to form pre-β-like or HDL-like complexes, ability to activate LCAT, and ability to increase serum HDL concentration.

To date, efforts at designing ApoA-I agonists have focused on the 22-mer unit structures, e.g., the “consensus 22-mer” of Anantharamaiah et al., 1990, Arteriosclerosis 10(1):95-105; Venkatachalapathi et al., 1991, Mol. Conformation and Biol. Interactions, Indian Acad. Sci. B:585-596, which are capable of forming amphipathic α-helices in the presence of lipids. (See e.g., U.S. Pat. No. 6,376,464 directed at peptide mimetics derived from modifications of the consensus 22-mer). In accordance with a preferred aspect of the present invention, relatively short (less than about 10 amino acid residues) amphipathic α-helical sub-structures which are derived from any of multiple α-helical domains of the intact ApoA-I were synthesized and tested as mediators of RCT. There are several advantages of using such relatively short peptides compared to longer 22-mers. For example, the shorter mediators of RCT are easier and less costly to produce, they are chemically and conformationally more stable, the preferred conformations remain relatively rigid, there is little or no intra-molecular interactions within the peptide chain, and the shorter peptides exhibit a higher degree of oral availability. Multiple copies of these shorter peptides might bind to the HDL or LDL producing the same effect of a more restrained large peptide. Although ApoA-I multifunctionality may be based on the contributions of its multiple α-helical domains, it is also possible that even a single function of ApoA-I, e.g., LCAT activation, can be mediated in a redundant manner by more than one of the a-helical domains. Thus, in a preferred aspect of the present invention, multiple functions of ApoA-I may be mimicked by the disclosed mediators of RCT which are directed to a single sub-domain.

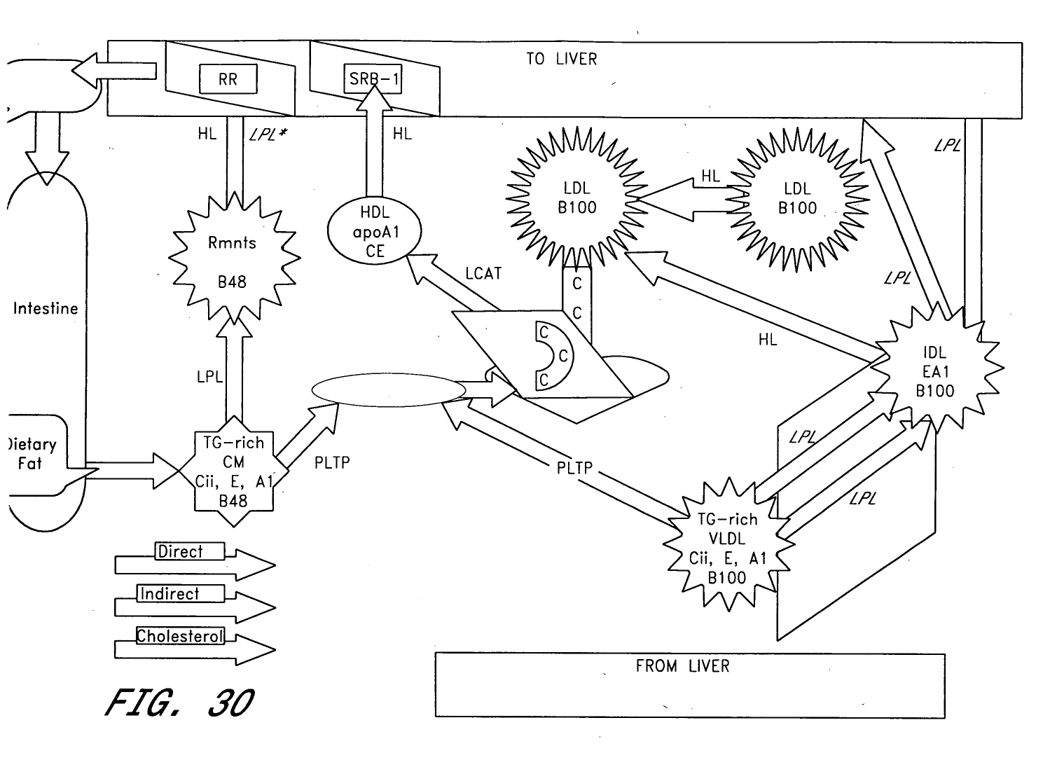

Three functional features of ApoA-I are widely accepted as major criteria for ApoA-I agonist design: (1) ability to associate with phospholipids; (2) ability to activate LCAT; and (3) ability to promote efflux of cholesterol from the cells. Some of the cholesterol transport and metabolism pathways are illustrated in FIG. 30. The molecular mediators of RCT in accordance with some modes of the present invention may exhibit only the last functional feature-ability to increase RCT. However, quite a few other properties of ApoA-I, which are often overlooked, make ApoA-I a particularly attractive target for therapeutic intervention. For example, ApoA-I directs the cholesterol flux into the liver via a receptor-mediated process and modulates pre-β-HDL (primary acceptor of cholesterol from peripheral tissues) production via a PLTP driven reaction. However, these features allow broadening of the potential usefulness of ApoA-I mimetic molecules. This, entirely novel approach to viewing ApoA-I mimetic function, will allow use of the peptides or amino acid-derived small molecules, which are disclosed herein, to facilitate direct RCT (via HDL pathway) as well as indirect RCT (i.e., to intercept and clear the LDLs from circulation, by redirecting their flux to the liver); see e.g., FIG. 30. To be capable of enhancing indirect RCT, the molecular mediators of the present invention will preferably be able to associate with phospholipids and bind to the liver (i.e., to serve as ligand for liver lipoprotein binding sites).

Thus, a goal of the research efforts which led to the present invention was to identify, design, and synthesize the short (less than about 10 amino acid residues), stable peptide mediators of RCT that exhibit preferential lipid binding conformation, increase cholesterol flux to the liver by facilitating direct and/or indirect reverse cholesterol transport, improve the plasma lipoprotein profile, and subsequently prevent the progression or/and even promote the regression of atherosclerotic lesions.

Our peptide design strategy was: (1) to determine relatively short (3-15 amino acid residues) interactive regions within the amphipathic a-helical domains of ApoA-I; (2) to base our first generation of peptides on exact ApoA-I sequence; (3) to design peptides according to the general rules emerged from investigation of amphipathic α-helical domains of ApoA-I; (4) to define the lower limits of peptide sequence length, the critical amino acid residues, and the exact topography in the shortest possible peptide, which still exhibits both amphipathic α-helical secondary structure and ApoA-I activity in vitro and in vivo; and (5) to incorporate defined physical chemical properties into design of even shorter small-molecule-like peptides and/or other small molecules that evolved into the Molecular Model as described above.

The mediators of RCT of the invention can be prepared in stable bulk or unit dosage forms, e.g., lyophilized products, that can be reconstituted before use in vivo or reformulated. The invention includes the pharmaceutical formulations and the use of such preparations in the treatment of hyperlipidemia, hypercholesterolemia, coronary heart disease, atherosclerosis, and other conditions such as endotoxemia causing septic shock.

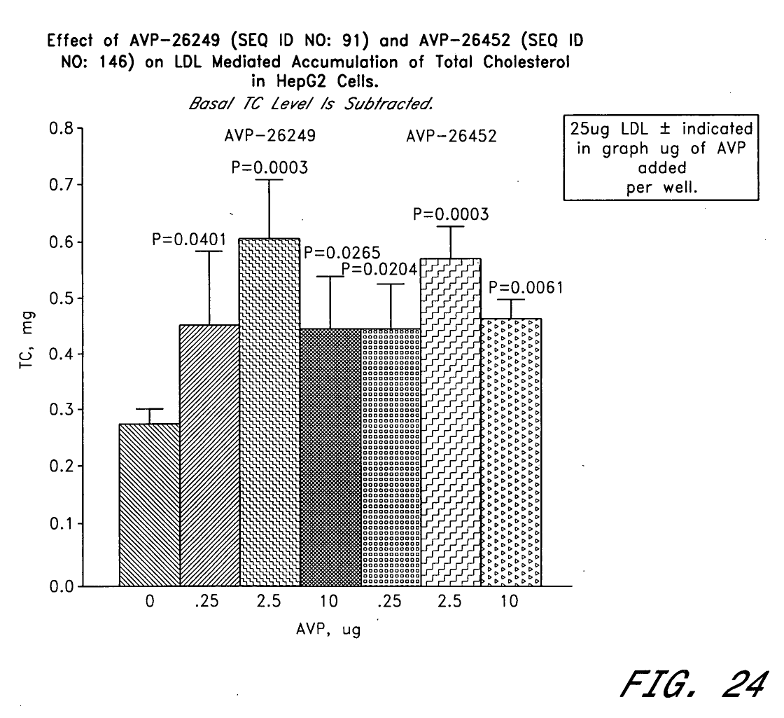

The invention is illustrated by working examples which demonstrate that the mediators of RCT of the invention associate with the HDL and LDL component of plasma, and can increase the concentration of HDL and pre-β-HDLparticles, and lower plasma levels of LDL. Thus promote direct and indirect RCT. The mediators of RCT of the invention increase human LDL mediated cholesterol accumulation in human hepatocytes (HepG2 cells) (as shown in FIG. 24). The mediators of RCT are also efficient at activating PLTP and thus promote the formation of pre-p-HDL particles. Increase of HDL cholesterol served as indirect evidence of LCAT involvement (LCAT activation was not shown directly (in vitro)) in the RCT. Use of the mediators of RCT of the invention in vivo in animal models results in an increase in serum HDL concentration.

The invention is set forth in more detail in the subsections below, which describe the composition and structure of the mediators of RCT; structural and functional characterization; methods of preparation of bulk and unit dosage formulations; and methods of use.

Peptide Structure and Function

The mediators of RCT of the invention are generally peptides, or analogues thereof, which mimic the activity of ApoA-I. The mediators of RCT are composed of less than about 10 amino acid residues, or an analogue thereof. In some embodiments, at least one amide linkage in the peptide is replaced with a substituted amide, an isostere of an amide or an amide mimetic. Additionally, one or more amide linkages can be replaced with peptidomimetic or amide mimetic moieties which do not significantly interfere with the structure or activity of the peptides. Suitable amide mimetic moieties are described, for example, in Olson et al., 1993, J. Med. Chem. 36:3039-3049.

A preferred feature of the peptides is their ability to form amphipathic α-helices or substructures. By amphipathic is meant that the α-helix has opposing hydrophilic and hydrophobic faces oriented along its long axis, i.e., one face of the helix projects mainly hydrophilic side chains while the opposite face projects mainly hydrophobic side chains.

As will be discussed more thoroughly below in conjunction with altered or mutated forms of the peptides, certain amino acid residues can be replaced with other amino acid residues such that the hydrophilic and hydrophobic faces of the helix formed by the peptides may not be composed entirely of hydrophilic and hydrophobic amino acids, respectively. Thus, it is to be understood that when referring to the amphipathic α-helix formed by the peptides of the invention, the phrase “hydrophilic face” refers to a face of the helix having overall net hydrophilic character. The phrase “hydrophobic face” refers to a face of the peptide having overall net hydrophobic character. While not intending to be bound by any particular theory, it is believed that certain structural and/or physical properties of the amphipathic helical structures formed by the peptides may contribute to their activity. These properties include the degree of amphipathicity, overall hydrophobicity, mean hydrophobicity, hydrophobic and hydrophilic angles, hydrophobic moment, mean hydrophobic moment, and net charge of the α-helix. The degree of amphipathicity (degree of asymmetry of hydrophobicity) can be conveniently quantified by calculating the hydrophobic moment (μ_{H}) of the helix. Methods for calculating μ_{H} for a particular peptide sequence are well-known in the art, and are described, for example in Eisenberg, 1984, Ann. Rev. Biochem. 53:595-623. The actual μ_{H} obtained for a particular peptide will depend on the total number of amino acid residues composing the peptide. Thus, it is generally not informative to directly compare μ_{H} for peptides of different lengths.

The amphipathicities of peptides of different lengths can be directly compared by way of the mean hydrophobic moment (<μ_{H}>). The mean hydrophobic moment can be obtained by dividing μ_{H} by the number of residues in the helix (i.e., <μ_{H}>=μ_{H}/N). Generally, the preferred peptides, which exhibit a <μ_{H}> in the range of 0.45 to 0.65, as determined using the normalized consensus hydrophobicity scale of Eisenberg (Eisenberg, 1984, J Mol. Biol. 179:125-142), are considered to be within the scope of the present invention, with a <μ_{H}>in the range of 0.50 to 0.60 being preferred.

The overall or total hydrophobicity (Hₒ) of a peptide can be conveniently calculated by taking the algebraic sum of the hydrophobicities of each amino acid residue in the peptide:

$$( i . e . , {H}_{o} = {\sum }_{iα 1}^{N} {H}_{i} ) ,$$

where N is the number of amino acid residues in the peptide and Hᵢ is the hydrophobicity of the ith amino acid residue). The mean hydrophobicity (<Hₒ>) is the hydrophobicity divided by the number of amino acid residues (i.e., <Hₒ>=Hₒ/N). Generally, peptides that exhibit a mean hydrophobicity in the range of −0.050 to −0.070, as determined using the normalized consensus hydrophobicity scale of Eisenberg (Eisenberg, 1984, J. Mol. Biol. 179:125-142) are considered to be within the scope of the present invention, with a mean hydrophobicity in the range of −0.030 to −0.055 being preferred.

The total hydrophobicity of the hydrophobic face (Hₒ^{pho}) of an amphipathic helix can be obtained by taking the sum of the hydrophobicities of the hydrophobic amino acid residues which fall into the hydrophobic angle as defined below:

$${H}_{o}^{pho} = {\sum }_{iα 1}^{N} {{H}_{i}}^{*} ,$$

where Hᵢ is as previously defined and N_{H} is the total number of hydrophobic amino acids in the hydrophobic face). The mean hydrophobicity of the hydrophobic face (<Hₒ^{pho}>) is Hₒ^{pho}/N_{H} where N_{H} is as defined above. Generally, peptides which exhibit a <Hₒ^{pho}> in the range of 0.90 to 1.20, as determined using the consensus hydrophobicity scale of Eisenberg (Eisenberg, 1984, supra; Eisenberg et al., 1982, supra) are considered to be within the scope of the present invention, with a <Hₒ^{pho}> in the range of 0.94 to 1.10 being preferred.

The hydrophobic angle (pho angle) is generally defined as the angle or arc covered by the longest continuous stretch of hydrophobic amino acid residues when the peptide is arranged in the Schiffer-Edmundson helical wheel representation (i.e., the number of contiguous hydrophobic residues on the wheel multiplied by 20°). The hydrophilic angle (phi angle) is the difference between 360° and the pho angle (i.e., 360°-pho angle). Those of skill in the art will recognize that the pho and phi angles will depend in part, on the number of amino acid residues in the peptide.

Amphipathic peptides and molecular mediators, having acidic, aromatic and basic regions, in accordance with preferred aspects of the present invention, are expected to bind phospholipids by pointing their hydrophobic faces towards the alkyl chains of the lipid moieties. It is believed that the hydrophobic cluster will generate sufficiently strong lipid binding affinities for the peptides of the invention. Since lipid binding is a prerequisite for LCAT activation, it is also believed that the hydrophobic cluster may enhance LCAT activation. In addition, aromatic residues are often found to be important in anchoring peptides and proteins to lipids (De Kruijff, 1990, Biosci. Rep. 10:127-130; O'Neil and De Grado, 1990, Science 250:645-651; Blondelle et al., 1993, Biochim. Biophys. Acta 1202:331-336).

Interactions between the peptides of the invention and lipids lead in preferred embodiments to the formation of peptide-lipid complexes. The type of complex obtained (comicelles, discs, vesicles or multilayers) will depend on the lipid:peptide molar ratio, with comicelles generally being formed at low lipid:peptide molar ratios and discoidal and vesicular or multilayer complexes being formed with increasing lipid:peptide molar ratios. This characteristic has been described for amphipathic peptides (Epand, The Amphipathic Helix, 1993) and for ApoA-I (Jones, 1992, Structure and Function of Apolipoproteins, Chapter 8, pp. 217-250). The lipid:peptide molar ratio also determines the size and composition of the complexes.

In the generally accepted structural model of ApoA-I, the amphipathic α-helices are packed around the edge of the discoidal HDL. In this model, the helices are assumed to be aligned with their hydrophobic faces pointing towards the lipid acyl chains (Brasseur et al., 1990, Biochim. Biophys. Acta 1043:245-252). The helices are arranged in an antiparallel fashion, and a cooperative effect between the helices is thought to contribute to the stability of the discoidal HDL complex (Brasseur et al., supra). Although it has been proposed that one factor which contributes to the stability of the HDL discoidal complex is the existence of ionic interactions between acidic and basic residues in ApoA-I resulting in the formation of intermolecular salt bridges or hydrogen bonds between residues on adjacent anti-parallel helices, such intermolecular interactions are not necessary to the activity of the molecular mediators. Thus, additional features of some of the mediators of RCT are their ability to form intermolecular hydrogen-bonds with one another when aligned in an antiparallel fashion with their hydrophobic faces pointing in the same direction, such as would be the case when the mediators are bound to lipids.

It is widely held that intramolecular hydrogen bond or salt bridge formation between acidic and basic residues, respectively, at positions i and i+3 of the helix stabilize the helical structure (Marqusee et al., 1985, PNAS. USA 84(24):8898-8902). However, such intramolecular interactions are minimal in the relatively small molecular mediators of the present invention.

As used herein, the abbreviations for the genetically encoded L-enantiomeric amino acids are conventional and are as follows: The D-amino acids are designated by lower case, e.g. D-alanine=a, etc.

TABLE 1

| Amino Acids | One-Letter Symbol | Common Abbreviation |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Certain amino acid residues in the peptide mediators of RCT can be replaced with other amino acid residues without significantly deleteriously affecting, and in many cases even enhancing, the activity of the peptides. Thus, also contemplated by the present invention are altered or mutated forms of the peptide mediators of RCT wherein at least one defined amino acid residue in the structure is substituted with another amino acid residue or derivative and/or analog thereof. As one of the features affecting the activity of the peptides of the invention may be their ability to form a-helices in the presence of lipids that exhibit the amphipathic and other properties described above, it will be recognized that in preferred embodiments of the invention, the amino acid substitutions are conservative, i.e., the replacing amino acid residue has physical and chemical properties that are similar to the amino acid residue being replaced.

For purposes of determining conservative amino acid substitutions, the amino acids can be conveniently classified into two main categories—hydrophilic and hydrophobic—depending primarily on the physical-chemical characteristics of the amino acid side chain. These two main categories can be further classified into subcategories that more distinctly define the characteristics of the amino acid side chains. For example, the class of hydrophilic amino acids can be further subdivided into acidic, basic and polar amino acids. The class of hydrophobic amino acids can be further subdivided into nonpolar and aromatic amino acids. The definitions of the various categories of amino acids that define ApoA-I are as follows:

The term “hydrophilic amino acid” refers to an amino acid exhibiting a hydrophobicity of less than zero according to the normalized consensus hydrophobicity scale of Eisenberg et al., 1984, J. Mol. Biol. 179:125-142. Genetically encoded hydrophilic amino acids include Thr (T), Ser (S), His (H), Glu (E), Asn (N), Gin (Q), Asp (D), Lys (K) and Arg (R).

The term “hydrophobic amino acid” refers to an amino acid exhibiting a hydrophobicity of greater than zero according to the normalized consensus hydrophobicity scale of Eisenberg, 1984, J. Mol. Biol. 179:1.25-142. Genetically encoded hydrophobic amino acids include Pro (P), Ile (I), Phe (F), Val (V), Leu (L), Trp (W), Met (M), Ala (A), Gly (G) and Tyr (Y).

The term “acidic amino acid” refers to a hydrophilic amino acid having a side chain pK value of less than 7. Acidic amino acids typically have negatively charged side chains at physiological pH due to loss of a hydrogen ion. Genetically encoded acidic amino acids include Glu (E) and Asp (D).

The term “basic amino acid” refers to a hydrophilic amino acid having a side chain pK value of greater than 7. Basic amino acids typically have positively charged side chains at physiological pH due to association with hydronium ion. Genetically encoded basic amino acids include His (H), Arg (R) and Lys (K).

The term “polar amino acid” refers to a hydrophilic amino acid having a side chain that is uncharged at physiological pH, but which has at least one bond in which the pair of electrons shared in common by two atoms is held more closely by one of the atoms. Genetically encoded polar amino acids include Asn (N), Gin (Q) Ser (S) and Thr (T).

The term “nonpolar amino acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded nonpolar amino acids include Leu (L), Val (V), Ile (I), Met (M), Gly (G) and Ala (A).

The term “aromatic amino acid” refers to a hydrophobic amino acid with a side chain having at least one aromatic or heteroaromatic ring. The aromatic or heteroaromatic ring may contain one or more substituents such as —OH, —SH, —CN, —F, —Cl, —Br, —I, —NO₂, —NO, —NH₂, —NHR, —NRR, —C(O)R, —C(O)OH, —C(O)OR, —C(O)NH₂, —C(O)NHR, —C(O)NRR and the like where each R is independently (C₁-C₆) alkyl, substituted (C₁-C₆) alkyl, (C₁-C₆) alkenyl, substituted (C₁-C₆) alkenyl, (C₁-C₆) alkynyl, substituted (C₁-C₆) alkynyl, (C₅-C₂₀) aryl, substituted (C₅-C₂₀) aryl, (C₆- C₂₆) alkaryl, substituted (C₆-C₂₆) alkaryl, 5-20 membered heteroaryl, substituted 5-20 membered heteroaryl, 6-26 membered alkheteroaryl or substituted 6-26 membered alkheteroaryl. Genetically encoded aromatic amino acids include Phe (F), Tyr (Y) and Trp (W).

The term “aliphatic amino acid” refers to a hydrophobic amino acid having an aliphatic hydrocarbon side chain. Genetically encoded aliphatic amino acids include Ala (A), Val (V), Leu (L) and Ile (I).

The amino acid residue Cys (C) is unusual in that it can form disulfide bridges with other Cys (C) residues or other sulfanyl-containing amino acids. The ability of Cys (C) residues (and other amino acids with —SH containing side chains) to exist in a peptide in either the reduced free —SH or oxidized disulfide-bridged form affects whether Cys (C) residues contribute net hydrophobic or hydrophilic character to a peptide. While Cys (C) exhibits a hydrophobicity of 0.29 according to the normalized consensus scale of Eisenberg (Eisenberg, 1984, supra), it is to be understood that for purposes of the present invention Cys (C) is categorized as a polar hydrophilic amino acid, notwithstanding the general classifications defined above.

As will be appreciated by those of skill in the art, the above-defined categories are not mutually exclusive. Thus, amino acids having side chains exhibiting two or more physical-chemical properties can be included in multiple categories. For example, amino acid side chains having aromatic moieties that are further substituted with polar substituents, such as Tyr (Y), may exhibit both aromatic hydrophobic properties and polar or hydrophilic properties, and can therefore be included in both the aromatic and polar categories. The appropriate categorization of any amino acid will be apparent to those of skill in the art, especially in light of the detailed disclosure provided herein.

Certain amino acid residues, called “helix breaking” amino acids, have a propensity to disrupt the structure of α-helices when contained at internal positions within the helix. Amino acid residues exhibiting such helix-breaking properties are well-known in the art (see, e.g., Chou and Fasman, Ann. Rev. Biochem. 47:251-276) and include Pro (P), Gly (G) and potentially all D-amino acids (when contained in an L-peptide; conversely, L-amino acids disrupt helical structure when contained in a D-peptide). While these helix-breaking amino acid residues fall into the categories defined above, with the exception of Gly (G), these residues are generally not used to substitute amino acid residues at internal positions within the helix-they are generally used to substitute 1-3 amino acid residues at the N-terminus and/or C-terminus of the peptide.

While the above-defined categories have been exemplified in terms of the genetically encoded amino acids, the amino acid substitutions need not be, and in certain embodiments preferably are not, restricted to the genetically encoded amino acids. Indeed, many of the preferred peptide mediators of RCT contain genetically non-encoded amino acids. Thus, in addition to the naturally occurring genetically encoded amino acids, amino acid residues in the peptide mediators of RCT may be substituted with naturally occurring non-encoded amino acids and synthetic amino acids.

Certain commonly encountered amino acids which provide useful substitutions for the peptide mediators of RCT include, but are not limited to, β-alanine (β-Ala) and other omega-amino acids such as 3-aminopropionic acid, 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (Phg); cyclohexylalanine (Cha); norleucine (Nle); naphthylalanine (Nal); 4-phenylphenylalanine, 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,4-diaminobutyric acid (Dbu); 2,3-diaminobutyric acid (Dab); p-aminophenylalanine (Phe (pNH₂)); N-methyl valine (MeVal); homocysteine (hCys), homophenylalanine (hPhe) and homoserine (hSer); hydroxyproline (Hyp), homoproline (hPro), N-methylated amino acids and peptoids (N-substituted glycines).

Other amino acid residues not specifically mentioned herein can be readily categorized based on their observed physical and chemical properties in light of the definitions provided herein.

The classifications of the genetically encoded and common non-encoded amino acids according to the categories defined above are summarized in Table 2, below. It is to be understood that Table 2 is for illustrative purposes only and does not purport to be an exhaustive list of amino acid residues and derivatives that can be used to substitute the peptide mediators of RCT described herein.

TABLE 2
CLASSIFICATIONS OF COMMONLY
ENCOUNTERED AMINO ACIDS

| Classification | Genetically Encoded | Non-Genetically Encoded |
|---|---|---|
| Hydrophobic | | |
| Aromatic | F, Y, W | Phg, Nal, Thi, Tic, Phe (4-Cl), |
| | | Phe (2-F), Phe (3-F), Phe (4-F), |
| | | hPhe |
| Nonpolar | L, V, I, M, G, A, P | t-BuA, t-BuG, MeIle, Nle, MeVal, |
| | | Cha, McGly, Aib |
| Aliphatic | A, V, L, I | b-Ala, Dpr, Aib, Aha, MeGly, |
| | | t-BuA, t-BuG, MeIle, Cha, Nle, |
| | | MeVal |
| Hydrophilic | | |
| Acidic | D, E | |
| Basic | H, K, R | Dpr, Orn, hArg, Phe(p-NH₂), Dbu, |
| | | Dab |
| Polar | C, Q, N, S. T | Cit, AcLys, MSO, bAla, hSer |
| Helix-Breaking | P, G | D-Pro and other D-amino acids |
| | | (in L-peptides) |

Other amino acid residues not specifically mentioned herein can be readily categorized based on their observed physical and chemical properties in light of the definitions provided herein.

While in most instances, the amino acids of the peptide mediators of RCT will be substituted with L-enantiomeric amino acids, the substitutions are not limited to L-enantiomeric amino acids. Thus, also included in the definition of “mutated” or “altered” forms are those situations where an L-amino acid is replaced with an identical D-amino acid (e.g., L-Arg→D-Arg) or with a D-amino acid of the same category or subcategory (e.g., L-Arg D-Lys), and vice versa. Indeed, in certain preferred embodiments that are suitable for oral administration to animal subjects, the peptides may advantageously be composed of at least one D-enantiomeric amino acid. Peptides containing such D-amino acids are thought to be more stable to degradation in the oral cavity, gut or serum than are peptides composed exclusively of L-amino acids.

As noted above, D-amino acids tend to disrupt the structure of α-helices when contained at internal positions with an α-helical L-peptide. Furthermore, it has been observed that certain mutated forms of the peptide mediators of RCT that are composed entirely of D-amino acids exhibit significantly lower LCAT activation in the assay described herein than identical peptides composed entirely of L-amino acids. As a consequence, D-amino acids are generally not used to substitute internal L-amino acids; D-amino acid substitutions are generally limited to 1-3 amino acid residues at the N-terminus and/or C-terminus of the peptide. In the case of small d-amino acid peptides this rule may not apply as multiple copies of the peptide might be associated to HDL or LDL to acquire the conformation necessary for the RCT.

As previously discussed, the amino acid Gly (G) generally acts as a helix-breaking residue when contained at internal positions of a peptide. Thus, although Gly (G) is generally considered to be a helix-breaking residue, Gly (G) can be used to substitute amino acids at internal positions of the peptide mediators of RCT. Preferably, only internal residues positioned within about ±1 helical turn of the center of the peptide (particularly for peptides composed of an even number of amino acids) are substituted with Gly (G). Additionally, it is preferred that only one internal amino acid residue in the peptide be substituted with Gly (G).

The native structure of ApoA-I contains eight helical units that are thought to act in concert to bind lipids (Nakagawa et al., 1985, J. Am. Chem. Soc. 107:7087-7092; Anantharamaiah et al., 1985, J. Biol. Chem. 260:10248-10262; Vanloo et al., 1991, J. Lipid Res. 32:1253-1264; Mendez et al., 1994, J. Clin. Invest. 94:1698-1705; Palgunari et al., 1996, Arterioscler. Thromb. Vasc. Biol. 16:328-338; Demoor et al., 1996, Eur. J. Biochem. 239:74-84). Thus, also included in the present invention are mediators of RCT comprised of dimers, trimers, tetramers and even higher order polymers (“multimers”) of the helical domains described herein. Such multimers may be in the form of tandem repeats, branched networks or combinations thereof. The peptide mediators of RCT may be directly attached to one another, separated by one or more linkers, or used independently to associate in multimeric stoichiometry with lipid (e.g., 2:1, 3:1, 4:1, 5:1, 6:1, 7:1, 8:1 of mediator:lipid, and possibly higher stoichiometric ratios).

The peptide mediators of RCT that comprise the multimers may comprise regions of the peptide sequence of ApoA-I, analogues of the ApoA-I sequence, mutated forms of ApoA-I, truncated or internally deleted forms of ApoA-I, extended forms of ApoA-I and/or combinations thereof. Truncated forms of the peptide mediators of RCT are obtained by deleting one or more amino acids from the N- and/or C-terminus of mediators of RCT. Internally deleted forms are obtained by deleting one or more amino acids from internal positions within the peptide mediators of RCT. The internal amino acid residues deleted may or may not be consecutive residues. Those of skill in the art will recognize that deleting an internal amino acid residue from a peptide mediators of RCT may cause the plane of the hydrophilic-hydrophobic interface of the helix to rotate at the point of the deletion. As such rotations can significantly alter the amphipathic properties of the resultant helix, in a preferred embodiment of the invention amino acid residues are deleted so as to substantially retain the alignment of the plane of the hydrophilic-hydrophobic interface along the entire long axis of the helix.

Linkers

The peptide mediators of RCT can be connected or linked in a head-to-tail fashion (i.e., N-terminus to C-terminus), a head-to-head fashion, (i.e., N-terminus to N-terminus), a tail-to-tail fashion (i.e., C-terminus to C-terminus), or combinations thereof. The linker LL can be any bifunctional molecule capable of covalently linking two peptides to one another. Thus, suitable linkers are bifunctional molecules in which the functional groups are capable of being covalently attached to the N- and/or C-terminus of a peptide. Functional groups suitable for attachment to the N- or C-terminus of peptides are well known in the art, as are suitable chemistries for effecting such covalent bond formation.

The linker may be flexible, rigid or semi-rigid, depending on the desired properties of the multimer. Suitable linkers include, for example, amino acid residues such as Pro or Gly or peptide segments containing from about 2 to about 5, 10, 15 or 20 or even more amino acids, bifunctional organic compounds such as H₂N(CH₂)ₙCOOH where n is an integer from 1 to 12, and the like. Examples of such linkers, as well as methods of making such linkers and peptides incorporating such linkers are well-known in the art (see, e.g., Hunig et al., 1974, Chem. Ber. 100:3039-3044; Basak et al., 1994, Bioconjug. Chem. 5(4):301-305).

Peptide and oligonucleotide linkers that can be selectively cleaved, as well as means for cleaving the linkers are well known and will be readily apparent to those of skill in the art. Suitable organic compound linkers that can be selectively cleaved will be apparent to those of skill in the art, and include those described, for example, in WO 94/08051, as well as the references cited therein.

Linkers of sufficient length and flexibility include, but are not limited to, Pro (P), Gly (G), Cys-Cys, H₂N—(CH₂)ₙ—COOH where n is 1 to 12, preferably 4 to 6; H₂N— aryl-COOH and carbohydrates.

Alternatively, as the native apolipoproteins permit cooperative binding between antiparallel helical segments, peptide linkers which correspond in primary sequence to the peptide segments connecting adjacent helices of the native apolipoproteins, including, for example, ApoA-I, ApoA-II, ApoA-IV, ApoC-I, ApoC-II, ApoC-III, ApoD, ApoE and ApoJ can be conveniently used to link the peptides. These sequences are well known in the art (see, e.g., Rosseneu et al., “Analysis of the Primary and of the Secondary Structure of the Apolipoproteins,” In: Structure and Function of Lipoproteins, Ch. 6, 159-183, CRC Press, Inc., 1992).

Other linkers which permit the formation of intermolecular hydrogen bonds or salt bridges between tandem repeats of antiparallel helical segments include peptide reverse turns such as β-turns and γ-turns, as well as organic molecules that mimic the structures of peptide β-turns and/or γ-turns. Generally, reverse turns are segments of peptide that reverse the direction of the polypeptide chain so as to allow a single polypeptide chain to adopt regions of antiparallel β-sheet or antiparallel α-helical structure. β-turns generally are composed of four amino acid residues and γ-turns are generally composed of three amino acid residues.

Alternatively, the linker (LL) may comprise an organic molecule or moiety that mimics the structure of a peptide β-turn or γ-turn. Such β-turn and/or γ-turn mimetic moieties, as well as methods for synthesizing peptides containing such moieties, are well known in the art, and include, among others, those described in Giannis and Kolter, 1993 Angew. Chem. Intl. Ed. Eng. 32:1244-1267; Kahn et al., 1988, J. Molecular Recognition 1:75-79; and Kahn et al., 1987, Tetrahedron Lett. 28:1623-1626.

The helical segments attached to a single linking moiety need not be attached via like termini. Indeed, in some embodiments the helical segments are attached to a single linking moiety so as to be arranged in an antiparallel fashion, i.e., some of the helices are attached via their N-termini, others via their C-termini.

The helical segments can be attached directly to the linking moiety, or may be spaced from the linking moiety by way of one or more bifunctional linkers (LL), as previously described.

The number of nodes in the network will generally depend on the total desired number of helical segments, and will typically be from about 1 to 2. Of course, it will be appreciated that for a given number of desired helical segments, networks having higher order linking moieties will have fewer nodes.

The networks may be of uniform order, i.e., networks in which all nodes are, for example, trifunctional or tetrafunctional linking moieties, or may be of mixed order, e.g., networks in which the nodes are mixtures of, for example, trifunctional and tetrafunctional linking moieties. Of course, it is to be understood that even in uniform order networks the linking moieties need not be identical. A tertiary order network may employ, for example, two, three, four or even more different trifunctional linking moieties.

Like the linear multimers, the helical segments comprising the branched network may be, but need not be, identical.

Analysis of Structure and Function

The structure and function of the mediators of RCT of the invention, including the multimeric forms described above, can be assayed in order to select active compounds. For example, the peptides or peptide analogues can be assayed for their ability to form α-helices, to bind lipids, to form complexes with lipids, to activate LCAT, and to promote cholesterol efflux, etc.

Methods and assays for analyzing the structure and/or function of the peptides are well-known in the art. Preferred methods are provided in the working examples, infra. For example, the circular dichroism (CD) and nuclear magnetic resonance (NMR) assays described, infra, can be used to analyze the structure of the peptides or peptide analogues—particularly the degree of helicity in the presence of lipids. The ability to bind lipids can be determined using the fluorescence spectroscopy assay described, infra. The ability of the peptides and/or peptide analogues to activate LCAT can be readily determined using the LCAT activation described, infra. The in vitro and in vivo assays described, infra, can be used to evaluate the half-life, distribution, cholesterol efflux and effects on RCT.

### Preferred Embodiment

The mediators of RCT of the invention can be further defined by way of preferred embodiments.

In one preferred embodiment, there is a molecule comprising an amino acid-based composition having three independent regions: an acidic region, an aromatic or lipophilic region, and a basic region. Thus, a trimeric peptide in accordance with this preferred embodiment, such as EFR, or erf or fre contains an acidic amino acid residue, an aromatic or lipophilic residue and a basic residue. The relative locations of the regions with respect to one another can vary between molecular mediators; the molecules mediate RCT regardless of the position of the three regions within each molecule. In mediators comprising a trimeric peptide, such as EFR or efr, the trimers may consist of natural D- or L- amino acids, amino acid analogs, and amino acid derivatives.

In another preferred embodiment, the aromatic region of the trimer may consist of nicotinic acid with an acidic or basic side chain(s).

In another preferred embodiment, the aromatic region of the trimer may consist of 4-phenyl phenylalanine.

In another preferred variation, the molecular mediators comprising an amino acid-based trimeric structure can optionally be capped by a lipophilic group(s) on the amino or carboxyl terminal at either end or both ends to improve the physicochemical properties of the molecular mediators of RCT and take advantage of the natural or active transport (absorption) system of fat or lipophilic materials into the body. The capping groups may be D or L enantiomers or non-enantiomeric molecules or groups. In preferred embodiments, the N-terminal capping groups are selected from the group consisting of acetyl, phenylacetyl, di-tert-butyl-4-hydroxy-phenyl, naphthyl, substituted naphthyl, f-MOC, biphenyl, substituted phenyl, substituted heterocycles, alkyl, aryl, substituted aryl, cycloalkyl, fused cycloalkyl, saturated heteroaryl, substituted saturated heteroaryl and the like. The C-terminal is preferably capped with an amine such as RNH₂ where R=di-tert-butyl-4-hydroxy-phenyl, naphthyl, substituted naphthyl, f-MOC, biphenyl, substituted phenyl, substituted heterocycles, alkyl, aryl, substituted aryl, cycloalkyl, fused cycloalkyl, saturated heteroaryl, substituted saturated heteroaryl, and the like.

In one preferred embodiment, the mediators of RCT of the invention are d from the group of peptides and peptide derivatives set forth in Table 3 below, n all of the peptides are capped with an acetyl group on the N-terminus and an amide on the C-terminus (unless otherwise specified):

TABLE 3

| | |
|---|---|
| SEQ | |
| ID NO | Sequence |

| | |
|---|---|
| 1 | YEFRDRMRTH |
| 2 | PVAEEFRDRMRTHVDSLRTQLAP |
| 3 | EEFRDRMRTHVDSLRTQLAP |
| 4 | FRDRMRTHVDSLRTQLAP |
| 5 | RDRMRTHVDSLRTQLAP |
| 6 | RTHVDSLRTQLAP |
| 7 | THVDSLRTQLAP |
| 8 | DSLRTQLAP |
| 9 | PVAEEFRDRMRTHV |
| 10 | PVAEEFRDRMR |
| 11 | PVAEEFRDRM |
| 12 | PVAEEFRDR |
| 13 | PVAEEF |
| 14 | PVAEE |
| 15 | MRTHVDSLRTQLAP |
| 16 | PVAEEFRDRMRTHVDSLR |
| 17 | WDKVKDF |
| 18 | SGRDYVSQFES |
| 19 | YLDEFQKKWKE |
| 20 | TRDFWDNLEKETDW |
| 21 | WDKVKDFANVYVDAVKD |
| 22 | PhCH2CO-YEFRDRMRTH |
| 23 | YEFRDRMRTH |
| 24 | Piv-YEFRDRMRTH |
| 25 | EFRDRMRTH |
| 26 | EFRDRMR |
| 27 | FRDRMRTH |
| 28 | yFRDRMRTh |
| 29 | eEFRDRMR |
| 30 | FrDRFRDr |
| 31 | EFRDRm |
| 32 | EFRDR |
| 33 | EFRD |
| 34 | EFR |
| 35 | rDRMRTh |
| 36 | dRMRTH |
| 37 | FrDRMRT |
| 38 | FRDRMR |
| 39 | FfRDRMr |
| 40 | FRDRM |
| 41 | YFRDRM |
| 42 | YFRDr |
| 43 | FRDRf |
| 44 | EFRDRMRTF |
| 45 | EFRDRf |
| 46 | FrDrFF |
| 47 | FrDrFY |
| 48 | FfDRFRDRf |
| 49 | mrDRFRDRm |
| 50 | FRDRFRDRF |
| 51 | FRDRMRDRM |
| 52 | MRDRFRDRM |
| 53 | RMRDRmr |
| 54 | FRDRMRDRF |
| 55 | EFRDRMRDRFE |
| 56 | FRdR |
| 57 | FRD |
| 58 | YFRD |
| 59 | frDRMRDRm |
| 60 | MRDRM |
| 61 | mRDRM |
| 62 | FRDRF |
| 63 | FRDRf |
| 64 | RMRDRMR |
| 65 | DRMRD |
| 66 | dRMRd |
| 67 | frDRMRDrF |
| 68 | RFEEFR |
| 69 | FRTRf |
| 70 | FRMRf |
| 71 | efRDRMRDRf |
| 72 | DRMRDF |
| 73 | yyyp-EFRDRMRTH |
| 74 | Yyp-EFRDRMRt |
| 75 | YyYpEFRDRMRt |
| 76 | EFRDRMRy |
| 77 | yyypEFRDRMR |
| 78 | YEFRDRm |
| 79 | yYYpEFRDRm |
| 80 | EFRDRy |
| 81 | EFRDy |
| 82 | yYYp-EFRD |
| 83 | efry |
| 84 | yYP-EFR |
| 85 | yYYp-EFRDr |
| 86 | erf |
| 87 | EEFRDR |
| 88 | EYR |
| 89 | E-(L-2-b-Naphtylalanine)-R |
| 90 | E-(L-1-b-Naphtylalanine)-R |
| 91 | E-BIP-R |
| 92 | H-EFR-OH |
| 93 | EFR-OH |
| 94 | H-EFR |
| 95 | RFE |
| 96 | efr |
| 97 | 2-Nap-EFR |
| 98 | yefr |
| 99 | 2-Nap-efry |
| 100 | Piv-efry |
| 101 | Fmoc-efry |
| 102 | 2-Nap-erf |
| 103 | 2-Nap-yefr |
| 104 | Piv-EFR |
| 105 | NA-EFR |
| 106 | 3,5-ditertiary-butyl-4-hydroxy-PhCO-E- |
| | (E-BIP-R-NH₂) |
| 107 | YWHVWQQDE |
| 108 | YQWDKVKDF |
| 109 | ENWDTLGSY |
| 110 | SGRDYVSQFES |
| 111 | VRQEMNKDLEEVKQKVY |
| 112 | YQMRESLAQRLY |
| 113 | TRDFWDNLEKETDWY |
| 114 | DEFQKKWKEY |
| 115 | WKEDVELYRQKV |
| 116 | YSLAQRLAELKSY |
| 117 | QESARQKLQELQY |
| 118 | yerf |
| 119 | rfe |
| 120 | Fmoc-EFR-OH |
| 121 | NA-yerf |
| 122 | NA-E-BIP-R |
| 123 | erfy |
| 124 | NA-erfy |
| 125 | 2-Nap-E-BIP-R |
| 126 | NA-efr |
| 127 | H-F-OH (L or D) |
| 128 | 3,5-ditertiary-butyl-4-hydroxy-PhCO-E-BIP-R |
| 129 | (1-Naphthyl)-L-alanine |
| 130 | (1-Naphthyl)-D-alanine |
| 131 | (2-Naphthyl)-L-alanine |
| 132 | (2-Naphthyl)-D-alanine |
| 133 | BIP-A |
| 134 | BIP-a |
| 135 | NA-fre |
| 136 | 1-Nap-erfy |
| 137 | 1-Nap-E-BIP-R |
| 138 | NA-yfre |
| 139 | yfre |
| 140 | NA-erf |
| 141 | 2-Nap-efry-OH |
| 142 | 3,5-ditertiary-butyl-4-hydroxy-PhCO-frey |
| 143 | NA-frey |
| 144 | NA-fr |
| 145 | e-BIP-r |
| 146 | e-bip-r |
| 147 | Isox-e-bip-r |
| 148 | 1-Nap-rfey |
| 149 | 1-Nap-frey |
| 150 | E-BIP-R-OH |
| 151 | 3,5-ditertiary-butyl-4-hydroxy-PhCO-erf |
| 152 | frey |
| 153 | rfey |
| 154 | 3,5-ditertiary-butyl-4-hydroxy-PhCO-fer |
| 155 | Isoxazole-refy |
| 156 | refy |
| 157 | NA-refy |
| 158 | 3,5-ditertiary-butyl-4-hydroxy-PhCO-refy |
| 159 | Isoxazole-fer |
| 160 | Isoxazole-yref |
| 161 | NA-yref |
| 162 | 3,5-ditertiary-butyl-4-hydroxy-PhCO-fery |
| 163 | 3,5-ditertiary-butyl-4-hydroxy-PhCO-fr |
| 164 | Isoxazole-fery |
| 165 | 3,5-ditertiary-butyl-4-hydroxy-PhCO-rfey |
| 166 | fery |
| 167 | 3,5-ditertiary-butyl-4-hydroxy-PhCO-rf |
| 168 | NA-yfer |
| 169 | Isoxazole-yfer |
| 170 | NA-fer |
| 171 | 3,5-ditertiary-butyl-4-hydroxy-PhCO-yfer |
| 172 | Isoxazole-rfe |
| 173 | 1-Nap-yfer |
| 174 | 1-Nap-rfe |
| 175 | NA-rfe |
| 176 | E-F(4-I)-R |

The abbreviations used for the D-enantiomers of the genetically encoded amino acids are lower-case equivalents of the one-letter symbols shown in Table 1. For example, “R” designates L-arginine and “r” designates D-arginine. Unless otherwise specified (eg. “OH”), the N-terminus is acetylated and the C-terminus is amidated.

PhAc denotes phenylacetylated.

Piv denotes pivolylated.

1-Nap & 2-Nap indicate naphthylic acid capped.

Fmoc denotes an N-terminus modified with 9-fluorenylmethyloxycarbonyl.

NA denotes nicotinic acid.

BIP denotes biphenylalanine.

Isoxazole denotes 5-methyl-isoxazole-3-carboxylic acid derivative

Amino acid substitutions need not be, and in certain embodiments preferably are not, restricted to the genetically encoded amino acids. Thus, in addition to the naturally occurring genetically encoded amino acids, amino acid residues in the peptide mediators of RCT may be substituted with naturally occurring non-encoded amino acids and synthetic amino acids.

Synthetic Methods

The peptides of the invention may be prepared using virtually any art-known technique for the preparation of peptides. For example, the peptides may be prepared using conventional step-wise solution or solid phase peptide syntheses, or recombinant DNA techniques.

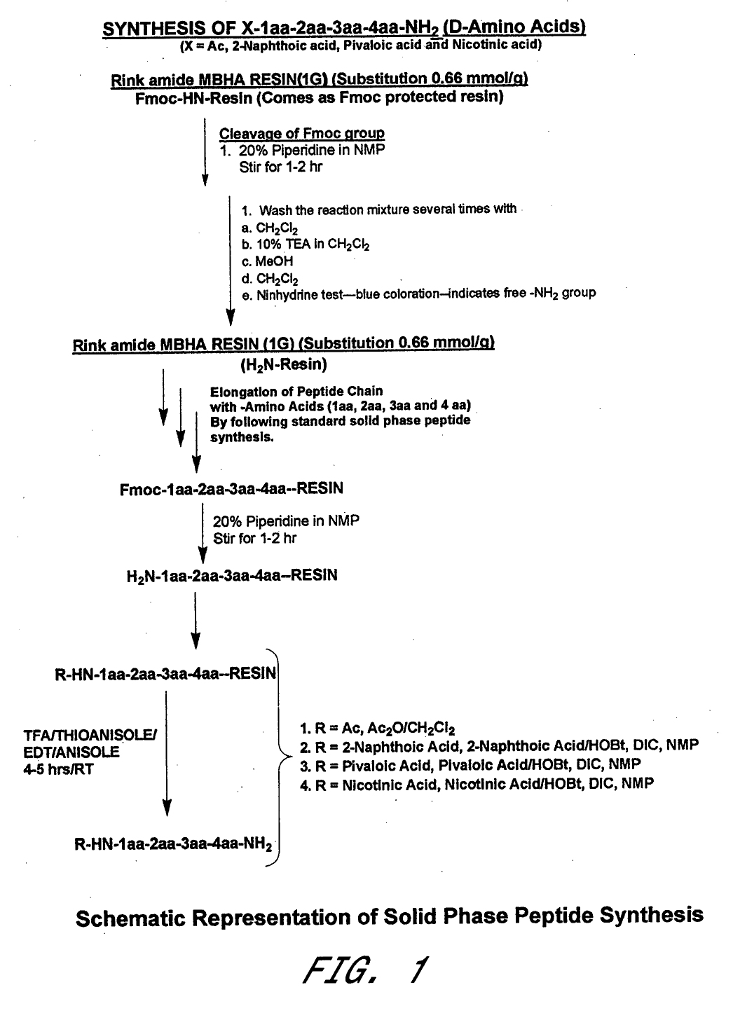

The peptide mediators of RCT may be prepared using conventional step-wise solution or solid phase synthesis (see, e.g., Chemical Approaches to the Synthesis of Peptides and Proteins, Williams et al., Eds., 1997, CRC Press, Boca Raton Fla., and references cited therein; Solid Phase Peptide Synthesis: A Practical Approach, Atherton & Sheppard, Eds., 1989, IRL Press, Oxford, England, and references cited therein). See FIG. 1.

In conventional solid-phase synthesis, attachment of the first amino acid entails chemically reacting its carboxyl-terminal (C-terminal) end with derivatized resin to form the carboxyl-terminal end of the oligopeptide. The alpha-amino end of the amino acid is typically blocked with a t-butoxy-carbonyl group (t-Boc) or with a 9-fluorenylmethyloxycarbonyl (F-Moc) group to prevent the amino group which could otherwise react from participating in the coupling reaction. The side chain groups of the amino acids, if reactive, are also blocked (or protected) by various benzyl-derived protecting groups in the form of ethers, thioethers, esters, and carbamates.

The next step and subsequent repetitive cycles involve deblocking the amino-terminal (N-terminal) resin-bound amino acid (or terminal residue of the peptide chain) to remove the alpha-amino blocking group, followed by chemical addition (coupling) of the next blocked amino acid. This process is repeated for however many cycles are necessary to synthesize the entire peptide chain of interest. After each of the coupling and deblocking steps, the resin-bound peptide is thoroughly washed to remove any residual reactants before proceeding to the next. The solid support particles facilitate removal of reagents at any given step as the resin and resin-bound peptide can be readily filtered and washed while being held in a column or device with porous openings.

Synthesized peptides may be released from the resin by acid catalysis (typically with hydrofluoric acid or trifluoroacetic acid), which cleaves the peptide from the resin leaving an amide or carboxyl group on its C-terminal amino acid. Acidolytic cleavage also serves to remove the protecting groups from the side chains of the amino acids in the synthesized peptide. Finished peptides can then be purified by any one of a variety of chromatography methods.

In accordance with a preferred embodiment, the peptides and peptide derivative mediators of RCT were synthesized by solid-phase synthesis methods with N^{a}-Fmoc chemistry. N^{a}-Fmoc protected amino acids and Rink amide MBHA resin and Wang resin were purchased from Novabiochem (San Diego, Calif.) or Chem-Impex Intl (Wood Dale, Ill.). Other chemicals and solvents were purchased from the following sources: trifluoroacetic acid (TFA), anisole, 1,2-ethanedithiol, thioanisole, piperidine, acetic anhydride, 2-Naphthoic acid and Pivaloic acid (Aldrich, Milwaukee, Wis.), HOBt and NMP (Chem-Impex Intl, Wood Dale, Ill.), dichloromethane, methanol and HPLC grade solvents from Fischer Scientific, Pittsburgh, Pa. The purity of the peptides was checked by LC/MS. The purification of the peptides was achieved using Preparative HPLC system (Agilent technologies, 1100 Series) on a C₁₈-bonded silica column (Tosoh Biospec preparative column, ODS-80TM, Dim: 21.5 mm×30 cm). The peptides were eluted with a gradient system [50% to 90% of B solvent (acetonitrile:water 60:40 with 0.1% TFA)].

All peptides were synthesized in a stepwise fashion via the solid-phase method, using Rink amide MBHA resin (0.5-0.66 mmol/g) or wang resin (1.2 mmol/g). The side chain's protecting groups were Arg (Pbf), Glu (OtBu) and Tyr (tBu). Each Fmoc-protected amino acid was coupled to this resin using a 1.5 to 3-fold excess of the protected amino acids. The coupling reagents were N-hydroxybenzotriazole (HOBt) and diisopropyl carbodiimide (DIC), and the coupling was monitored by Ninhydrin test. The Fmoc group were removed with 20% piperidine in NMP 30-60 minutes treatment and then successive washes with CH₂Cl₂, 10%TEA in CH₂Cl₂, Methanol and CH₂Cl₂. Coupling steps were followed by acetylation or with other capping groups as necessary.

A mixture of TFA, thioanisole, ethanedithiol and anisole (90:5:3:2, v/v) was used (4-5 hours at room temperature) to cleave the peptide from the peptide-resin and remove all of the side chain protecting groups. The crude peptide mixture was filtered from the sintered funnel, which was washed with TFA (2-3 times). The filtrate was concentrated into thick syrup and added into cold ether. The peptide precipitated as a white solid after keeping overnight in the freezer and centrifugation. The solution was decanted and the solid was washed thoroughly with ether. The resulting crude peptide was dissolved in buffer (acetonitrile:water 60:40 with 0.1% TFA) and dried. The crude peptide was purified by HPLC using preparative C-18 column (reverse phase) with a gradient system 50-90% B in 40 minutes [Buffer A: water containing 0.1% (v/v) TFA, Buffer B: Acetonitrile:water (60:40) containing 0.1% (v/v) TFA]. The pure fractions were concentrated over Speedvac. The yields varied from 5% to 20%.

Capped peptides synthesized as described above are shown in Table 4.

TABLE 4

| | |
|---|---|
| SEQ | |
| ID NO | Sequence |

| | |
|---|---|
| 34 | Ac-E-F-R-NH2 |
| 83 | Ac-e-f-r-y-NH₂ |
| 86 | Ac-e-r-f-NH2 |
| 91 | Ac-E-BIP-R-NH2 |
| 97 | 2-Nap-E-F-R-NH2 |
| 98 | Ac-y-e-f-r-NH2 |
| 99 | 2-Nap-e-f-r-yNH2 |
| 100 | Piv-e-f-r-y-NH2 |
| 101 | Fmoc-e-f-r-y-NH2 |
| 102 | 2-Nap-e-r-f-NH2 |
| 103 | 2-Nap-y-e-f-r-NH2 |
| 104 | Piv-E-F-R-NH2 |
| 105 | NA-E-F-R-NH2 |
| 106 | 3,5-ditertiary-butyl-4-hydroxy-PhCO-E- |
| | (E-BIP-R-NH2) |
| 118 | Ac-y-e-r-f-NH2 |
| 119 | Ac-r-f-e-NH2 |
| 120 | Fmoc-E-F-R-OH |
| 121 | NA-y-e-r-f-NH2 |
| 122 | NA-E-BIP-R-NH2 |
| 123 | Ac-e-r-f-y-NH2 |
| 124 | NA-e-r-f-y-NH2 |
| 126 | NA-e-f-r-NH2 |
| 125 | 2-Nap-E-BIP-R-NH2 |

Ac denotes acetylated.

Piv denotes pivolylated.

1-Nap & 2-Nap indicate naphthylic acid capped.

Fmoc denotes an N-terminus modified with 9-fluorenylmethyloxycarbonyl.

NA denotes nicotinic acid.

BIP denotes biphenylalanine.

Isoxazole denotes 5-methyl-isoxazole-3-carboxylic acid derivative.

Alternatively, the peptides of the invention may be prepared by way of segment condensation, i.e., the joining together of small constituent peptide chains to form a larger peptide chain, as described, for example, in Liu et al., 1996, Tetrahedron Lett. 37(7):933-936; Baca, et al., 1995, J. Am. Chem. Soc. 117:1881-1887; Tam et al., 1995, Int. J. Peptide Protein Res. 45:209-216; Schnolzer and Kent, 1992, Science 256:221-225; Liu and Tam, 1994, J. Am. Chem. Soc. 116(10):4149-4153; Liu and Tam, 1994, PNAS. USA 91:6584-6588; Yamashiro and Li, 1988, Int. J. Peptide Protein Res. 31:322-334; Nakagawa et al., 1985, J. Am Chem. Soc. 107:7087-7083; Nokihara et al., 1989, Peptides 1988:166-168; Kneib-Cordonnier et al., 1990, Int. J. Pept. Protein Res. 35:527-538; the disclosures of which are incorporated herein in their entirety by reference thereto). Other methods useful for synthesizing the peptides of the invention are described in Nakagawa et al., 1985, J. Am. Chem. Soc. 107:7087-7092.

For peptides produced by segment condensation, the coupling efficiency of the condensation step can be significantly increased by increasing the coupling time. Typically, increasing the coupling time results in increased racemization of the product (Sieber et al., 1970, Helv. Chim. Acta 53:2135-2150). However, since glycine lacks a chiral center it does not undergo racemization (proline residues, due to steric hindrance, also undergo little or no racemization at long coupling times). Thus, embodiments containing internal glycine residues can be synthesized in bulk in high yield via segment condensation by synthesizing constituent segments which take advantage of the fact that glycine residues do not undergo racemezation. Thus, embodiments containing internal glycine residues provide significant synthetic advantages for large-scale bulk preparation.

Mediators of RCT containing N- and/or C-terminal blocking groups can be prepared using standard techniques of organic chemistry. For example, methods for acylating the N-terminus of a peptide or amidating or esterifying the C-terminus of a peptide are well-known in the art. Modes of carrying other modifications at the N- and/or C-terminus will be apparent to those of skill in the art, as will modes of protecting any side-chain functionalities as may be necessary to attach terminal blocking groups.

Pharmaceutically acceptable salts (counter ions) can be conveniently prepared by ion-exchange chromatography or other methods as are well known in the art.

Compounds of the invention which are in the form of tandem multimers can be conveniently synthesized by adding the linker(s) to the peptide chain at the appropriate step in the synthesis. Alternatively, the helical segments can be synthesized and each segment reacted with the linker. Of course, the actual method of synthesis will depend on the composition of the linker. Suitable protecting schemes and chemistries are well known, and will be apparent to those of skill in the art.

Compounds of the invention which are in the form of branched networks can be conveniently synthesized using the trimeric and tetrameric resins and chemistries described in Tam, 1988, PNAS USA 85:5409-5413 and Demoor et al., 1996, Eur. J. Biochem. 239:74-84. Modifying the synthetic resins and strategies to synthesize branched networks of higher or lower order, or which contain combinations of different peptide helical segments, is well within the capabilities of those of skill in the art of peptide chemistry and/or organic chemistry.

Formation of disulfide linkages, if desired, is generally conducted in the presence of mild oxidizing agents. Chemical oxidizing agents may be used, or the compounds may simply be exposed to atmospheric oxygen to effect these linkages. Various methods are known in the art, including those described, for example, by Tam et al., 1979, Synthesis 955-957; Stewart et al., 1984, Solid Phase Peptide Synthesis, 2d Ed., Pierce Chemical Company Rockford, Ill.; Ahmed et al., 1975, J. Biol. Chem. 250:8477-8482; and Pennington et al., 1991 Peptides 1990:164-166, Giralt and Andreu, Eds., ESCOM Leiden, The Netherlands. An additional alternative is described by Kamber et al., 1980, Helv. Chim. Acta 99-915. A method conducted on solid supports is described by Albericio, 1985, Int. J. Peptide Protein Res. 26:92-97. Any of these methods may be used to form disulfide linkages in the peptides of the invention. Additional chemically synthesized amino acid-derived compounds are shown in the following Table 5.

TABLE 5

| COM- | | MOL. | MOL. |
|---|---|---|---|
| POUND # | SEQUENCE | FORMULA | WEIGHT |
| 1 | Ac-E-F-R-NH₂ | C₂₂H₃₃N₇O₆ | 491.55 |
| 2 | Ac-e-r-f-NH₂ | C₂₂H₃₃N₇O₆ | 491.55 |
| 3 | Ac-E-BIP-R-NH₂ | C₂₈H₃₇N₇O₆ | 567.64 |
| 4 | Ac-e-f-r-y-NH₂ | C₃₁H₄₂N₈O₈ | 654.72 |
| 5 | 2-Naph-E-F-R-NH₂ | C₃₁H₃₇N₇O₆ | 603.68 |
| 6 | Ac-y-e-f-r-NH₂ | C₃₁H₄₂N₈O₈ | 654.72 |
| 7 | 2-Nap-e-f-r-y-NH₂ | C₄₀H₄₆N₈O₈ | 766.85 |
| 8 | Piv-e-f-r-y-NH₂ | C₃₄H₄₈N₈O₈ | 696.80 |
| 9 | Fmoc-e-f-r-y-NH₂ | C₄₄H₅₀N₈O₉ | 834.93 |
| 10 | 2-Nap-e-r-f-NH₂ | C₃₁H₃₇N₇O₆ | 603.68 |
| 11 | 2-Nap-y-e-f-r-NH₂ | C₄₀H₄₆N₈O₈ | 766.85 |
| 12 | Piv-E-F-R-NH₂ | C₂₅H₃₉N₇O₆ | 533.63 |
| 13 | NA-E-F-R-NH₂ | C₂₆H₃₄N₈O₆ | 554.61 |
| 14 | Ac-y-e-r-f-NH₂ | C₃₁H₄₂N₈O₈ | 654.72 |
| 15 | Ac-r-f-e-NH₂ | C₂₂H₃₃N₇O₆ | 491.55 |
| 16 | Fmoc-E-F-R-OH | C₃₅H₄₀N₆O₈ | 672.74 |
| 17 | NA-y-e-r-f-NH₂ | C₃₅H₄₃N₉O₈ | 717.78 |
| 18 | NA-E-BIP-R-NH₂ | C₃₂H₃₈N₈O₆ | 630.70 |
| 19 | NA-e-r-f-y-NH₂ | C₃₅H₄₃N₉O₈ | 717.78 |
| 20 | 2-Nap-E-BIP-R-NH₂ | C₃₇H₄₁N₇O₆ | 679.77 |
| 21 | NA-e-f-r-NH₂ | C₂₆H₃₄N₈O₆ | 554.61 |
| 22 | 3,5-ditertiary-butyl-4-hydroxy- | C₄₁H₅₅N₇O₇ | 757.93 |
| | PhCO-E-BIP-R-NH₂ | | |
| 23 | 1-Nap-L-Alanine | C₁₃H₁₃NO₂ | 215.25 |
| 24 | 1-Nap-D-Alanine | C₁₃H₁₃NO₂ | 215.25 |
| 25 | 2-Nap-L-Alanine | C₁₃H₁₃NO₂ | 215.25 |
| 26 | 2-Nap-D-Alanine | C₁₃H₁₃NO₂ | 215.25 |
| 27 | L-Biphenylalanine | C₁₅H₁₅NO₂ | 241.3 |
| 28 | D-Biphenylalanine | C₁₅H₁₅NO₂ | 241.3 |
| 29 | NA-f-r-e-NH₂ | C₂₆H₃₄N₈O₆ | 554.61 |
| 30 | 1-Nap-e-r-f-y-NH₂ | C₄₀H₄₆N₈O₈ | 766.84 |
| 31 | 1-Nap-E-BIP-R-NH₂ | C₃₇H₄₁N₇O₆ | 679.77 |
| 32 | NA-y-f-r-e-NH₂ | C₃₅H₄₃N₉O₈ | 717.78 |
| 33 | Ac-y-f-r-e-NH₂ | C₃₁H₄₂N₈O₈ | 654.72 |
| 34 | NA-e-r-f-NH₂ | C₂₆H₃₄N₈O₆ | 554.61 |
| 35 | 2-Nap-e-f-r-y-OH | C₄₀H₄₅N₇O₉ | 767.84 |
| 36 | 3,5-ditertiary-butyl-4-hydroxy- | C₄₄H₆₀N₈O₉ | 845.01 |
| | PhCO-f-r-e-y-NH₂ | | |
| 37 | NA-f-r-e-y-NH₂ | C₃₅H₄₃N9₈O₈ | 717.78 |
| 38 | NA-f-r-NH₂ | C₂₁H₂₇N₇O₃ | 425.49 |
| 39 | Ac-e-BIP-r-NH₂ | C₂₈H₃₇N₇O₆ | 567.64 |
| 40 | Ac-e-bip-r-NH₂ | C₂₈H₃₇N₇O₆ | 567.64 |
| 41 | Isoxazole-e-bip-r-NH₂ | C₃₁H₃₈N₈O₇ | 634.69 |
| 42 | 1-Nap-r-f-e-y-NH₂ | C₄₀H₄₆N₈O₈ | 766.84 |
| 43 | 1-Nap-f-r-e-y-NH₂ | C₄₀H₄₆N₈O₈ | 766.84 |
| 44 | Ac-E-BIP-R-OH | C₃₂H₃₈N₈O₆ | 568.63 |
| 45 | 3,5-ditertiary-butyl-4-hydroxy- | C₃₅H₅₁N₇O₇ | 681.82 |
| | PhCO-e-r-f-NH₂ | | |
| 46 | Ac-f-r-e-y-NH₂ | C₃₁H₄₂N₈O₈ | 654.72 |
| 47 | Ac-r-f-e-y-NH₂ | C₃₁H₄₂N₈O₈ | 654.72 |
| 48 | 3,5-ditertiary-butyl-4-hydroxy- | C₃₅H₅₁N₇O₇ | 681.82 |
| | PhCO-f-e-r-NH₂ | | |
| 49 | Isoxazole-r-e-f-y-NH₂ | C₃₄H₄₃N₉O₉ | 721.77 |
| 50 | Ac-r-e-f-y-NH₂ | C₃₁H₄₂N₈O₈ | 654.72 |
| 51 | NA-r-e-f-y-NH₂ | C₃₅H₄₃N₉O₈ | 717.78 |
| 52 | 3,5-ditertiary-butyl-4-hydroxy- | C₄₄H₆₀N₈O₉ | 845.01 |
| | PhCO-r-e-f-y-NH₂ | | |
| 53 | Isaxazole-f-e-r-NH₂ | C₃₅H₅₁N₇O₇ | 558.59 |
| 54 | Isoxazole-y-r-e-f-NH₂ | C₃₄H₄₃N₉O₉ | 721.77 |
| 55 | NA-y-r-e-f-NH₂ | C₃₅H₄₃N₉O₈ | 717.78 |
| 56 | 3,5-ditertiary-butyl-4-hydroxy- | C₄₄H₆₀N₈O₉ | 845.01 |
| | PhCO-f-r-e-y-NH₂ | | |
| 57 | 3,5-ditertiary-butyl-4-hydroxy- | C₃₀H₄₄N₆O₄ | 552.72 |
| | PhCO-f-r-NH₂ | | |
| 58 | Isoxazole-f-e-r-y-NH₂ | C₃₄H₄₃N₉O₉ | 721.77 |
| 59 | 3,5-ditertiary-butyl-4-hydroxy- | C₄₄H₆₀N₈O₉ | 845.01 |
| | PhCO-r-f-e-y-NH₂ | | |
| 60 | Ac-f-e-r-y-NH₂ | C₃₁H₄₂N₈O₈ | 654.72 |
| 61 | 3,5-ditertiary-butyl-4-hydroxy- | C₃₀H₄₄N₆O₄ | 552.72 |
| | PhCO-f-r-NH₂ | | |
| 62 | NA-y-f-e-r-NH₂ | C₃₅H₄₃N₉O₈ | 717.78 |
| 63 | Isaxazole-y-f-e-r-NH₂ | C₃₄H₄₃N₉O₉ | 721.77 |
| 64 | NA-f-e-r-NH₂ | C₂₆H₃₄N₈O₆ | 554.61 |
| 65 | 3,5-ditertiary-butyl-4-hydroxy- | C₄₄H₆₀N₈O₉ | 845.01 |
| | PhCO-y-f-e-r-NH₂ | | |
| 66 | Isoxazole-r-f-e-NH₂ | C₃₅H₅₁N₇O₇ | 558.59 |
| 67 | 1-Nap-y-f-e-r-NH₂ | C₄₀H₄₆N₈O₈ | 766.84 |
| 68 | 1-Nap-r-f-e-NH₂ | C₃₁H₃₇N₇O₆ | 603.68 |
| 69 | NA-r-f-e-NH₂ | C₂₆H₃₄N₈O₆ | 554.61 |
| 70 | Ac-E-F(4-I)-R-NH₂ | C₂₂H₃₂IN₇O₆ | 617.44 |
| 71 | NA-R-BIP-E-NH₂ | C₃₂H₃₈N₈O₆ | 630.70 |
| 72 | 1-Nap-R-BIP-E-NH₂ | C₃₇H₄₁N₇O₆ | 679.77 |
| 73 | Ac-R-BIP-E-NH₂ | C₂₈H₃₇N₇O₆ | 567.64 |
| 74 | Ac-E-BIP-K-NH₂ | C₂₈H₃₇N₅O₆ | 539.63 |
| 75 | Ac-D-BIP-K-NH₂ | C₂₇H₃₅N₅O₆ | 525.60 |
| 76 | Ac-d-BIP-r-NH₂ | C₂₇H₃₇N₅O₆ | 553.62 |
| 77 | Ac-e-bip-k-NH₂ | C₂₈H₃₇N₅O₆ | 539.63 |
| 78 | Ac-d-bip-k-NH₂ | C₂₇H₃₅N₅O₆ | 525.60 |
| 79 | Ac-d-bip-r-NH₂ | C₂₇H₃₇N₅O₆ | 553.62 |
| 80 | Ac-E-R-BIP-NH₂ | C₂₈H₃₇N₇O₆ | 567.64 |
| 81 | 2-Pyrazine-CO-E-R-BIP-NH₂ | C₃₁H₃₇N₉O₆ | 631.60 |
| 82 | Piperonyl-CO-E-R-BIP-NH₂ | C₃₄H₃₉N₇O₈ | 673.72 |
| 83 | Ac-E-bip-R-NH₂ | C₂₈H₃₇N₇O₆ | 567.64 |
| 84 | Ac-e-f(4-I)-r-NH₂ | C₂₂H₃₂IN₇O₆ | 617.44 |
| 85 | Glutaric-BIP-R-NH₂ | C₂₆H₃₄N₆O₅ | 510.59 |
| 86 | Glutaric-bip-r-NH₂ | C₂₆H₃₄N₆O₅ | 510.59 |
| 87 | Ac-E-BIP-Agmatine | C₂₇H₃₆N₆O₅ | 524.62 |
| 88 | Ac-e-bip-Agmatine | C₂₇H₃₆N₆O₅ | 524.62 |
| 89 | Ac-R-BIP-GABA | C₂₇H₃₆N₆O₅ | 524.62 |
| 90 | Ac-r-bip-GABA | C₂₇H₃₆N₆O₅ | 524.62 |
| 91 | Agmatine-BIP-E-NH₂ | C₂₅H₃₂N₆O₅ | 496.56 |
| 92 | Agmatine-bip-e-NH₂ | C₂₅H₃₂N₆O₅ | 496.56 |
| 93 | Ac-e-bip(4-tBu)-r-NH₂ | C₃₂H₄₅N₇O₆ | 673.75 |
| 94 | Ac-E-BIP(4-tBu)-R-NH₂ | C₃₂H₄₅N₇O₆ | 673.75 |
| 95 | Glutaric-BIP-K-NH₂ | C₂₆H₃₄N₄O₅ | 482.58 |
| 96 | Glutaric-bip-k-NH₂ | C₂₆H₃₄N₄O₅ | 482.58 |

The abbreviations used for the D-enantiomers of the genetically encoded amino acids are lower-case equivalents of the one-letter symbols shown in Table 1. For example “R” designates L-arginine and “r” designates D-arginine.

Ac denotes acetylated.

Piv denotes pivolylated.

1-Nap & 2-Nap indicate naphthylic acid capped.

Fmoc denotes an N-terminus modified with 9-fluorenylmethyloxycarbonyl.

NA denotes nicotinic acid.

BIP denotes biphenylalanine.

Isoxazole denotes 5-methyl-isoxazole-3-carboxylic acid derivative

If the peptide is composed entirely of gene-encoded amino acids, or a portion of it is so composed, the peptide or the relevant portion may also be synthesized using conventional recombinant genetic engineering techniques.

For recombinant production, a polynucleotide sequence encoding the peptide is inserted into an appropriate expression vehicle, i.e., a vector which contains the necessary elements for the transcription and translation of the inserted coding sequence, or in the case of an RNA viral vector, the necessary elements for replication and translation. The expression vehicle is then transfected into a suitable target cell which will express the peptide. Depending on the expression system used, the expressed peptide is then isolated by procedures well-established in the art. Methods for recombinant protein and peptide production are well known in the art (see, e.g., Sambrook et al., 1989, Molecular Cloning A Laboratory Manual, Cold Spring Harbor Laboratory, N.Y.; and Ausubel et al., 1989, Current Protocols in Molecular Biology, Greene Publishing Associates and Wiley Interscience, N.Y. each of which is incorporated by reference herein in its entirety.)

To increase efficiency of production, the polynucleotide can be designed to encode multiple units of the peptide separated by enzymatic cleavage sites either homopolymers (repeating peptide units) or heteropolymers (different peptides strung together) can be engineered in this way. The resulting polypeptide can be cleaved (e.g., by treatment with the appropriate enzyme) in order to recover the peptide units. This can increase the yield of peptides driven by a single promoter. In a preferred embodiment, a polycistronic polynucleotide can be designed so that a single MRNA is transcribed which encodes multiple peptides (i.e., homopolymers or heteropolymers) each coding region operatively linked to a cap-independent translation control sequence; e.g., an internal ribosome entry site (IRES). When used in appropriate viral expression systems, the translation of each peptide encoded by the MRNA is directed internally in the transcript; e.g., by the IRES. Thus, the polycistronic construct directs the transcription of a single, large polycistronic MRNA which, in turn, directs the translation of multiple, individual peptides. This approach eliminates the production and enzymatic processing of polyproteins and may significantly increase yield of peptide driven by a single promoter.

A variety of host-expression vector systems may be utilized to express the peptides described herein. These include, but are not limited to, microorganisms such as bacteria transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; or animal cell systems.

The expression elements of the expression systems vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage X, plac, ptrp, ptac (ptrp-lac hybrid promoter) and the like may be used; when cloning in insect cell systems, promoters such as the baculovirus polyhedron promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5 K promoter) may be used; when generating cell lines that contain multiple copies of expression product, SV40-, BPV- and EBV-based vectors may be used with an appropriate selectable marker.

In cases where plant expression vectors are used, the expression of sequences encoding the peptides of the invention may be driven by any of a number of promoters. For example, viral promoters such as the 35S RNA and 19S RNA promoters of CaMV (Brisson et al., 1984, Nature 310:511-514), or the coat protein promoter of TMV (Takamatsu et al., 1987, EMBO J. 6:307-311) may be used; alternatively, plant promoters such as the small subunit of RUBISCO (Coruzzi et al., 1984, EMBO J. 3:1671-1680; Broglie et al., 1984, Science 224:838-843) or heat shock promoters, e.g., soybean hsp17.5-E or hsp17.3-B (Gurley et al., 1986, Mol. Cell. Biol. 6:559-565) may be used. These constructs can be introduced into plant cells using Ti plasmids, Ri plasmids, plant virus vectors, direct DNA transformation, microinjection, electroporation, etc. For reviews of such techniques see, e.g., Weissbach & Weissbach, 1988, Methods for Plant Molecular Biology, Academic Press, NY, Section VIII, pp. 421-463; and Grierson & Corey, 1988, Plant Molecular Biology, 2d Ed., Blackie, London, Ch. 7-9.

In one insect expression system that may be used to produce the peptides of the invention, Autographa californica, nuclear polyhidrosis virus (AcNPV) is used as a vector to express the foreign genes. The virus grows in Spodoptera frugiperda cells. A coding sequence may be cloned into non-essential regions (for example the polyhedron gene) of the virus and placed under control of an AcNPV promoter (for example, the polyhedron promoter). Successful insertion of a coding sequence will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed (e.g., see Smith et al., 1983, J. Virol. 46:584; Smith, U.S. Pat. No. 4,215,051). Further examples of this expression system may be found in Current Protocols in Molecular Biology, Vol. 2, Ausubel et al., eds., Greene Publish. Assoc. & Wiley Interscience.

In mammalian host cells, a number of viral based expression systems may be utilized. In cases where an adenovirus is used as an expression vector, a coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing peptide in infected hosts. (e.g., See Logan & Shenk, 1984, PNAS USA 81:3655-3659). Alternatively, the vaccinia 7.5 K promoter may be used, (see, e.g., Mackett et al., 1982, PNAS USA 79:7415-7419; Mackett et al., 1984, J. Virol. 49:857-864; Panicali et al., 1982, PNAS USA 79:4927-4931).

Other expression systems for producing the peptides of the invention will be apparent to those having skill in the art.

Purification of Peptides

The peptides of the invention can be purified by art-known techniques such as reverse phase high performance liquid chromatography (e.g., the crude peptides synthesized by solid-phase synthesis methods with Na-Fmoc chemistry, described above were purified by reverse phase HPLC using preparative C-18 column), ion exchange chromatography, gel electrophoresis, affinity chromatography and the like. The actual conditions used to purify a particular peptide will depend, in part, on synthesis strategy and on factors such as net charge, hydrophobicity, hydrophilicity, etc., and will be apparent to those having skill in the art. Multimeric branched peptides can be purified, e.g., by ion exchange or size exclusion chromatography.

For affinity chromatography purification, any antibody which specifically binds the peptide may be used. For the production of antibodies, various host animals, including but not limited to rabbits, mice, rats, etc., may be immunized by injection with a peptide. The peptide may be attached to a suitable carrier, such as BSA, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette-Guerin) and Corynebacterium parvum.

Monoclonal antibodies to a peptide may be prepared using any technique which provides for the production of antibody molecules by continuous cell lines in culture. These include but are not limited to the hybridoma technique originally described by Kohler and Milstein, 1975, Nature 256:495-497, or Kaprowski, U.S. Pat. No. 4,376,110 which is incorporated by reference herein; the human B-cell hybridoma technique) Kosbor et al., 1983, Immunology Today 4:72; Cote et al., 1983, PNAS USA 80:2026-2030); and the EBV-hybridoma technique (Cole et al., 1985, Monoclonal Antibodies and Cancer Therapy, Alan R. Liss, Inc., pp. 77-96). In addition, techniques developed for the production of “chimeric antibodies” Morrison et al., 1984, PNAS USA 81:6851-6855; Neuberger et al., 1984, Nature 312:604-608; Takeda et al., 1985, Nature 314:452-454, Boss, U.S. Pat. No. 4,816,397; Cabilly, U.S. Pat. No. 4,816,567; which are incorporated by reference herein) by splicing the genes from a mouse antibody molecule of appropriate antigen specificity together with genes from a human antibody molecule of appropriate biological activity can be used. Or “humanized” antibodies can be prepared (see, e.g., Queen, U.S. Pat. No. 5,585,089 which is incorporated by reference herein). Alternatively, techniques described for the production of single chain antibodies (U.S. Pat. No. 4,946,778) can be adapted to produce peptide-specific single chain antibodies.

Antibody fragments which contain deletions of specific binding sites may be generated by known techniques. For example, such fragments include but are not limited to F(ab′)₂ fragments, which can be produced by pepsin digestion of the antibody molecule and Fab fragments, which can be generated by reducing the disulfide bridges of the F(ab′)₂ fragments. Alternatively, Fab expression libraries may be constructed (Huse et al., 1989, Science 246:1275-1281) to allow rapid and easy identification of monoclonal Fab fragments with the desired specificity for the peptide of interest.

The antibody or antibody fragment specific for the desired peptide can be attached, for example, to agarose, and the antibody-agarose complex is used in immunochromatography to purify peptides of the invention. See, Scopes, 1984, Protein Purification: Principles and Practice, Springer-Verlag New York, Inc., N.Y., Livingstone, 1974, Methods In Enzymology: Immunoaffinity Chromatography of Proteins 34:723-731.

### Pharmaceutical Formulations and Methods of Treatment

The mediators of RCT of the invention can be used to treat any disorder in animals, especially mammals including humans, for which lowering serum cholesterol is beneficial, including without limitation conditions in which increasing serum HDL concentration, activating LCAT, and promoting cholesterol efflux and RCT is beneficial. Such conditions include, but are not limited to hyperlipidemia, and especially hypercholesterolemia, and cardiovascular disease such as atherosclerosis (including treatment and prevention of atherosclerosis) and coronary artery disease; restenosis (e.g., preventing or treating atherosclerotic plaques which develop as a consequence of medical procedures such as balloon angioplasty); and other disorders, such as ischemia, and endotoxemia, which often results in septic shock.

The mediators of RCT can be used alone or in combination therapy with other drugs used to treat the foregoing conditions. Such therapies include, but are not limited to simultaneous or sequential administration of the drugs involved.

For example, in the treatment of hypercholesterolemia or atherosclerosis, the formulations of molecular mediators of RCT can be administered with any one or more of the cholesterol lowering therapies currently in use; e.g., bile-acid resins, niacin, and/or statins. Such a combined treatment regimen may produce particularly beneficial therapeutic effects since each drug acts on a different target in cholesterol synthesis and transport; i.e., bile-acid resins affect cholesterol recycling, the chylomicron and LDL population; niacin primarily affects the VLDL and LDL population; the statins inhibit cholesterol synthesis, decreasing the LDL population (and perhaps increasing LDL receptor expression); whereas the mediators of RCT affect RCT, increase HDL, increase LCAT activity and promote cholesterol efflux.

The mediators of RCT may be used in conjunction with fibrates to treat hyperlipidemia, hypercholesterolemia and/or cardiovascular disease such as atherosclerosis.

The mediators of RCT of the invention can be used in combination with the anti-microbials and anti-inflammatory agents currently used to treat septic shock induced by endotoxin.

The mediators of RCT of the invention can be formulated as peptide-based compositions or as peptide-lipid complexes which can be administered to subjects in a variety of ways, preferrably via oral administration, to deliver the mediators of RCT to the circulation. Exemplary formulations and treatment regimens are described below.

In another preferred embodiment of the present invention, methods are provided for ameliorating and/or preventing one or more symptoms of hypercholesterolemia and/or atherosclerosis. The methods preferably involve administering to an organism, preferably a mammal, more preferably a human one or more of the peptides of this invention (or mimetics of such peptides). The peptide(s) can be administered, as described herein, according to any of a number of standard methods including, but not limited to injection, suppository, nasal spray, time-release implant, transdermal patch, and the like. In one particularly preferred embodiment, the peptide(s) are administered orally (e.g. as a syrup, capsule, or tablet).

The methods involve the administration of a single polypeptide of this invention or the administration of two or more different polypeptides. The polypeptides can be provided as monomers or in dimeric, oligomeric or polymeric forms. In certain embodiments, the multimeric forms may comprise associated monomers (e.g. ionically or hydrophobically linked) while certain other multimeric forms comprise covalently linked monomers (directly linked or through a linker).

While the invention is described with respect to use in humans, it is also suitable for animal, e.g. veterinary use. Thus preferred organisms include, but are not limited to humans, non-human primates, canines, equines, felines, porcines, ungulates, largomorphs, and the like.

The methods of this invention are not limited to humans or non-human animals showing one or more symptom(s) of hypercholesterolemia and/or atherosclerosis (e.g., hypertension, plaque formation and rupture, reduction in clinical events such as heart attack, angina, or stroke, high levels of low density lipoprotein, high levels of very low density lipoprotein, or inflammatory proteins, etc.), but are useful in a prophylactic context. Thus, the peptides of this invention (or mimetics thereof) may be administered to organisms to prevent the onset/development of one or more symptoms of hypercholesterolemia and/or atherosclerosis. Particularly preferred subjects in this context are subjects showing one or more risk factors for atherosclerosis (e.g., family history, hypertension, obesity, high alcohol consumption, smoking, high blood cholesterol, high blood triglycerides, elevated blood LDL, VLDL, IDL, or low HDL, diabetes, or a family history of diabetes, high blood lipids, heart attack, angina or stroke, etc.).

In one preferred embodiment, the peptide mediators of RCT can be synthesized or manufactured using any technique described in earlier sections pertaining to synthesis and purification of the mediators of RCT. Stable preparations which have a long shelf life may be made by lyophilizing the peptides-either to prepare bulk for reformulation, or to prepare individual aliquots or dosage units which can be reconstituted by rehydration with sterile water or an appropriate sterile buffered solution prior to administration to a subject.

In another preferred embodiment, the mediators of RCT may be formulated and administered in a peptide-lipid complex. This approach has some advantages since the complex should have an increased half-life in the circulation, particularly when the complex has a similar size and density to HDL, and especially the pre-β-1 or pre-β-2 HDL populations. The peptide-lipid complexes can conveniently be prepared by any of a number of methods described below. Stable preparations having a long shelf life may be made by lyophilization—the co-lyophilization procedure described below being the preferred approach. The lyophilized peptide-lipid complexes can be used to prepare bulk for pharmaceutical reformulation, or to prepare individual aliquots or dosage units which can be reconstituted by rehydration with sterile water or an appropriate buffered solution prior to administration to a subject.

A variety of methods well known to those skilled in the art can be used to prepare the peptide-lipid vesicles or complexes. To this end, a number of available techniques for preparing liposomes or proteoliposomes may be used. For example, the peptide can be cosonicated (using a bath or probe sonicator) with appropriate lipids to form complexes. Alternatively the peptide can be combined with preformed lipid vesicles resulting in the spontaneous formation of peptide-lipid complexes. In yet another alternative, the peptide-lipid complexes can be formed by a detergent dialysis method; e.g., a mixture of the peptide, lipid and detergent is dialyzed to remove the detergent and reconstitute or form peptide-lipid complexes (e.g., see Jonas et al., 1986, Methods in Enzymol. 128:553-582).

While the foregoing approaches are feasible, each method presents its own peculiar production problems in terms of cost, yield, reproducibility and safety. In accordance with one preferred method, the peptide and lipid are combined in a solvent system which co-solubilizes each ingredient and can be completely removed by lyophilization. To this end, solvent pairs should be carefully selected to ensure co-solubility of both the amphipathic peptide and the lipid. In one embodiment, the protein(s), peptide(s) or derivatives/analogs thereof, to be incorporated into the particles can be dissolved in an aqueous or organic solvent or mixture of solvents (solvent 1). The (phospho)lipid component is dissolved in an aqueous or organic solvent or mixture of solvents (solvent 2) which is miscible with solvent 1, and the two solutions are mixed. Alternatively, the peptide and lipid can be incorporated into a co-solvent system; i.e., a mixture of the miscible solvents. A suitable proportion of peptide (protein) to lipids is first determined empirically so that the resulting complexes possess the appropriate physical and chemical properties; i.e., usually (but not necessarily) similar in size to HDL. The resulting mixture is frozen and lyophilized to dryness. Sometimes an additional solvent must be added to the mixture to facilitate lyophilization. This lyophilized product can be stored for long periods and will remain stable.

The lyophilized product can be reconstituted in order to obtain a solution or suspension of the peptide-lipid complex. To this end, the lyophilized powder may be rehydrated with an aqueous solution to a suitable volume (often 5 mgs peptide/ml which is convenient for intravenous injection). In a preferred embodiment the lyophilized powder is rehydrated with phosphate buffered saline or a physiological saline solution. The mixture may have to be agitated or vortexed to facilitate rehydration, and in most cases, the reconstitution step should be conducted at a temperature equal to or greater than the phase transition temperature of the lipid component of the complexes. Within minutes, a clear preparation of reconstituted lipid-protein complexes results.

An aliquot of the resulting reconstituted preparation can be characterized to confirm that the complexes in the preparation have the desired size distribution; e.g., the size distribution of HDL. Gel filtration chromatography can be used to this end. For example, a Pharmacia Superose 6 FPLC gel filtration chromatography system can be used. The buffer used contains 150 mM NaCl in 50 mM phosphate buffer, pH 7.4. A typical sample volume is 20 to 200 microliters of complexes containing 5 mgs peptide/ml. The column flow rate is 0.5 mls/min. A series of proteins of known molecular weight and Stokes' diameter as well as human HDL are preferably used as standards to calibrate the column. The proteins and lipoprotein complexes are monitored by absorbance or scattering of light of wavelength 254 or 280 nm.

The mediators of RCT of the invention can be complexed with a variety of lipids, including saturated, unsaturated, natural and synthetic lipids and/or phospholipids. Suitable lipids include, but are not limited to, small alkyl chain phospholipids, egg phosphatidylcholine, soybean phosphatidylcholine, dipalmitoylphosphatidylcholine, dimyristoylphosphatidylcholine, distearoylphosphatidylcholine 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine dioleophosphatidylethanolamine, dilauroylphosphatidylglycerol phosphatidylcholine, phosphatidylserine, phosphatidylethanolamine, phosphatidylinositol, sphingomyelin, sphingolipids, phosphatidylglycerol, diphosphatidylglycerol, dimyristoylphosphatidylglycerol, dipalmitoylphosphatidylglycerol, distearoylphosphatidylglycerol, dioleoylphosphatidylglycerol, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, brain sphingomyelin, dipalmitoylsphingomyelin, distearoylsphingomyelin, phosphatidic acid, galactocerebroside, gangliosides, cerebrosides, dilaurylphosphatidylcholine, (1,3)-D-mannosyl-(1,3)diglyceride, aminophenylglycoside, 3-cholesteryl-6′-(glycosylthio)hexyl ether glycolipids, and cholesterol and its derivatives.

The pharmaceutical formulation of the invention contain the peptide mediators of RCT or the peptide-lipid complex as the active ingredient in a pharmaceutically acceptable carrier suitable for administration and delivery in vivo. As the peptides may contain acidic and/or basic termini and/or side chains, the peptides can be included in the formulations in either the form of free acids or bases, or in the form of pharmaceutically acceptable salts.

Injectable preparations include sterile suspensions, solutions or emulsions of the active ingredient in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives.

Alternatively, the injectable formulation may be provided in powder form for reconstitution with a suitable vehicle, including but not: limited to sterile pyrogen free water, buffer, dextrose solution, etc., before use. To this end, the mediators of RCT may be lyophilized, or the co-lyophilized peptide-lipid complex may be prepared. The stored preparations can be supplied in unit dosage forms and reconstituted prior to use in vivo.

For prolonged delivery, the active ingredient can be formulated as a depot preparation, for administration by implantation; e.g., subcutaneous, intradermal, or intramuscular injection. Thus, for example, the active ingredient may be formulated with suitable polymeric or hydrophobic materials (e.g., as an emulsion in an acceptable oil) or ion exchange resins, or as sparingly soluble derivatives; e.g., as a sparingly soluble salt form of the mediators of RCT.

Alternatively, transdermal delivery systems manufactured as an adhesive disc or patch which slowly releases the active ingredient for percutaneous absorption may be used. To this end, permeation enhancers may be used to facilitate transdermal penetration of the active ingredient. A particular benefit may be achieved by incorporating the mediators of RCT of the invention or the peptide-lipid complex into a nitroglycerin patch for use in patients with ischemic heart disease and hypercholesterolemia.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For buccal administration, the compositions may take the form of tablets or lozenges formulated in conventional manner. For rectal and vaginal routes of administration, the active ingredient may be formulated as solutions (for retention enemas) suppositories or ointments.

For administration by inhalation, the active ingredient can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The peptide mediators of RCT and/or peptide-lipid complexes of the invention may be administered by any suitable route that ensures bioavailability in the circulation. This can be achieved by parenteral routes of administration, including intravenous (IV), intramuscular (IM), intradermal, subcutaneous (SC) and intraperitoneal (IP) injections. However, other routes of administration may be used. For example, absorption through the gastrointestinal tract can be accomplished by oral routes of administration (including but not limited to ingestion, buccal and sublingual routes) provided appropriate formulations (e.g., enteric coatings) are used to avoid or minimize degradation of the active ingredient, e.g., in the harsh environments of the oral mucosa, stomach and/or small intestine. Oral administration has the advantage of easy of use and therefore enhanced compliance. Alternatively, administration via mucosal tissue such as vaginal and rectal modes of administration may be utilized to avoid or minimize degradation in the gastrointestinal tract. In yet another alternative, the formulations of the invention can be administered transcutaneously (e.g., transdermally), or by inhalation. It will be appreciated that the preferred route may vary with the condition, age and compliance of the recipient.

The actual dose of peptide mediators of RCT or peptide-lipid complex used will vary with the route of administration, and should be adjusted to achieve circulating plasma concentrations of 1.0 mg/l to 2 g/l. Data obtained in animal model systems described herein show that the ApoA-I agonists of the invention associate with the HDL component, and have a projected half-life in humans of about five days. Thus, in one embodiment, the mediators of RCT can be administered by injection at a dose between 0.5 mg/kg to 100 mg/kg once a week. In another embodiment, desirable serum levels may be maintained by continuous infusion or by intermittent infusion providing about 0.1 mg/kg/hr to 100 mg/kg/hr.

Toxicity and therapeutic efficacy of the various mediators of RCT can be determined using standard pharmaceutical procedures in cell culture or experimental animals for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀ /ED₅₀. ApoA-I peptide agonists which exhibit large therapeutic indices are preferred.

Other Uses

The mediators of RCT agonists of the invention can be used in assays in vitro to measure serum HDL, e.g., for diagnostic purposes. Because the mediators of RCT associate with the HDL and LDL component of serum, the agonists can be used as “markers” for the HDL and LDL population. Moreover, the agonists can be used as markers for the subpopulation of HDL that are effective in RCT. To this end, the agonist can be added to or mixed with a patient serum sample; after an appropriate incubation time, the HDL component can be assayed by detecting the incorporated mediators of RCT. This can be accomplished using labeled agonist (e.g., radiolabels, fluorescent labels, enzyme labels, dyes, etc.), or by immunoassays using antibodies (or antibody fragments) specific for the agonist.

Alternatively, labeled agonist can be used in imaging procedures (e.g., CAT scans, MRI scans) to visualize the circulatory system, or to monitor RCT, or to visualize accumulation of HDL at fatty streaks, atherosclerotic lesions, etc. (where the HDL should be active in cholesterol efflux).

### Assays for Analysis of Mediators of Reverse Cholesterol Transport

LCAT Activation Assay

The mediators of RCT in accordance with preferred embodiments of the present invention can be evaluated for potential clinical efficacy by various in vitro assays, for example, by their ability to activate LCAT in vitro. In the LCAT assay, substrate vesicles (small unilamellar vesicles or “SUVs”) composed of egg phophatidylcholine (EPC) or 1-palmitoyl-2-oleyl-phosphatidyl-choline (POPC) and radiolabelled cholesterol are preincubated with equivalent masses either of peptide or ApoA-I (isolated from human plasma). The reaction is initiated by addition of LCAT (purified from human plasma). Native ApoA-I, which was used as positive control, represents 100% activation activity. “Specific activity” (i.e., units of activity (LCAT activation)/unit of mass) of the molecular mediators can be calculated as the concentration of mediator that achieves maximum LCAT activation. For example, a series of concentrations of the peptide (e.g., a limiting dilution) can be assayed to determine the “specific activity” for the peptide—the concentration which achieves maximal LCAT activation (i.e., percentage conversion of cholesterol to cholesterol ester) at a specific timepoint in the assay (e.g., 1 hr.). When plotting percentage conversion of cholesterol at, e.g., 1 hr., against the concentration of peptide used, the “specific activity” can be identified as the concentration of peptide that achieves a plateau on the plotted curve.

Preparation of Substrate Vesicles

The vesicles used in the LCAT assay are SUVs composed of egg phosphatidylcholine (EPC) or 1-palmitoyl-2-oleyl-phosphatidylcholine (POPC) and cholesterol with a molar ratio of 20:1. To prepare a vesicle stock solution sufficient for 40 assays, 7.7 mg EPC (or 7.6 mg POPC; 10 μmol), 78 μg (0.2 μmol) 4-¹⁴C-cholesterol, 116 μg cholesterol (0.3 μmol) are dissolved in 5 ml xylene and lyophilized. Thereafter 4 ml of assay buffer is added to the dry powder and sonicated under nitrogen atmosphere at 4° C. Sonication conditions: Branson 250 sonicator, 10 mm tip, 6×5 minutes; Assay buffer: 10 mM Tris, 0.14 M NaCl, 1 mM EDTA, pH 7.4. The sonicated mixture is centrifuged 6 times for 5 minutes each time at 14,000 rpm (16,000×g) to remove titanium particles. The resulting clear solution is used for the enzyme assay.

Purification of LCAT

For the LCAT purification, dextran sulfate/Mg²+ treatment of human plasma is used to obtain lipoprotein deficient serum (LPDS), which is sequentially chromatographed on Phenylsepharose, Affigelblue, ConcanavalinA sepharose and anti-ApoA-I affinity chromatography.

Preparation of LPDS

To prepare LPDS, 500 ml plasma is added to 50 ml dextran sulfate (MW=500,000) solution. Stir 20 minutes. Centrifuge for 30 minutes at 3000 rpm (16,000×g) at 4° C. Use supernatant (LPDS) for further purification (ca. 500 ml).

Phenylsepharose Chromatography

The following materials and conditions were used for the phenylsepharose chromatography. Solid phase: phenylsepharose fast flow, high subst. grade, Pharmaciacolumn: XK26/40, gel bed height: 33 cm, V=ca, 175 mlflow rates: 200 ml/hr (sample)wash: 200 ml/hr (buffer)elution: 80 ml/hr (distilled water)buffer: 10 mM Tris, 140 mM NaCl, 1 mM EDTA pH 7.4, 0.01% sodium azide.

Equilibrate the column in Tris-buffer, add 29 g NaCl to 500 ml LPDS and apply to the column., Wash with several volumes of Tris buffer until the absorption at 280 nm wavelength is approximately at the baseline, then start the elution with distilled water. The fractions containing protein are pooled (pool size: 180 ml) and used for Affigelblue chromatography.

Affigelblue Chromatography

The phenylsepharose pool is dialyzed overnight at 4° C. against 20 mM Tris-HCl, pH7.4, 0.01% sodium azide. The pool volume is reduced by ultrafiltration (Amicon YM30) to 50-60 ml and loaded on an Affigelblue column. Solid phase: Affigelblue, Biorad, 153-7301 column, XK26/20, gel bed height: ca. 13 cm; column volume: approx. 70 ml. Flow rates: loading: 15 ml/h wash: 50 ml/h. Equilibrate column in Tris-buffer. Apply phenylsepharose pool to column. Start in parallel to collect fractions. Wash with Tris-buffer. The pooled fractions (170 ml) were used for ConA chromatography.

ConA Chromatography

The Affigelbtue pool was reduced via Amicon (YM30) to 30-40 ml and dialyzed against ConA starting buffer (1 mM Tris HCl pH7.4; 1 mM MgCl₂, 1 mM MnCl₂, 1 mM CaCl₂, 0.01% sodium azide) overnight at 4° C. Solid phase: ConA sepharose (Pharmacia) column: XK26/20, gel bed height: 14 cm (75 ml). Flow rates: loading 40 ml/h washing (with starting buffer): 90 ml/h elution: 50 ml/h, 0.2M Methyl-α-D-mannoside in 1 mM Tris, pH 7.4. The protein fractions of the mannoside elutions were collected (110 ml), and the volume was reduced by ultrafiltration (YM30) to 44 ml. The ConA pool was divided in 2 ml aliquots, which are stored at −20° C.

Anti-ApoA-I Affinity Chromatography

Anti-ApoA-I affinity chromatography was performed on Affigel-Hz material (Biorad), to which the anti-ApoA-I abs have been coupled covalently. Column: XK16/20, V=16 ml. The column was equilibrated with PBS pH 7.4. Two ml of the ConA pool was dialyzed for 2 hours against PBS before loading onto the column. Flow rates: loading: 15 ml/hour washing (PBS) 40 ml/hour. The pooled protein fractions (V=14 ml) are used for LCAT assays. The column is regenerated with 0.1 M. Citrate buffer (pH 4.5) to elute bound A-I (100 ml), and immediately after this procedure reequilibrated with PBS.

Pharmacokinetics of the Mediators of RCT

The following experimental protocols can be used to demonstrate that the mediators of RCT are stable in the circulation and associate with the HDL component of plasma.

Synthesis and/or Radiolabeling of Peptide Agonists

¹²⁵I-Labeled LDL was prepared by the iodine monochloride procedure to a specific activity of 500-900 cpm/ng (Goldstein and Brown 1974 J. Biol. Chem. 249:5153-5162). Binding and degradation of low density lipoproteins by cultured human fibroblasts were determined at final specific activities of 500-900 cpm/ng as described (Goldstein and Brown 1974 J. Biol. Chem. 249:5153-5162). In every case, >99% radioactivity was precipitable by incubation of the lipoproteins at 4° C. with 10% (wt/vol) trichloroacetic acid (TCA). The Tyr residue was attached to N-Terminus of each peptide to enable its radioiodination. The peptides were radioiodinated with Na¹²⁵I (ICN), using lodo-Beads (Pierce Chemicals) and following the manufacturer's protocol, to a specific activity of 800-1000 cpm/ng. After dialysis, the precipitable radioactivity (10% TCA) of the peptides was always >97%.

Alternatively, radiolabeled peptides could be synthesized by coupling ¹⁴C-labeled Fmoc-Pro as the N-terminal amino acid. L-[U-¹⁴ C]X, specific activity 9.25 GBq/mmol, can be used for the synthesis of labeled agonists containing X. The synthesis may be carried out according to Lapatsanis, Synthesis, 1983, 671-173. Briefly, 250 μM (29.6 mg) of unlabeled L-X is dissolved in 225 μl of a 9% Na₂ CO₃ solution and added to a solution (9% Na₂CO₃) of 9.25 MBq (250 lM) ¹⁴C-labeled L-X. The liquid is cooled down to 0° C., mixed with 600 μM (202 mg) 9-fluorenylmethyl-N-succinimidylcarbonate (Fmoc-OSu) in 0.75 ml DMF and shaken at room temperature for 4 hr. Thereafter, the mixture is extracted with Diethylether (2×5 ml) and chloroform (1×5 ml), the remaining aqueous phase is acidified with 30% HCl and extracted with chloroform (5×8 ml). The organic phase is dried over Na₂ SO₄₁ filtered off and the volume is reduced under nitrogen flow to 5 ml. The purity was estimated by TLC (CHCl₃ :MeOH:Hac, 9:1:0.1 v/v/v, stationary phase HPTLC silicagel 60, Merck, Germany) with UV detection, e.g., radiochemical purity:Linear Analyzer, Berthold, Germany; reaction yields may be approximately 90% (as determined by LSC).

The chloroform solution containing ¹⁴C-peptide X is used directly for peptide synthesis. A peptide resin containing amino acids 2-22, can be synthesized automatically as described above and used for the synthesis. The sequence of the peptide is determined by Edman degradation. The coupling is performed as previously described except that HATU (O-(7-azabenzotriazol-1-yl)1-, 1,3,3-tetramethyluroniumhexafluorophosphate) is preferably used instead of TBTU. A second coupling with unlabeled Fmoc-L-X is carried out manually.

Pharmacokinetics in Mice

In each experiment, 300-500 μg/kg (0.3-0.5 mg/kg) [or more such as 2.5 mg/k] radiolabeled peptide may be injected intraperitoneally into mice which were fed normal mouse chow or the atherogenic Thomas-Harcroft modified diet (resulting in severely elevated VLDL and IDL cholesterol). Blood samples are taken at multiple time intervals for assessment of radioactivity in plasma.

Stability in Human Serum

100 μg of labeled peptide may be mixed with 2 ml of fresh human plasma (at 37° C.) and delipidated either immediately (control sample) or after 8 days of incubation at 37° C. (test sample). Delipidation is carried out by extracting the lipids with an equal volume of 2:1 (v/v) chloroform:methanol. The samples are loaded onto a reverse-phase C₁₈ HPLC column and eluted with a linear gradient (25-58% over 33 min) of acetonitrile (containing 0.1% w TFA). Elution profiles are followed by absorbance (220 nm) and radioactivity.

Formation of Pre-β Like Particles

Human HDL may be isolated by KBr density ultra centrifugation at density d =1.21 g/ml to obtain top fraction followed by Superose 6 gel filtration chromatography to separate HDL from other lipoproteins. Isolated HDL is adjusted to a final concentration of 1.0 mg/ml with physiological saline based on protein content determined by Bradford protein assay. An aliquot of 300 μl is removed from the isolated HDL preparation and incubated with 100 μl labeled peptide (0.2-1.0 μg/μl) for two hours at 37° C. Multiple separate incubations are analyzed including a blank containing 100 μl physiological saline and four dilutions of labeled peptide. For example: (i) 0.20 μg/μl peptide:HDL ratio=1:15; (ii) 0.30 μg/μl peptide:HDL ratio=1:10; (iii) 0.60 μg/μl peptide:HDL ratio=1:5; and (iv) 1.00 μg/μl peptide:HDL ratio=1:3. Following the two hour incubation, a 200 μl aliquot of the sample (total volume=400 μl) is loaded onto a Superose 6 gel filtration colunm for lipoprotein separation and analysis and 100 μl is used to determine total radioactivity loaded.

Association of Mediators with Human Lipoproteins

The association of peptide mediators with human lipoprotein fractions can be determined by incubating labeled peptide with each lipoprotein class (HDL, LDL and VLDL) and a mixture of the different lipoprotein classes. HDL, LDL and VLDL are isolated by KBr density gradient ultracentrifugation at d=1.21 g/ml and purified by FPLC on a Superose 6B column size exclusion column (chromatography is carried out with a flow rate of 0.7 ml/min and a running buffer of 1 mM Tris (pH 8), 115 mM NaCl, 2 mM EDTA and 0.0% NaN₃). Labeled peptide is incubated with HDL, LDL and VLDL at a peptide:phospholipid ratio of 1:5 (mass ratio) for 2 h at 37° C. The required amount of lipoprotein (volumes based on amount needed to yield 1000 μg) is mixed with 0.2 ml of peptide stock solution (1 mg/ml) and the solution is brought up to 2.2 ml using 0.9% of NaCl.

After incubating for 2 hr at 37° C., an aliquot (0.1 ml) is removed for determination of the total radioactivity (e.g., by liquid scintilation counting or gamma counting depending on labeling isotope), the density of the remaining incubation mixture is adjusted to 1.21 g/ml with KBr, and the samples centrifuged at 100,000 rpm (300,000 g) for 24 hours at 4° C. in a TLA 100.3 rotor using a Beckman tabletop ultracentrifuge. The resulting supernatant is fractionated by removing 0.3 ml aliquots from the top of each sample for a total of 5 fractions, and 0.05 ml of each fraction is used for counting. The top two fractions contain the floating lipoproteins, the other fractions (3-5) correspond to proteins/peptides in solution.

Selective Binding to HDL Lipids

Human plasma (2 ml) is incubated with 20, 40, 60, 80, and 100 μg of labeled peptide peptide for 2 hr at 37° C. The lipoproteins are separated by adjusting the density to 1.21 g/ml and centrifugation in TLA 100.3 rotor at 100,000 rpm (300,000 g) for 36 hr at 4° C. The top 900 μl (in 300 μl fractions) is taken for the analysis. 50 μl from each 300 μl fraction is counted for radioactivity and 200 μl from each fraction is analyzed by FPLC (Superose 6/Superose 12 combination column).

Use of the Mediators of Reverse Cholesterol Transport in Animal Model Systems

The efficacy of the mediators of RCT of the invention can be demonstrated in rabbits or other suitable animal models.

Preparation of the Phospholipid/Peptide Complexes

Small discoidal particles consisting of phospholipid (DPPC) and peptide are prepared following the cholate dialysis method. The phospholipid is dissolved in chloroform and dried under a stream of nitrogen. The peptide is dissolved in buffer (saline) at a concentration of 1-2 mg/ml. The lipid film is redissolved in buffer containing cholate (43° C.) and the peptide solution is added at a 3:1 phospholipid/peptide weight ratio. The mixture is incubated overnight at 43° C. and dialyzed at 43° C. (24 hr), room temperature (24 hr) and 4° C. (24 hr), with three changes of buffer (large volumes) at temperature point. The complexes may be filter sterilized (0.22 μm) for injection and storage at 4° C.

Isolation and Characterization of the Peptide/Phospholipid Particles

The particles may be separated on a gel filtration column (Superose 6 HR). The position of the peak containing the particles is identified by measuring the phospholipid concentration in each fraction. From the elution volume, the Stokes radius can be determined. The concentration of peptide in the complex is determined by measuring the phenylalanine content (by HPLC) following a 16 hr acid hydrolysis.

Injection in the Rabbit

Male New Zealand White rabbits (2.5-3 kg) are injected intravenously with a dose of phospholipid/peptide complex (5 or 10 mg/kg bodyweight, expressed as peptide) in a single bolus injection not exceeding 10-15 ml. The animals are slightly sedated before the manipulations. Blood samples (collected on EDTA) are taken before and 5, 15, 30, 60, 240 and 1440 minutes after injection. The hematocrit (Hct) is determined for each sample. Samples are aliquoted and stored at −20° C. before analysis.

Analysis of the Rabbit Sera

The total plasma cholesterol, plasma triglycerides and plasma phospholipids are determined enzymatically using commercially available assays, for example, according to the manufacturer's protocols (Boehringer Mannheim, Mannheim, Germany and Biomerieux, 69280, Marcy-L'etoile, France).

The plasma lipoprotein profiles of the fractions obtained after the separation of the plasma into its lipoprotein fractions may be determined by spinning in a sucrose density gradient. For example, fractions are collected and the levels of phospholipid and cholesterol can be measured by conventional enzymatic analysis in the fractions corresponding to the VLDL, ILDL, LDL and HDL lipoprotein densities.

### WORKING EXAMPLES

The short-term goal was to identify compound mimics of ApoA-I that function in HDL-mediated cholesterol transport to the liver. The long term goal was to modify the compounds so they can interact with a subset of lipoproteins and target them to the liver and amplify the rate of cholesterol-rich lipoproteins catabolism (reverse cholesterol transport). Unlike the current treatments (resins, statins, fibrates), that regulate cholesterol transport to peripheral tissues, the approach adopted herein involves amplification of RCT rate by increasing of the HDL cholesterol (HDL-C) levels and catabolism of the cholesterol-rich low density lipoproteins. The rational for this approach is the long acknowledged inverse relationship between rate of RCT and cardiovascular risk.

Lipoprotein isolation—Human plasma lipoproteins were isolated from fresh fasting plasma that was obtain by plasmapheresis from normal donors. LDL (d=1.019-1.063 g/ml) was isolated under strict sterile, endotoxin-free conditions by sequential ultracentrifugation using KBr for density adjustment. It was dialyzed against 0.15 mM NaCl containing 0.3 mM EDTA and probucol, pH 7.4, filter-sterilized, and stored at 4° C.

Radioiodination—¹²⁵I-Labeled LDL was prepared by the iodine monochloride procedure at final specific activities of about 500-900 cpm/ng (Goldstein and Brown 1974 J. Biol. Chem. 249:5153-5162). Binding and degradation of low density lipoproteins by cultured human fibroblasts were performed at final specific activities of about 500-900 cpm/ng (Goldstein and Brown 1974 J. Biol. Chem. 249:5153-5162). In every case, >99% radioactivity was precipitable by incubation of the lipoproteins at 4° C. with 10% (wt/vol) trichloroacetic acid (TCA). The Tyr residue was attached to N-Terminus of each peptide to enable its radioiodination. The peptides were radioiodinated with Na¹²⁵I (ICN), using lodo-Beads (Pierce Chemicals) and following the manufacturer's protocol, to a specific activity of 800-1000 cpm/ng. After dialysis, the precipitable radioactivity (10% TCA) of the peptides was always >97%.

Plasma stability and lipoproteins distribution of peptides—The LDLR−/− mice used for these studies were males, 2 months old, and were Chow-fed. Blood was drawn from nonfasted mice into heparin-coated tubes, and was subjected to low speed centrifugation at 4° C. to obtain the plasma. To measure the plasma stability, 5-6 μg of radioiodinated peptide was added to 0.25 ml of plasma. After incubation at 37° C., aliquots were removed and subjected to precipitation with 10% TCA. To study the association of ApoA-I peptides with lipoproteins and/or other proteins in plasma, 6-8 μg of radioiodinated peptide was incubated for 2 hrs at 37° C. with 0.12 ml of mouse plasma. Following incubation, the mixture was separated on 1% agarose gel (Paragon system, Beckman Coulter) according to manufacturer's instructions, and radioactivity was quantified in the bands representing LDL, HDL, and albumin.

Tissue distribution of peptides −12 μg of radioiodinated peptide was injected intravenously into the tail vein of apo A-1 deficient mice under metofane anesthesia. The mice were sacrificed 40 min after injection, and blood was removed by perfusion with cold PBS via a cannula into the left ventricle. 40 min later mice were bled by retroorbital bleeding into heparinized tubes.

Mediator-LDL complexes—Peptide/lipoprotein complexes were formed by incubation of excess amounts of radiolabeled peptide (SEQ ID NO: 1) for 2 hrs at 25° C. with human plasma LDL diluted into PBS at a molar ratio 25:1. The complexes were extensively dialyzed at 4° C. to remove free peptide against PBS containing 20 μM of butylated hydroxy toluene (BHT) until counted in dialyzing solution radioactivity was less than 400-600 cpm/ml for at least 2 hrs. Formed complexes were used immediately.

Plasma clearance and tissue distribution of SEQ ID NO: 1-LDL complexes—¹²⁵I- LDL (0.2 nmol) was incubated at 37° C. in PBS alone, or with 4 nmol of SEQ ID NO: 1. After 2 h, the mixture was dialyzed against PBS containing 20 mM BHT. ¹²⁵I-LDL alone or SEQ ID NO: 1/¹²⁵I-LDL complex were injected intravenously into the tail vein of nonfasted mice under metofane anesthesia. Mice were bled at the certain time points after the injection by retroorbital bleeding into heparinized tubes. The blood was subjected to low speed centrifugation (1800 g, 4° C.) and the 10% TCA precipitable radioactivity of the plasma was measured. The mice were sacrificed 40 min after injection, and blood and nonspecifically bound radioactivity were removed by perfusion with cold PBS via a cannula into the left ventricle. An incision was made in the interior vena cava to clear the perfusate. Within 15 min, the liver, kidneys, spleen, and heart were removed, cleaned, weighed, and counted for ¹²⁵I radioactivity. Entire organs were counted with exception of liver, which was counted in pieces. Radioactivity detected per organ or per 1 g of wet tissue was expressed as a percent of the initial total injected radioactivity that was TCA precipitable.

Effect ofsingle bolus injections ofpeptides on plasma lipoprotein profile—To monitor effect of peptides on plasma cholesterol levels and its distribution among the different lipoprotein classes, the nonradioiodinated free peptides (100 μg in 100 μl of PBS) were injected intravenously into the tail vein of nonfasted C57BL/6J wild type mice, which have been placed on high fat cholate-containing diet four days prior to experiment. To control for the effect of external and/or internal nonspecific factors (stress of handling, anesthetic, blood drawing) on plasma lipoprotein profile, a similar group of mice was injected with only PBS. Different groups of mice were sacrificed before and at various times after injections and blood was drawn by retroorbital puncture and subjected to low speed centrifugation at 4° C. to obtain the plasma. Plasma samples were obtained, combined within each group (4 mice), and subjected to gel filtration chromatography on a Superose 6 (HR 10/30 column, FPLC) to monitor cholesterol lipoproteins distribution and agarose gel electrophoresis (Paragon Systems) to monitor phospholipid lipoproteins distribution.

Effect of time-released peptides on plasma lipoprotein profile—To determine relatively long-term effect of SEQ ID NO: 1 and its derivatives on plasma lipoprotein profile, the Alzet Mini-Osmotic pumps (220 ul), containing various peptides or PBS, were surgically inserted in canulated Chow fed C57BL/6J mice. The pumps with flow rate equal to 8 μl/hr were used for 20 hrs continuous infusion of peptides. The pumps with flow rate equal to 1 μl/hr were used for 160 hrs continuous infusions of peptides. At the end point (20 or 160 hrs after pumps insertion) mice were sacrificed, and blood was drawn by retroorbital puncture and subjected to low speed centrifugation at 4° C. to obtain the plasma. Bile was immediately removed from gall bladders using insulin syringes, and stored on ice until use.

Sample analysis—Total serum cholesterol and HDL cholesterol were determined using the Infinity colorimetric-enzymatic method (Sigma 401-25P) according to manufacturer's instructions, changing only the suggested 37° C. incubation time from 10 to 15 min. To determine HDL cholesterol, low density lipoproteins were precipitated from plasma using a modified Burstein-Samaille method according to manufacturer's instructions (Boehringer Mannheim 543004), modifying only the ratio of reagent:water from 4:1 to 4:2. Gall bladder derived bile was diluted twice with deionized water, and cholesterol was determined using Infinity reagents. 3α-Hydroxybile acids were quantified using a colorimetric-enzymatic method (Sigma 450).

To monitor cholesterol distribution among the different lipoprotein classes, plasma samples were combined within each group (usually 4-6 mice per group), and subjected to FPLC size-exclusion chromatography on a Superose 6 (HR 10/30 column, Amersham-Pharmacia) using an isocratic 10 mM Tris/150 mM NaCl/1 mM EDTA buffer system at a flow rate of 0.15 ml/min. Fractions (0.15 ml) were collected into 96-well plates containing 0.055 ml of 1:1 mixture of 0.5% Triton X-100 and 20 mM Sodium Cholate. Total and free cholesterol in plasma fractions was determined by using the fluorescent method of W. Gamble, et al. (Gamble et al., 1978, Journal Lipid Res., 16:1068-1070). 96-well plates were read on Dual-Scanning Microplate Spectrofluorometer Gemini XS (Molecular Devices). The areas under the lipoprotein peaks were quantified by using Unicorn software (Version 3.21.02). The amount of esterified cholesterol was assessed by subtraction of free cholesterol from total cholesterol.

To monitor the distribution of phospholipid among the lipoprotein classes, plasma samples were combined within each group (usually 4-6 mice per group), and subjected to agarose gel electrophoresis, which was carried out using the Paragon system (Beckman Coulter) according to manufacturer's instructions, with following staining with Paragon Lipo Stain (Beckman Coulter 655910). Once dried, the gels were scanned on Personal Densitometer SI (Molecular Dynamics) and bands were quantified using ImageQuant™ software (Verssion 5.2).

Effect of peptides on PLTP activity—PLTP activity was measured using a fluorescent kit (Cardiovascular Targets, Inc., P7700). PLTP source was serum obtained from C57BL/6J male 2 months old mice, which were Chow-fed or maintained on high fat cholate containing diet for four days. 1× Mouse serum was preincubated with PBS or 0.4, 2, 5, and 10 μg of peptides at RT for 30 minutes. Following preincubation, the mixture was diluted 10 times, and 10 μl (0.8 υl nest serum) was immediately mixed into reaction wells of pre-chilled 96-well plate, containing assay system (Cardiovascular Targets, Inc., P7700). The microplates were read at 37° C. in SpectraMax 190 (Molecular Devices), for 30 minutes.

LDL mediated cholesterol accumulation in human HepG2 cells-HepG2 cells were cultured at 37° C. in DMEM supplemented with 10% FBS. 24 hrs before the experiment cells were plated in a 24 wells plate at the density of 2.5×10⁵ per well in serum free media (500 μl RPMI supplemented with 1% Nutridoma-HU, Roche, 903454321) to permit up-regulation of LDL-receptors. On the day of experiment, cells were washed twice with PBS, and 25 μg of isolated human LDL (Academy Bio-Medical Co., 20P-L101) preincubated with PBS or peptides for 1 hr at RT was added to the cells in 500 μl of SFM with following incubation at 37° C. for 6 hrs. Following incubation, media was removed, cells were washed twice with RT PBS, and total cholesterol was extracted by Hexane-Isopropanol mixture (3:2), and dried under the Nitrogen gas. The dried samples were solubilized in 160 μl TE buffer (10 mM Tris-HCL, 150 mM NaCl, 1 mM EDTA) containing 0.1% Triton X-100 and 4 mM of Sodium Cholate. Total and free cholesterol in the samples were quantified using the fluorescent method of W. Gamble, et al. (Gamble et al., 1978, Journal Lipid Res., 16:1068-1070). The amount of esterified cholesterol was assessed by subtraction of free cholesterol from total cholesterol. The results are shown in FIG. 24.

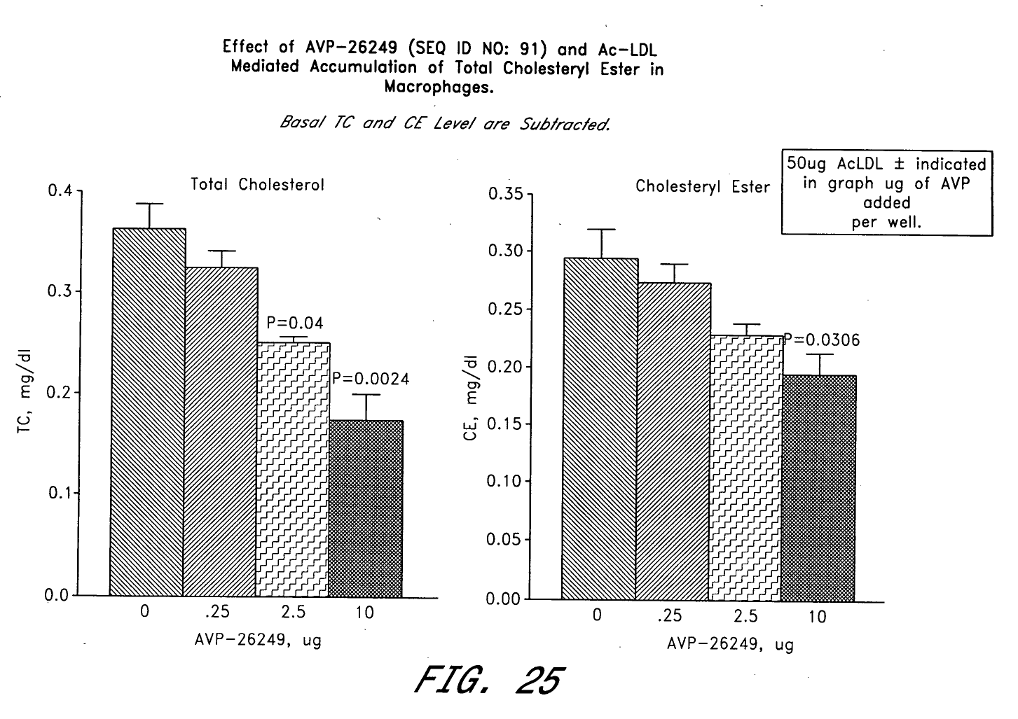

Ac-LDL mediated cholesterol accumulation in human macrophages—THP-1 cells were cultured at 37° C. in RPMI supplemented with 10% FBS. 48 hrs before the loading experiment cells were plated in a 24 wells plate at the density of 1×10⁶ per well in serum free media (500 ul RPMI supplemented with 1% Nutridoma-HU, Roche, Lot#: 903454321) in presence of 5×10⁻⁸ M PMA. On the day of experiment, PBS or 50 ug of human acetylated LDL, (Biomedical technologies Inc. BT-906) pre-incubated with PBS or peptides for 1 hr at room temperature were added to the cells. Treated cells were incubated for 24 hr at 37° C. in a humidified 5% CO2 incubator. Following incubation, media was removed, cells were washed 2× with 37° C. PBS and Hexane-Isopropanol (3:2) mixture was added to the cells to extract cholesterol. 30 min later samples were transfered into glass tubes and dried under the Nitrogen. Formed pellet was solubilized in 160 ul of TE buffer (10 mM Tris-HCl, 150 mM NaCl, 1 mM EDTA) containing 0.1% Triton X-100 & 4 mM Sodium Cholate. Total and free cholesterol were quantified by using the fluorescent method of W. Gamble, et al (Gamble et al, 1978, Journal Lipid Res., 16:1068-1070). The amount of esterified cholesterol was assessed by subtraction of free cholesterol from total cholesterol. The results are shown in FIG. 25.

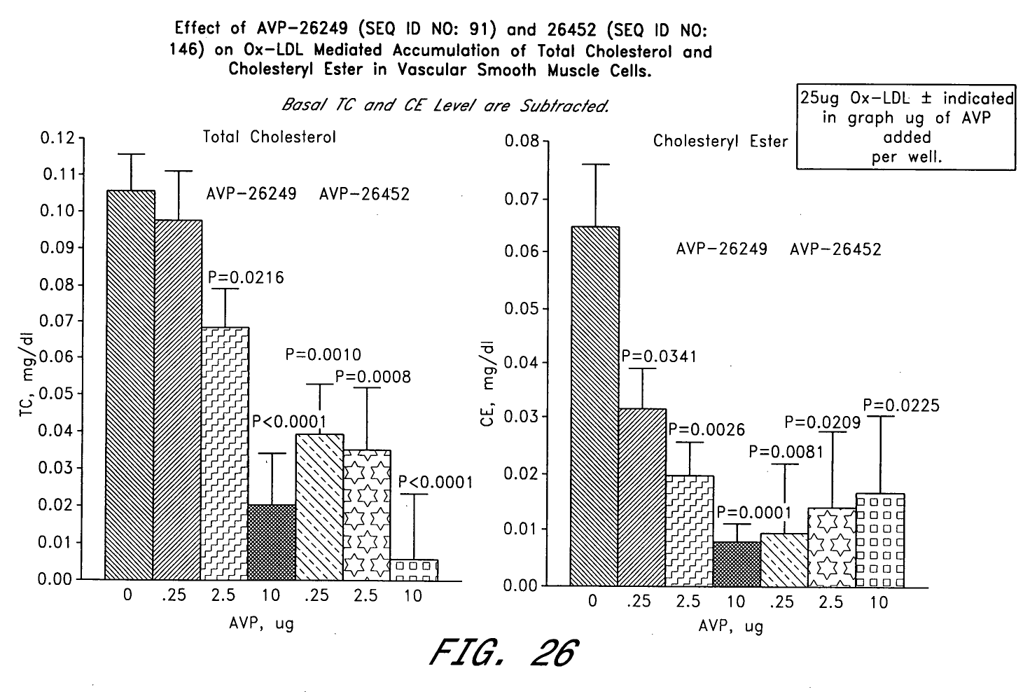

Oxidized-LDL mediated cholesterol accumulation in human vascular smooth muscle cells—vascular smooth muscle cells were cultured at 37° C. In SmGm-2 (Cambrex, cc-3182) supplemented with 5% FBS. 24 hrs before the experiment cells were plated in a 24 wells plate at the density of 85,000 per well in 500 ul of serum free assay media (SmGM-2 supplemented with 1% Nutridoma-HU, Roche, Lot#: 903454321). On the day of experiment, cells were washed twice with PBS, and 25 ug of Oxidized human LDL (Biomedical technologies Inc. BT-906) pre-incubated with PBS or peptides for 1 hr at room temperature were added to the cells in 500 ul of serum free assay media (SFM). Treated cells were incubated for 24 hr at 37° C. in a humidified 5% CO2 incubator. Following the incubation, media was removed, cells were washed 2× with 37° C. PBS and cholesterol was extracted by hexane-isopropanol (3:2) mixture. Samples were dried under the nitrogen. Dried samples were solubilized in 160 ul of TE buffer (10 mM Tris-HCl, 150 mM NaCl, 1 mM EDTA) containing 0.1% Triton X-100 and 4 mM Sodium Cholate. Total and free cholesterol in the samples were quantified using the fluorescent method described in W. Gamble, et al (Gamble et al, 1978, Journal Lipid Res., 16:1068-1070). The amount of esterified cholesterol was assessed by subtraction of free cholesterol from total cholesterol. The results are shown in FIG. 26.

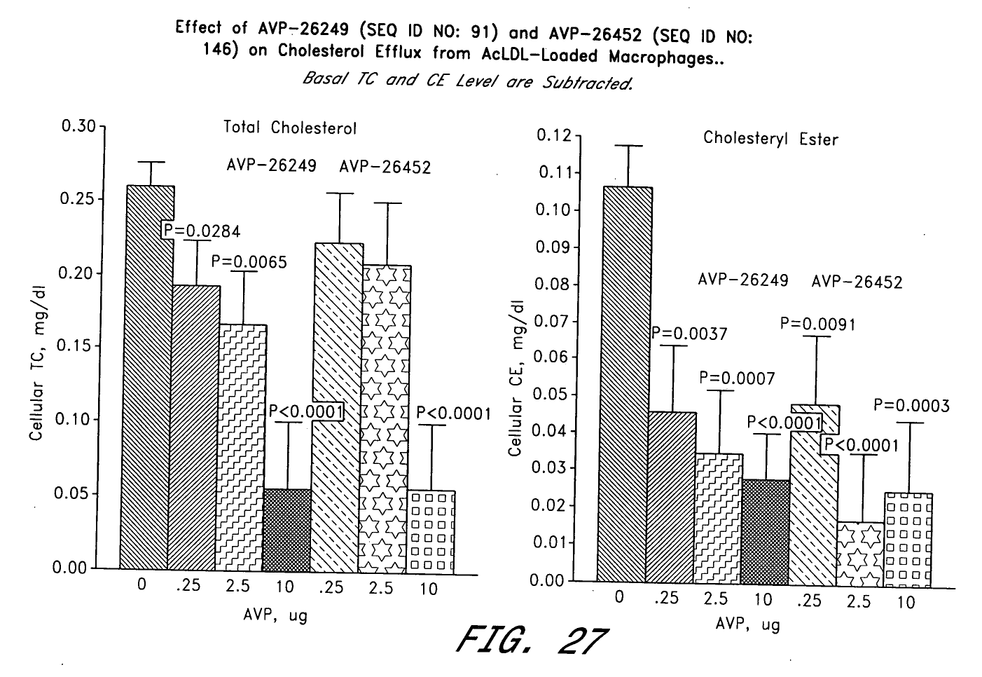

Cholesterol efflux from Ac-LDL preloaded human macrophages—THP-1 cells were cultured at 37° C. in RPMI supplemented with 10% FBS. 48 hrs before the loading experiment cells were plated in a 24 wells plate at the density of 1×10⁶ per well in 500 ul of serum free media (RPMI supplemented with 1% Nutridoma-HU, Roche, Lot#: 903454321) ) in presence of 5×10⁻⁸ M PMA. On the day of experiment, PBS or 50 ug of human acetylated LDL, (Biomedical technologies Inc. BT-906) were added to the cells in serum free media in presence of 5×10⁻⁸M PMA. Treated cells were incubated for 24 hr at 37° C. in a humidified 5% CO2 incubator. Following incubation, media was removed, cells were washed with serum free media, and PBS or compounds were added to the cells in 500 ul of serum free media (no PMA). Treated cells were incubated at 37° C. in a humidified 5% C02 incubator for another 48 hr. Compounds were refreshed every 24 hr. Following incubation, media was removed, cells were washed 2× with 37° C. PBS, and Hexane-Isopropanol (3:2) mixture was added to the cells to extract cholesterol. 30 min later samples were transfered into glass tubes and dried under the Nitrogen. Formed pellet was solubilized in 160 ul of TE buffer (10 mM Tris-HCl, 150 mM NaCl, 1 mM EDTA) containing 0.1% Triton X-100 & 4 mM Sodium Cholate. Total and free cholesterol were quantified by using the fluorescent method of W. Gamble, et al (Gamble et al, 1978, Journal Lipid Res., 16:1068-1070). The amount of esterified cholesterol was assessed by subtraction of free cholesterol from total cholesterol. The results are shown in FIG. 27.

We analyzed the ApoA-I primary, secondary, and tertiary structure to design the series of compounds. The potential lead compounds, to affect the lipoprotein metabolism, preferably exhibit both ability to bind to lipoproteins and to the liver lipoprotein binding sites. Therefore, all the compounds were radiolabeled and screened for their tissue distribution in vivo (mice) and ability to bind mouse plasma lipoproteins in vitro.

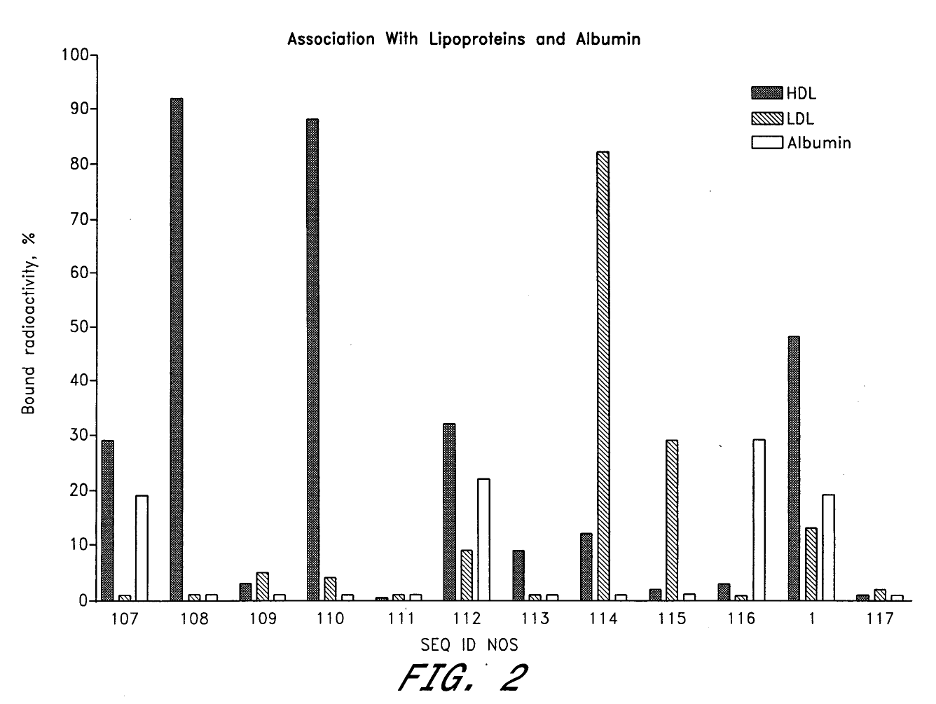

Characterization of the peptide mediators of RCT in vitro—Twelve compounds were radioiodinated and incubated with plasma from mice deficient in the LDL-receptor (LDLR−/−). These mice have a lipoprotein profile similar to that in humans. Following incubation at 37° C. for 2 hrs, the mixture was separated on agarose gels and radioactivity was quantified in the bands representing LDL, HDL, and albumin. Results are summarized in FIG. 2. Based on these results, the compounds that demonstrated significant association with lipoproteins were further characterized in vivo.

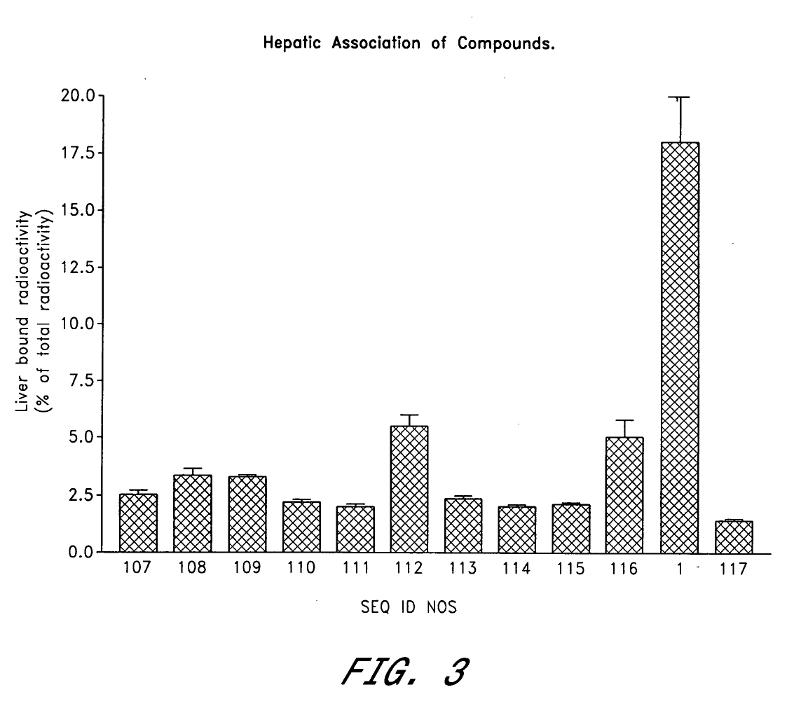

Characterization of the peptide mediators of RCT in vivo—The radiolabeled compounds were injected intravenously into mice. At the end point mice were bled and perfused extensively to remove circulating (nonspecific) radioactivity. Whole blood and potential target organs were harvested and counted. The total radioactivity in the collected organs and the entire blood volume of the mouse was calculated and the organ-bound radioactivity was expressed as a percentage of the total radioactivity. The results for organs bound radioactivity following injections of compounds are shown in FIG. 3. Based on these data, SEQ ID NO: 1 was selected for further in vivo characterization.

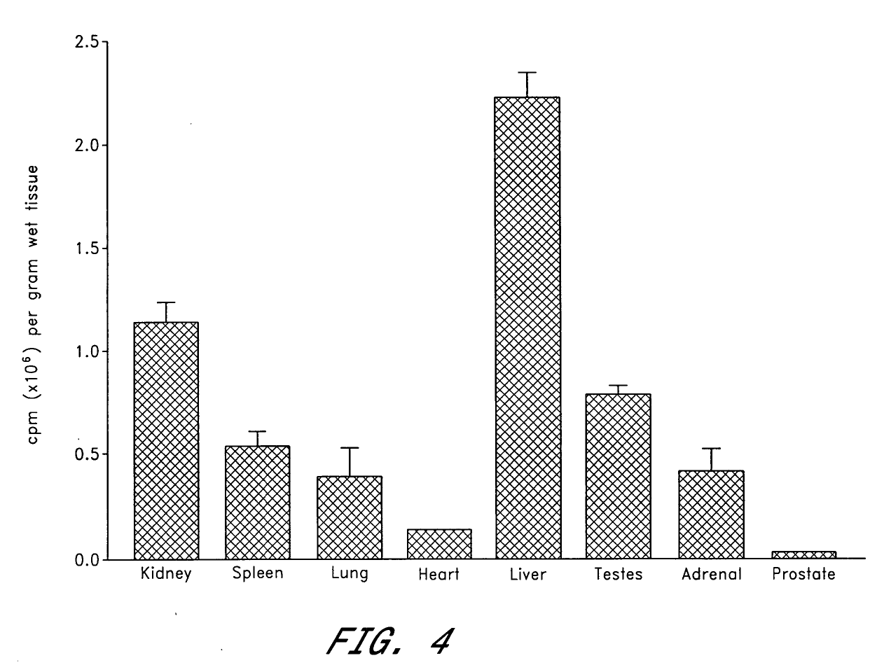

Further characterization of SEQ ID NO: 1 in vivo—FIG. 4 illustrates the organ distribution of SEQ ID NO: 1. Because this compound associated with the liver preferentially to a dramatic extent, compare to other organs and peptides, we chose to focus our efforts on this compound.

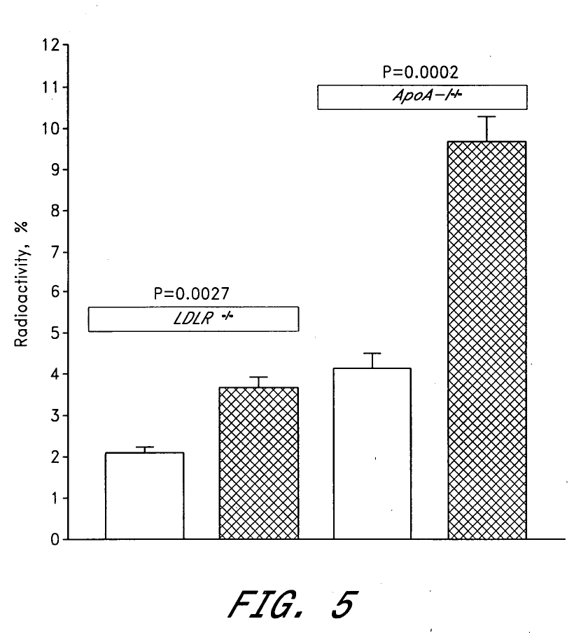

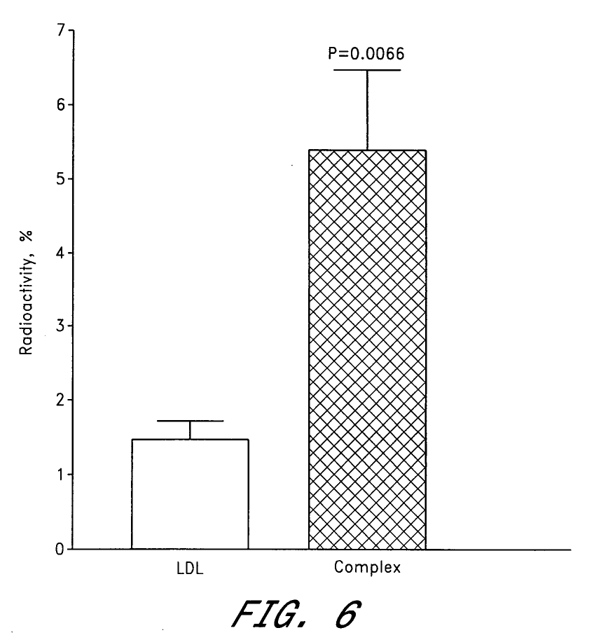

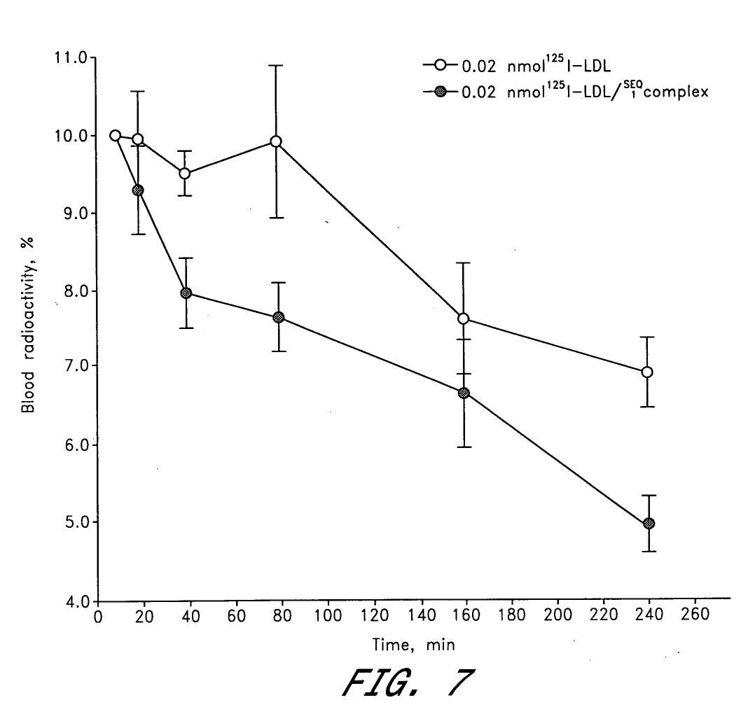

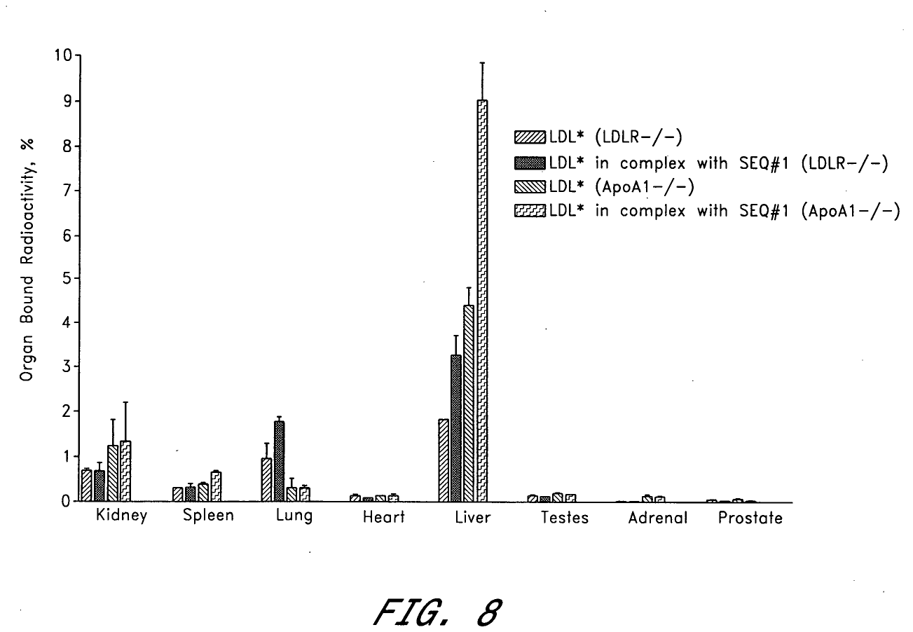

To determine if SEQ ID NO: 1 can associate with human LDL, we attempted to form the complex of ¹²⁵I-SEQ ID NO: 1 with isolated human LDL. It has been demonstrated that SEQ ID NO: 1 forms the stable [LDL-¹²⁵I-SEQ ID NO: 1] complex, where approximately 6 to 8 copies of [¹²⁵I-SEQ ID NO: 1] were bound per LDL particle. To determine if SEQ ID NO: 1 influences lipoprotein transport to the liver, we injected the 1251I-LDL alone or in complex with SEQ ID NO: 1 into LDLR−/− mice (no LDL-receptor) and A-I−/− mice, which have a functional hepatic LDL-receptor. Liver associated radioactivity was assessed and results are presented in FIG. 5. In both mouse genotypes, complexing of [¹²⁵I-LDL] with SEQ ID NO: 1 before injection resulted in a significant increase in liver-bound [¹²⁵I-LDL]. These data indicate that enhanced liver binding of [¹²⁵I-LDL-SEQ ID NO: 1] complex (compare to [¹²⁵I-LDL]) is mediated by: (a) unknown-yet liver lipoprotein binding sites; (b) LDL-receptors. To assess the contribution of LDL-receptors alone, the binding of [¹²⁵I-LDL] and [¹²⁵I-LDL-SEQ ID NO: 1] to the liver of LDLR−/− mice was subtracted from their respective binding to the liver of A-I−/− mice, and data were normalized per gram of wet tissue. The result of this subtraction is presented in FIG. 6 and shows substantial increase in complex binding to the LDL-receptors compare to [¹²⁵I-LDL] alone. These data are in a good agreement with enhanced plasma clearance of ¹²⁵I-LDL-SEQ ID NO: 1 complex compare to ¹²⁵I-LDL alone when injected in ApoAl−/− mice (FIG. 7). Influence of SEQ ID NO: 1 on ¹²⁵I-LDL binding to kidney, spleen, lung, heart, testis, adrenal, and prostate is shown in FIG. 8. It is noteworthy that no increase in ¹²⁵I-LDL-SEQ ID NO: 1 complex binding to the heart was observed.

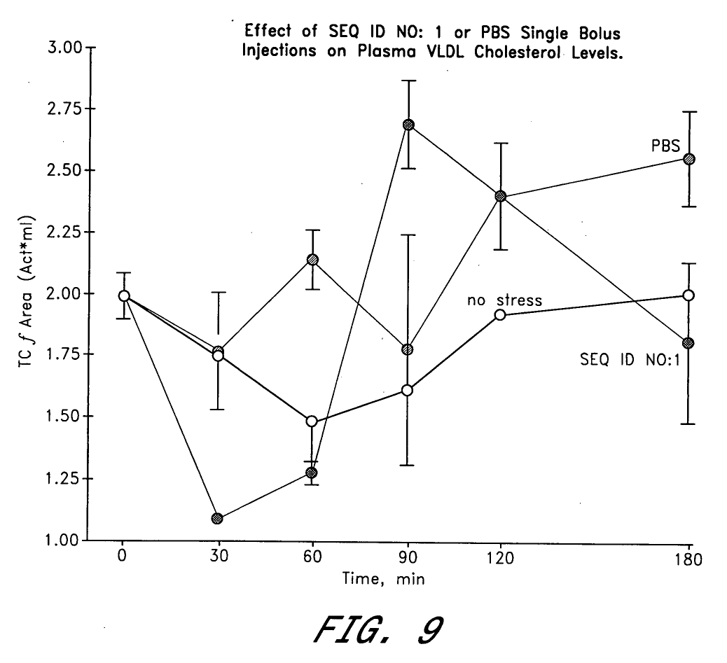

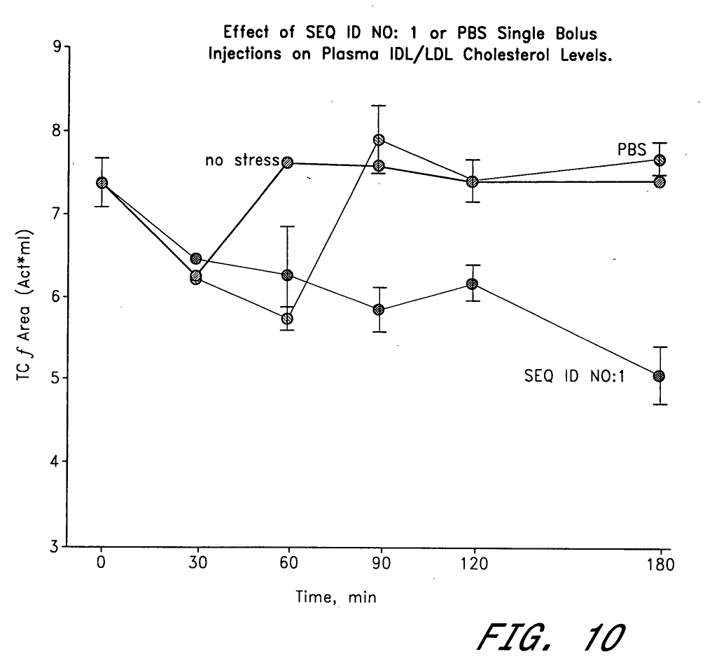

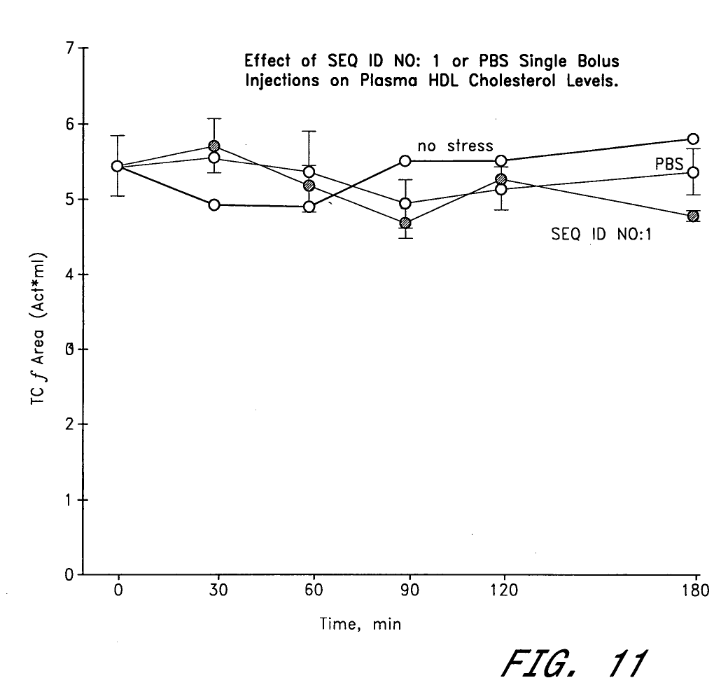

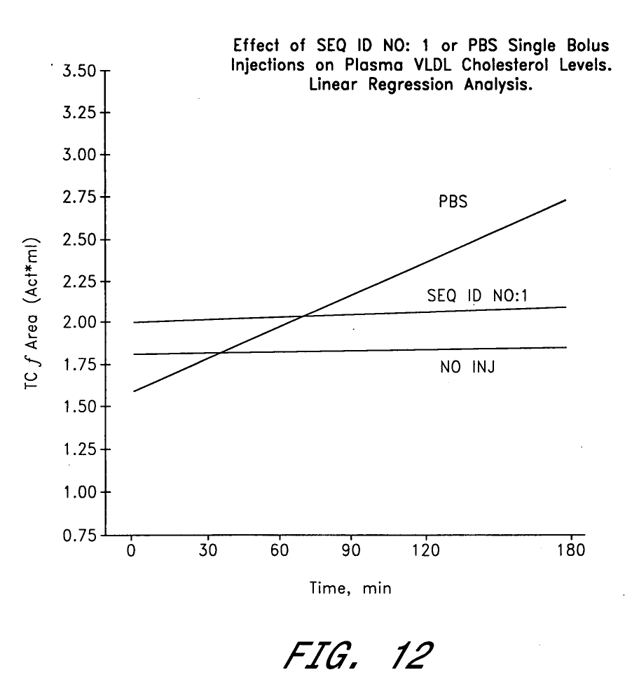

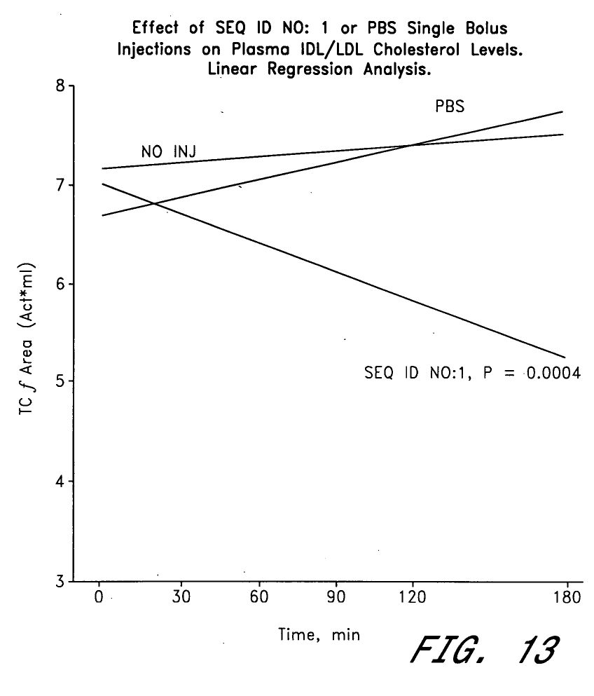

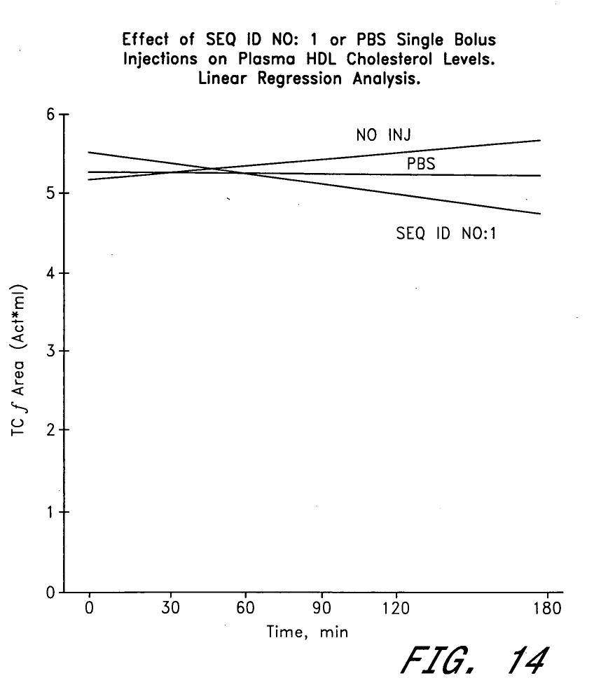

Impact of SEQ ID NO: 1 on lipoprotein metabolism (Single bolus injections)—We examined the impact of SEQ ID NO: 1 on cholesterol metabolism in mildly hyperlipidemic wild type C57BL/6J mice fed a cholate-containing high fat diet (HFC). Mice were divided into two groups—control and experimental. Each group contained 4 mice. Experimental mice were injected intravenously with SEQ ID NO: 1 (100 μg in 100 μl of PBS), whereas control group—with 100 ul of PBS. Different groups of mice were bled at 0 min (no injections), 30, 60, 90, 120, and 180 min after injections, plasma was obtained, combined within each group, and subjected FPLC analysis. Cholesterol distribution among the different lipoprotein classes was assessed and data were expressed as cholesterol content (Act*ml) or area under the VLDL, IDL/LDL, and HDL peaks, respectively. The results are presented in FIGS. 9, 10 and 11. Although, there was no significant impact of SEQ ID NO: 1 on VLDL level over the entire time course, kinetics of the liver response was noticeably different compare to PBS control (FIG. 9). Significant lowering of IDL/LDL (proatherogenic and atherogenic lipoproteins) was observed at 90, 120, and 180 min after SEQ ID NO: 1 injections (FIG. 10). Effect of SEQ ID NO: 1 lasts up to 180 min and completely vanished at 240 min time point. These data are in agreement with in vivo half-life data of ¹²⁵I-SEQ ID NO: 1, which is approximately 2 to 3 hrs. There was observed slight but significant decrease of HDL-C levels after single bolus injections of SEQ ID NO: 1 (FIG. 11), indicating possible increase of HDL clearance. Linear regression analysis of single bolus injections data is presented in FIGS. 12-14 and shows significant impact of SEQ ID NO: 1 on plasma levels of pro- and atherogenic lipoproteins (IDL and LDL).

Effect of “frame-shift” derived peptides on lipoprotein metabolism (Single bolus injections)—We performed the functional frame-shift of the entire region of mouse apolipoprotein A1 where SEQ ID NO: 1 derived from (helix 6, Pro166- Pro188, precursor protein). Frame-shift was done in both directions (form N- to C- and from C- to N-Terminus). Sixteen (16) peptides were designed, synthesized and tested for possible effects on cholesterol metabolism of mildly hyperlipidemic C57BL/6J mice fed a cholate-containing high fat diet (HFC). Mice were divided into two groups—control and experimental. Each group contained 4 mice. Experimental mice were injected intravenously with each peptide (100 μg in 100 μl of PBS), whereas control group—with 100 σl of PBS. Different groups of mice were bled at 0 min (no injections), 90, and 180 min after injections, plasma was obtained, combined within each group, and subjected to FPLC analysis. Cholesterol distribution among the different lipoprotein classes was assessed and cholesterol content (Act*ml) or area under the VLDL, IDL/LDL, and HDL peaks was quantified. The data are expressed as percent of change of total cholesterol (TC) content of VLDL, IDL/LDL, and HDL classes in experimental mice relative to control (PBS injected) mice. The results are presented in Tables 6 and 7. It can be seen in Tables 6 and 7 that SEQ ID NO: 1, which belongs to N-terminal part of Helix 6, has the strongest impact on plasma levels of low density lipoproteins compare to other sequences, whereas SEQ ID NO: 7, which belongs to the C-terminal part of Helix 6, demonstrated the marked HDL-C elevating properties.

### Effect of Frame-Shift Derived Peptides on Plasma Lipoprotein Profile. 90 Min After Single Bolus Injections (SBI)

TABLE 6

| | | | | | | |
|---|---|---|---|---|---|---|
| # | AVP# | Sequence | M.W. | VLDL | IDL/LDL | HDL |

| | | | | | | |
|---|---|---|---|---|---|---|
| 1 | 606 | YEFRDRMRTH | 1451.8 | −34 | −26 | −5.3 |
| 2 | 13577 | PVAEEFRDRMRTHVDSLRTQLAP | 2766.5 | 0 | −12.4 | −1.6 |
| 3 | 13578 | EEFRDRMRTHVDSLRTQLAP | 2499.1 | 38.4 | 12.2 | −15.1 |
| 4 | 13579 | FRDRMRTHVDSLRTQLAP | 2240.8 | −4 | −3 | 19.5 |
| 5 | 13580 | RDRMRTHVDSLRTQLAP | 2093.4 | −3.5 | 12.6 | 11.1 |
| 6 | 13590 | MRTHVDSLRTQLAP | 1665.9 | −1.6 | −2.1 | −6.3 |
| 7 | 13581 | RTHVDSLRTQLAP | 1534.7 | −6.5 | 5.2 | 9.1 |
| 8 | 13582 | THVDSLRTQLAP | 1376.6 | 26 | 2.3 | 31 |
| 9 | 13583 | DSLRTQLAP | 1041.2 | 9.3 | 10.4 | 8.3 |
| 10 | 13600 | PVAEEFRDRMRTHVDSLR | 2255.4 | 47 | 23.5 | 16.1 |
| 11 | 13584 | PVAEEFRDRMRTHV | 1783.9 | 0 | 9.7 | −22.3 |
| 12 | 13585 | PVAEEFRDRMR | 1446.3 | −4 | 6.6 | 5 |
| 13 | 13586 | PVAEEFRDRM | 1290.2 | −21 | 2.1 | 13.5 |
| 14 | 13587 | PVAEEFRDR | 1159.3 | −10 | 8.5 | 11.4 |
| 15 | 13588 | PVAEEF | 731.8 | 30.7 | 0 | 20 |
| 16 | 13589 | PVAEE | 584.6 | 4.4 | 4.6 | 16.5 |

Mice were divided into two groups (4 mice in each group). Peptides or PBS were injected intravenously into experimental or control mice, respectively. At 90 min after SBI plasma was obtained, combined within each group, and applied on Superose 6 column. Data are expressed as % of change of TC ¦Area (Act*ml) or area under the VLDL, IDL/LDL, and HDL peaks compare to PBS control.

### Effect of Frame-Shift Derived Peptides on Plasma Lipoprotein Profile. 180 Min After Single Bolus Injections (SBI)

TABLE 7

| | | | | | | |
|---|---|---|---|---|---|---|
| # | AVP# | Sequence | M.W. | VLDL | IDL/LDL | HDL |

| | | | | | | |
|---|---|---|---|---|---|---|
| 1 | 606 | YEFRDRMRTH | 1451.8 | −29.31 | −34.11 | −10 |
| 2 | 13577 | PVAEEFRDRMRTHVDSLRTQLAP | 2766.5 | 19.5 | 3.1 | 4.3 |
| 3 | 13578 | EEFRDRMRTHVDSLRTQLAP | 2499.1 | −18.8 | −6.3 | −15.7 |
| 4 | 13579 | FRDRMRTHVDSLRTQLAP | 2240.8 | −5 | −7.1 | −9.5 |
| 5 | 13580 | RDRMRTHVDSLRTQLAP | 2093.4 | −21.5 | −7.8 | 4.9 |
| 6 | 13590 | MRTHVDSLRTQLAP | 1665.9 | −2.8 | −15.3 | 0 |
| 7 | 13581 | RTHVDSLRTQLAP | 1534.7 | −17.6 | 0 | −14.8 |
| 8 | 13582 | THVDSLRTQLAP | 1376.6 | −25.7 | −11.1 | 29.4 |
| 9 | 13583 | DSLRTQLAP | 1041.2 | 10.8 | 12.9 | −10.1 |
| 10 | 13600 | PVAEEFRDRMRTHVDSLR | 2255.4 | 0 | 6.2 | 8.4 |
| 11 | 13584 | PVAEEFRDRMRTHV | 1783.9 | −20.7 | −4.3 | −24.1 |
| 12 | 13585 | PVAEEFRDRMR | 1446.3 | −17 | −3.2 | −4.5 |
| 13 | 13586 | PVAEEFRDRM | 1290.2 | 23.3 | 18.8 | 4.5 |
| 14 | 13587 | PVAEEFRDR | 1159.3 | −33.7 | 5.1 | 0 |
| 15 | 13588 | PVAEEF | 731.8 | 51 | 14.8 | 8.8 |
| 16 | 13589 | PVAEE | 584.6 | 9.7 | 7.5 | 7.2 |

Mice were divided into two groups (4 mice in each group). Peptides or PBS were injected intravenously into experimental or control mice, respectively. At 180 min after SBI plasma was obtained, combined within each group, and applied on Superose 6 column. Data are expressed as % of change of TC ¦Area (Act*ml) or area under the VLDL, IDL/LDL, and HDL peaks relative to PBS control.

Effect of chemically modified derivatives of SEQ ID NO: 1 and peptides derived from other mouse apoAl regions on mouse plasma lipoprotein profile (Single bolus injections)—To increase SEQ ID NO: 1 hydrophobicity, Phenyl Acetyl or Pivalic Acid were attached to N-Terminal Tyr residue of SEQ ID NO: 1, or Tyr residue was removed from N-terminus. These peptides were tested for possible effect on cholesterol metabolism of mildly hyperlipidemic C57BL/6J mice fed a cholate-containing high fat diet (HFC). Mice were divided into two groups—control and experimental. Each group contained 4 mice. Experimental mice were injected intravenously with each peptide (100 μg in 100 μl of PBS), whereas control group—with 100 μl of PBS. Different groups of mice were bled at 0 min (no injections), 90, and 180 min after injections, plasma was obtained, combined within each group, and subjected to FPLC analysis. Cholesterol distribution among the different lipoprotein classes was assessed and cholesterol content (Act*ml) or area under the VLDL, IDL/LDL, and HDL peaks was quantified. The data are expressed as percent of change of total cholesterol (TC) content of VLDL, IDL/LDL, and HDL classes in experimental mice relative to control (PBS injected) mice. The results are presented in Table 8. It can be seen in the table that all above modifications did not result in increase of SEQ ID NO: 1 potency. The few peptides belonging to other regions of mouse apolipoprotein Al were also tested in this model and did not demonstrate any remarkable activity (Table 8).

### Effect of Chemically Modified Derivatives of AVP#606 and Peptides Derived from Other Mouse ApoA1 Regions on Mouse Plasma Lipoprotein Profile. Single Bolus Injections

TABLE 8

| | | | | | | |
|---|---|---|---|---|---|---|
| # | AVP# | Sequence | M.W. | VLDL | IDL/LDL | HDL |

90 minutes after SBI

| | | | | | | |
|---|---|---|---|---|---|---|
| 1 | 13625 | Ph_YEFRDRMRTH | 1527.7 | 31.7 | 3.1 | −4.5 |
| 2 | 25740 | Piv_YEFRDRMRTH | 1494 | 11 | −1.3 | 5.7 |
| 3 | 25984 | EFRDRMRTH | 1288.3 | 4.4 | 0 | −11.2 |
| 4 | 13608 | WDKVKDF | 978.1 | −17.4 | −16.1 | −12.4 |
| 5 | 13609 | SGRDYVSQFES | 1315.4 | 21.4 | 8.2 | 0 |
| 6 | 13610 | YLDEFQKKWKE | 1554.8 | 9.5 | 11.7 | 19.5 |
| 7 | 13611 | TRDFWDNLEKETDW | 1896.1 | 32.6 | 11.7 | 9.5 |
| 8 | 13616 | WDKVKDFANVYVDA | 2053.3 | −2.6 | 9 | 0 |
| | | VKD | | | | |

180 minutes after SBI

| | | | | | | |
|---|---|---|---|---|---|---|
| 1 | 13625 | Ph_YEFRDRMRTH | 1527.7 | −17.1 | −31.2 | −21.5 |
| 2 | 25740 | Piv_YEFRDRMRTH | 1494 | −14.9 | −10.8 | −7.4 |
| 3 | 25984 | EFRDRMRTH | 1288.3 | 8.9 | −11 | −3 |
| 4 | 13608 | WDKVKDF | 978.1 | −15.6 | 15.5 | −11.7 |
| 5 | 13609 | SGRDYVSQFES | 1315.4 | 18.4 | 0 | 0 |
| 6 | 13610 | YLDEFQKKWKE | 1554.8 | −10.6 | −8.5 | −20 |
| 7 | 13611 | TRDFWDNLEKETDW | 1896.1 | −2 | 0 | −16.1 |
| 8 | 13616 | WDKVKDFANVYVDA | 2053.3 | 19.4 | 19.9 | 18.1 |
| | | VKD | | | | |

Mice were divided into two groups (4 mice in each group). Peptides or PBS were injected intravenously into experimental or control mice, respectively. At 90 and 180 min after SBI plasma was obtained, combined within each group, and applied on Superose 6 column. Data are expressed as % of change of TC ¦Area (Act*ml) or area under the VLDL, IDL/LDL, and HDL peaks relative to PBS control.

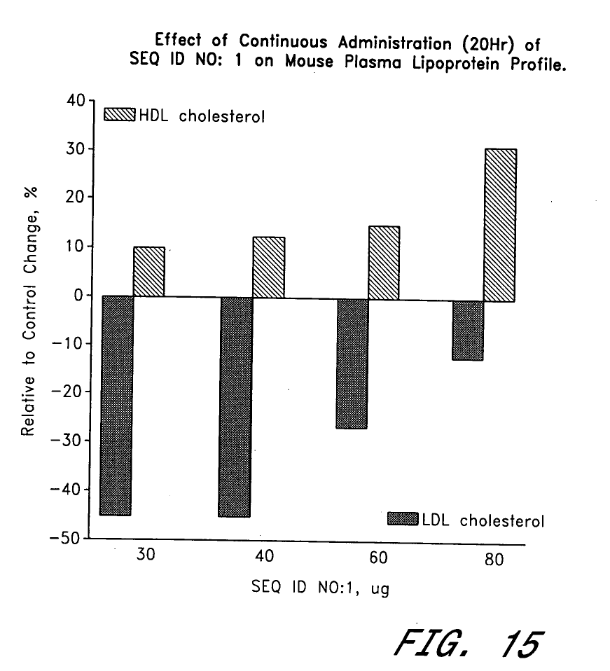

Impact of SEQ ID NO: 1 on lipoprotein metabolism (20 hrs pumps)—To determine relatively long-term effect of SEQ ID NO: 1 on plasma lipoprotein profile, the Alzet Mini-Osmotic pumps, containing SEQ ID NO: 1 or PBS, were surgically inserted in canulated Chow fed C57BL/6J mice. The pumps flow rate was equal to 8 ul per hr, which provided the delivery amount of SEQ ID NO: 1 per hr as indicated for FIG. 9. Mice were switched on HFC diet immediately after surgery. 20 hrs later plasma samples were obtained, combined within each group (4 to 6 mice), and subjected to FPLC analysis and agarose gel electrophoresis for assessment of cholesterol and phospholipid distribution among the lipoprotein classes. The results are presented in FIG. 15 and show dramatic decrease in low density lipoproteins levels in plasma of mice, which received SEQ ID NO: 1, whereas the plasma HDL cholesterol levels in these mice were elevated. The LDL lowering effect was attenuated with increase of SEQ ID NO: 1 concentration. This phenomenon can be explained by self-competition between free and LDL-bound form of SEQ ID NO: 1 for the liver lipoprotein binding sites. In contrast, HDL cholesterol levels were positively correlated with SEQ ID NO: 1 concentration. The increase in plasma HDL-C indicates of possible activation of LCAT (lecithin:cholesterol acyltransferase) or increased production of ApoA-I, whereas elevated plasma levels of HDL phospholipids (data not shown) suggest involvement of PLTP (phospholipid transfer protein) in the mechanism of SEQ ID NO: 1 action. Thus, obtained data indicate SEQ ID NO: 1 multi-functionality and suggest its involvement in both HDL and LDL pathways.

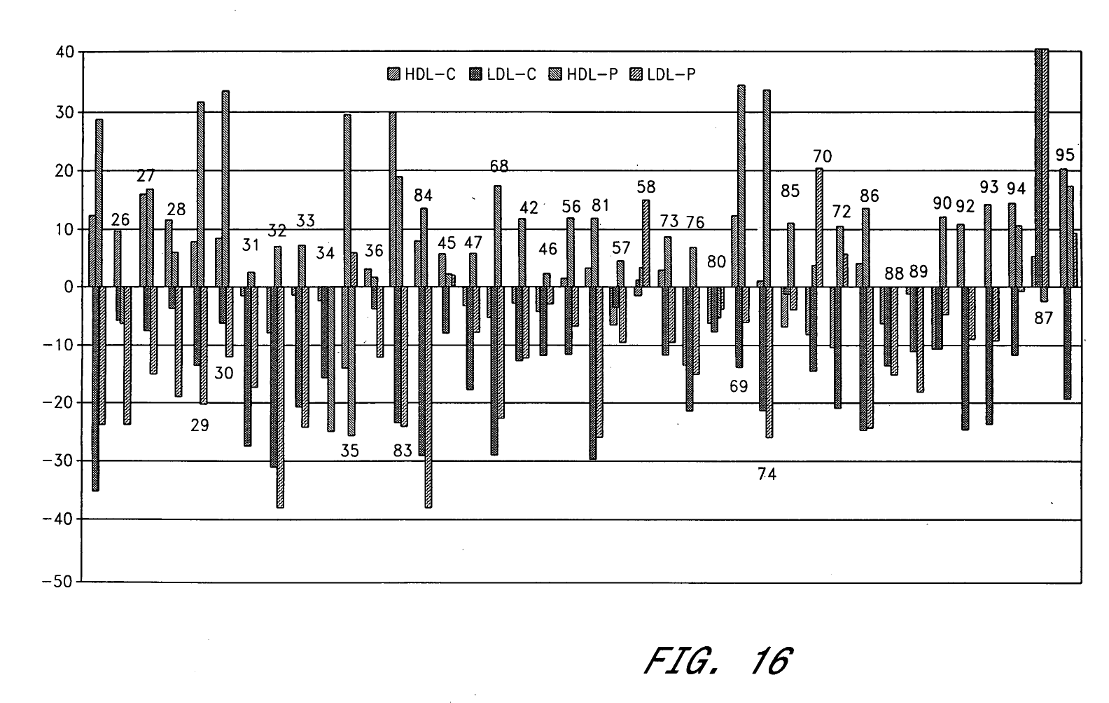

Impact of SEQ ID NO: 1 structural derivatives (3 to 13 amino acid residues) on lipoprotein metabolism (20 hrs pumps)—To determine relatively long-term effect on plasma lipoprotein profile, the Alzet Mini-osmotic pumps, containing SEQ ID NO: 1 derivatives or PBS, were surgically inserted in canulated Chow fed C57BL/6J mice. The pumps flow rate was equal to 8 ul per hr, which provided the deliveries of 30-40 μg of peptides per hr. Mice were switched on HFC diet immediately after surgery. 20 hrs later plasma samples were obtained, combined within each group (4 to 6 mice), and subjected to FPLC analysis and agarose gel electrophoresis for assessment of cholesterol and phospholipid distribution among the lipoprotein classes. The data for HDL-C are presented in FIG. 16 and demonstrate significant decrease of plasma levels of low density lipoproteins in peptides treated mice, whereas the plasma HDL cholesterol levels in these mice were elevated. However, few peptides (such as SEQ ID NOs: 35 and 87) caused the opposite effect, i.e., increase of the plasma levels of low density lipoproteins and decrease in high density lipoproteins cholesterol and phospholipids. This reversal of effect is valuable information for SAR purposes in light of close structural relationships of these “clearance blocking” peptides with “clearance enhancing” peptides. The modest increase in plasma HDL-C might indicate on possible activation of LCAT (lecithin: cholesterol acyltransferase) or increased production of ApoA-I, whereas elevated plasma levels of HDL phospholipids suggest involvement of PLTP (phospholipid transfer protein) in the mechanism of peptides action. Thus, obtained data suggest that SEQ ID NO: 1 derivatives are involved in both HDL and LDL pathways.

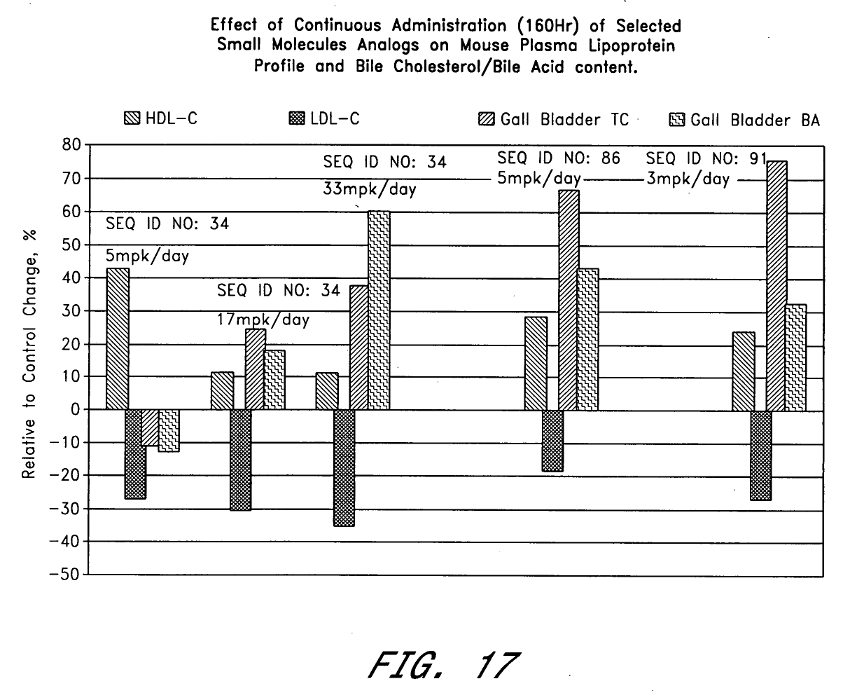

Long term effect of SEQ ID NO: 34 (“template”, 3 amino acid residues peptide) and its modified derivatives (SEQ ID Nos: 8 and 91) on lipoprotein metabolism (7 days pumps)—To determine long-term effect of these peptides on plasma lipoprotein profile, the Alzet Mini-osmotic pumps, containing peptides or PBS, were surgically inserted in canulated Chow fed C57BL/6J mice. The pumps flow rate was equal to 1 μl per hr, which provided the deliveries of indicated in FIG. 17 amount of each peptide per hr. Mice were switched on HFC diet immediately after surgery. 160 hrs later mice were sacrificed, plasma samples were obtained, combined within each group (4 to 6 mice), and subjected to FPLC analysis and agarose gel electrophoresis for assessment of cholesterol and phospholipid distribution among the lipoprotein classes. Bile was immediately removed from gall bladders and total cholesterol/bile acids contents were determined as described in methods. The data are presented in FIG. 17 and demonstrate significant decrease of plasma levels of low density lipoproteins in peptides treated mice, elevation of plasma HDL cholesterol (and HDL phospholipid-data not shown), and dramatic increase of Gall Bladder total cholesterol and bile acids amount. The modest increase in plasma HDL-C might indicate on possible activation of LCAT (lecithin: cholesterol acyltransferase) or increased production of ApoA-I, whereas elevated plasma levels of HDL phospholipids suggest activation of PLTP (phospholipid transfer protein), which is known to be involved in maintenance of plasma HDL levels and generating nascent HDL particles (primary cholesterol acceptors). It can be seen in FIG. 17 that administration of SEQ ID Nos: 34, 86 and 91 into mice resulted in an increase in the amount of phospholipid in the HDL zone, which is usually accompanied by an increase in total cholesterol and bile acids recovered from the gall bladder. The increased amount of cholesterol/bile acids in a Gall Bladder, along with decrease of plasma low density lipoprotein levels (VLDL, IDL, LDL) and increase of the HDL levels, indicates on enhanced “Plasma >Liver >Gall Bladder” cholesterol flux, i.e., reverse cholesterol transport in presence of the peptides in accordance with preferred aspects of the present invention, as illustrated in FIG. 17.

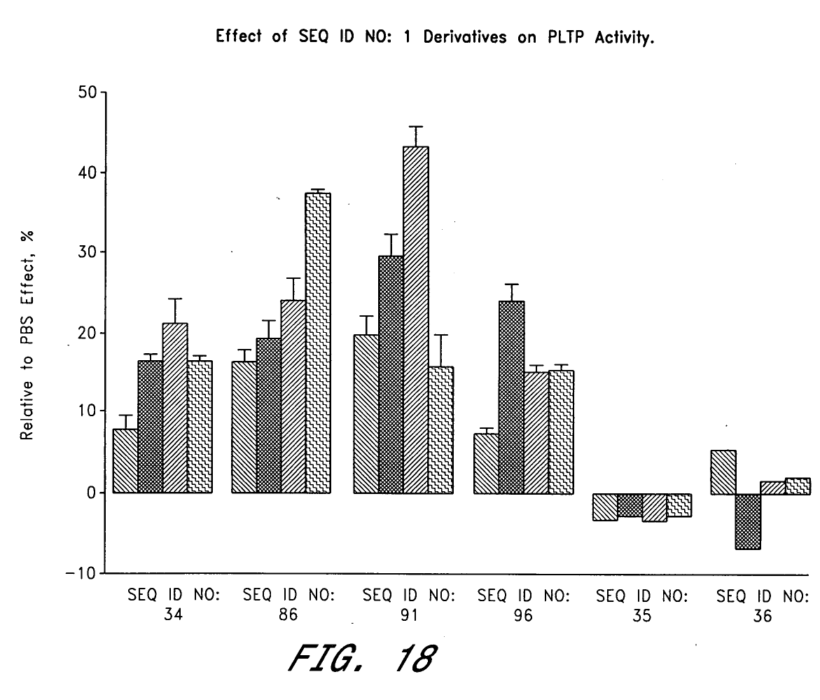

Effect of peptides on PLTP activity—It can be seen in FIG. 18 that incubation of mouse plasma with SEQ ID Nos: 34, 86, 91 and 96 resulted in activation of PLTP. Mouse plasma (source of enzyme), obtained from Chow fed mice and from mice fed with high fat cholate containing diet for 4 days, was incubated with PBS or the peptide of SEQ ID Nos: 34, 86, 91 and 96 (0.4, 2, 5, and lOug) at RT for 30 min. Reaction was started by addition of 0.3 μl of plasma/PBS or plasma/SEQ ID NO mixture to 100 μl of assay solution containing fluorescent substrate. PLTP activity was monitored on Spectrofluorometer for 20 min at 37° C. Each bar represents the Mean±SEM obtained from 3 independent experiments. These data are in a good agreement with results of studying the long-term effect of these peptides on mouse plasma HDL levels (e.g., the results obtained with 7 days pumps, shown in FIG. 17) and provide strong evidence in favor of PLTP involvement in in vivo mechanisms of peptide action. Peptides corresponding to SEQ ID Nos: 35 and 36 did not have any significant effect on PLTP activity. It is noteworthy that these two peptides also did not show significant activity upon infusion into mice for 20 hrs (see FIG. 16).

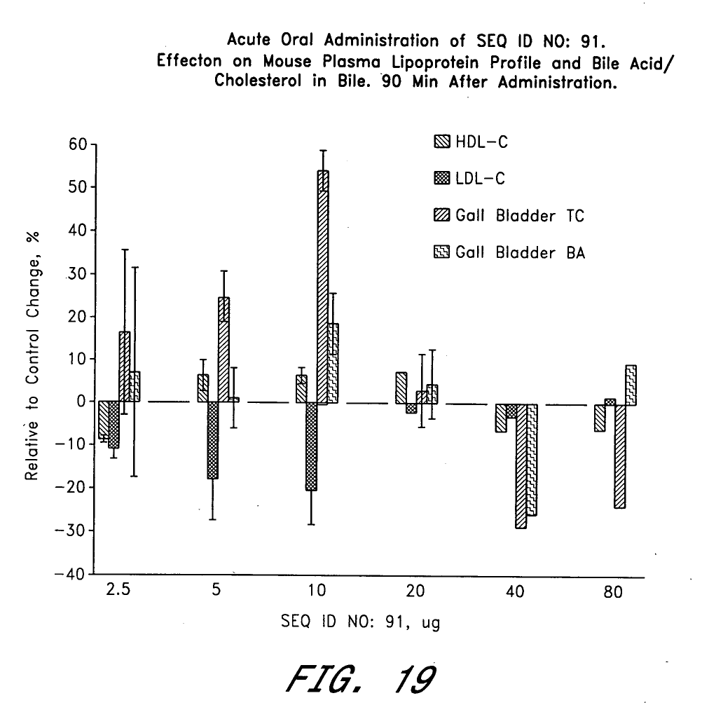

Effect of Acute Oral Administration of SEQ ID NO 91 (A VP-26249) on Mouse Plasma Lipoprotein Profile and Bile Acid/Cholesterol in Bile—With reference to FIG. 19, bars shown for 2.5 and 20 ug represent mean±SEM of 2 independent experiments and bars shown for 5 and 10 ug represent mean±SEM of 3 independent experiments with C57BL/6J fed HFD. No error bars—single experiment. Effect is expressed as % of change relative to PBS control (0%).

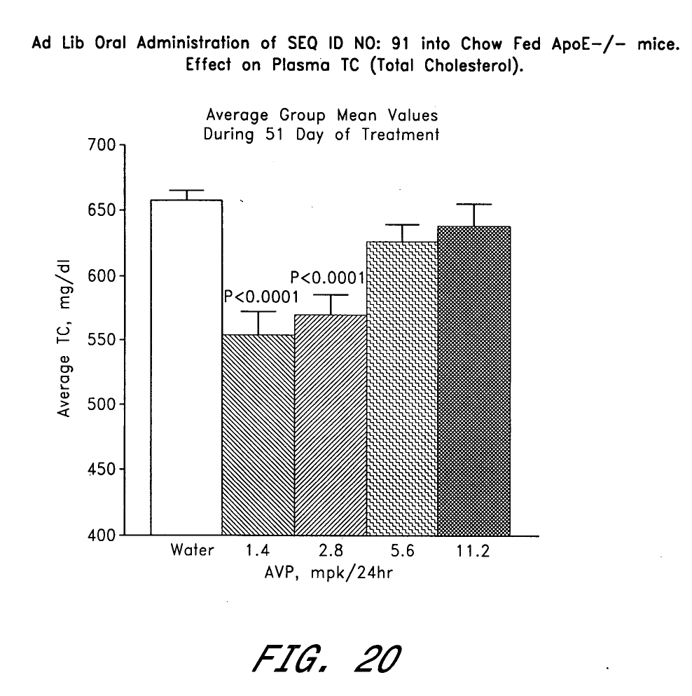

Effect ofAd Lib Oral Administration of SEQ ID NO: 91 (A VP-26249) into Chow Fed ApoE−/− mice on Plasma TC (Total Cholesterol)—With reference to FIG. 20, two months old ApoE−/− male mice were fed Chow diet. On a day of the experiment at T0 (Time 0) mice were bled (50 ul), plasma TC was determined, and mice were divided into groups (4 mice per group) with similar TO group mean cholesterol values. Control mice received drinking water and experimental mice received water solution of compound. Mice were bled every 72-96 hr, and plasma cholesterol was quantified. Each bar represents mean±SEM of 4 animals and is representative of 15 determinations carried out biweekly. Amount of SEQ ID NO: 91 is expressed as mg per kg (mpk), consumed per 24 hr.

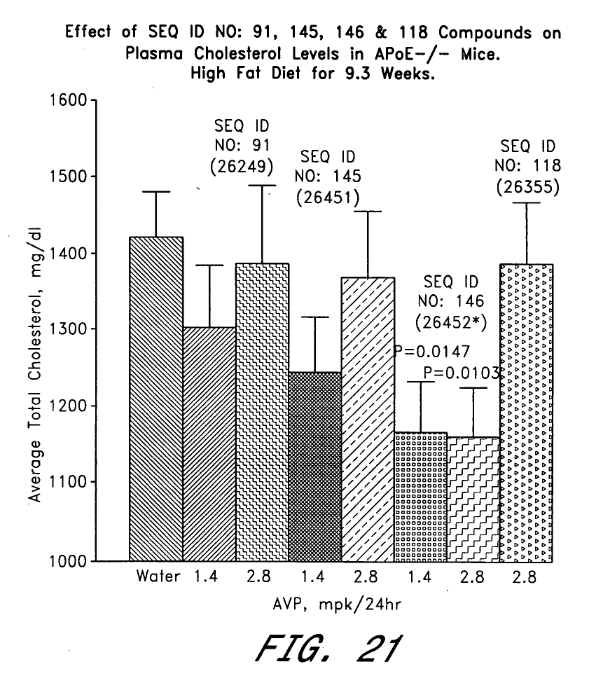

Effect of SEQ ID NOS: 91, 145, 146 and 118 (A VP-26249, 26451, 26452, and 26355, respectively) on Plasma Cholesterol Levels in ApoE−/− Mice Fed High Fat Diet—With reference to FIG. 21, three months old ApoE−/− male mice were maintained on Chow diet. On day 0, mice were bled, plasma TC was determined, and mice were divided into groups (4 mice per group) with similar group mean cholesterol and weight values. Control mice received drinking water and experimental mice received water solution of compounds. Four weeks later, mice were switched on high fat diet. Mice were bled once in a 10 days, and total plasma cholesterol was quantified. Each bar represents the mean±SEM of 4 animals and is representative of 7 determinations. Amount of SEQ ID NO is expressed as mg per kg (mpk), consumed per 24 hr.

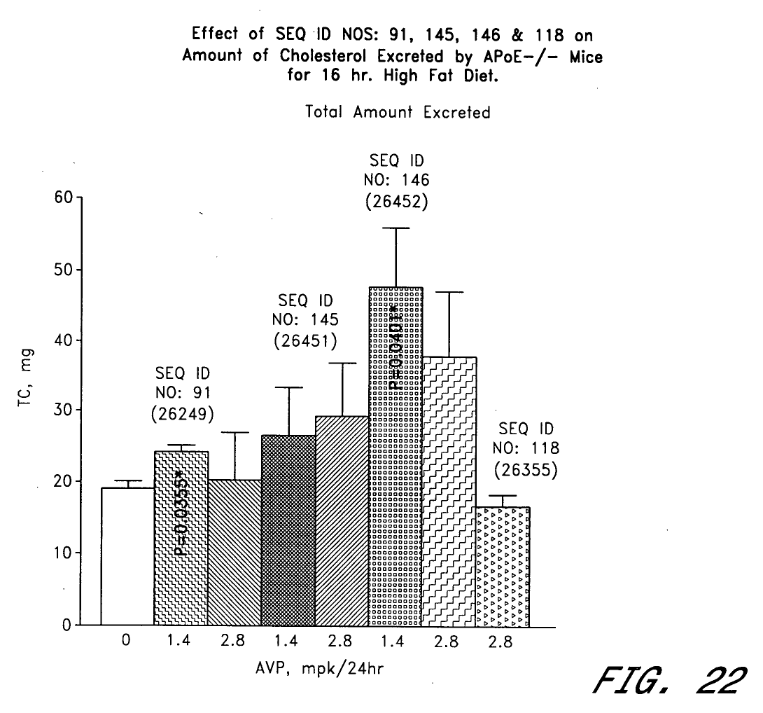

Effect of SEQ ID NOS: 91, 145, 146 and 118 (AVP-26249, 26451, 26452, and 26355, respectively) on Amount of Cholesterol Excreted by ApoE−/− Mice Fed High Fat Diets—With reference to FIG. 22, eight groups of 5 months old ApoE−/− male mice (4 mice in each group) were maintained on high fat diet. Control mice received drinking water, whereas experimental mice received water solutions of compounds “Ad Lib”. Mice were placed in metabolic cages overnight. Next day, feces were collected, dried for 72 hr, weighed, and total cholesterol was extracted and quantified. Each bar represents the mean±SEM of two independent “metabolic cage” experiments that were performed 2.4 and 3.4 weeks after the mice were fed high fat diet. Amount of SEQ ID NO is expressed as mg per kg (mpk), consumed per 24 hr.

In conclusion, the results summarized in FIGS. 2-22 and Tables 6-8 demonstrate that hyperlipidemic mice administered the peptide mediators of RCT designed in accordance with the Molecular Model of the present invention, via either intravenous or oral delivery routes, exhibited substantial improvements in plasma lipoprotein profile, which are due to enhanced clearance of low density (atherogenic) lipoproteins and elevation of plasma levels of antiatherogenic high density lipoproteins.

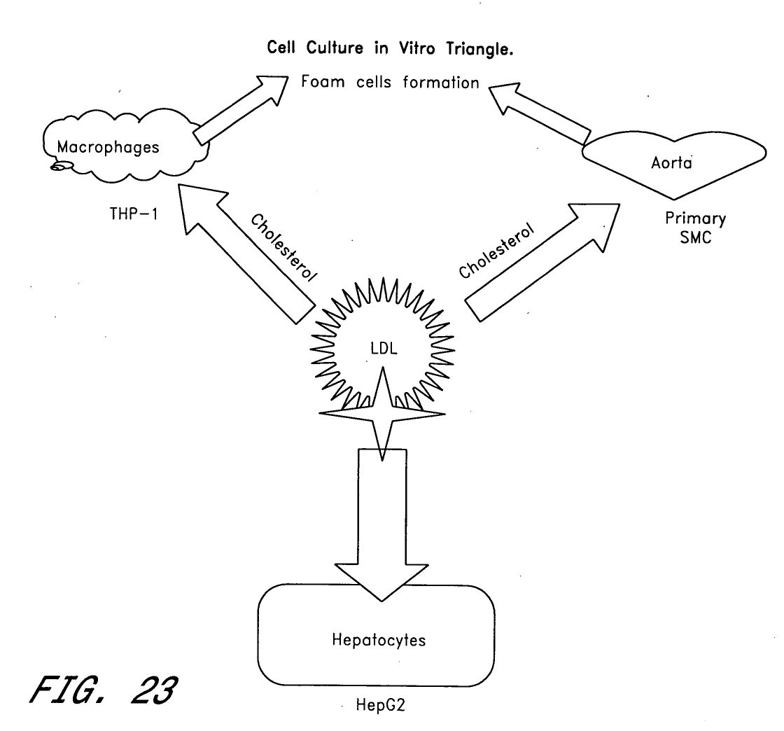

With reference to FIG. 23, a schematic diagram shows an in vitro triangle used in a screening method to identify test compounds likely to enhance RCT in vivo. The cultured macrophage cells are used to assess the effects of test RCT mediator compounds on both ac-LDL cholesterol accumulation and cholesterol efflux from pre-loaded macrophage cells (macrophages which accumulate cholesterol contribute to foam cell formation and atherosclerotic plaque formation). Thus, this compartment of the triangle is used to evaluate the effectiveness of test compounds on RCT as well as pathogenesis of atherosclerosis. The cultured primary smooth muscle cells are used to assess the effects of test RCT mediator compounds on ox-LDL accumulation in the vascular wall, which may also be related to the formation of foam cells and the progression of atherosclerosis. The cultured hepatocytes are used to assess the effects of the test RCT mediator compounds on cholesterol uptake by the liver. The use of the peripheral cells (macrophages and/or smooth muscle cells in combination with the liver cells) advantageously provides a monitor for RCT—reduced cholesterol accumulation and enhanced cholesterol efflux from the peripheral cells, as well as uptake by the liver (for metabolism and excretion).

Effect of SEQ ID NOS: 91 and 146 (A VP-26249 and A VP-26452, respectively) on LDL Mediated Accumulation of Total Cholesterol in HepG2 Cells. With reference to FIG. 24, human HepG2 cells were plated in 24 wells plate at the density 2.5×105 per well in serum free (lipoprotein free) assay media. 48 hrs later 25 ug of human LDL pre-incubated for lhr at RT with PBS or compounds, were added to the cells in 500 ul of serum free media. Treated cells were incubated for 24 hr at 37° C. in a humidified 5% CO2 incubator. Each bar represents the Mean±SEM of 2-3 independent experiments.

Effect of SEQ ID NO: 91 (A VP-26249) on Ac-LDL Mediated Accumulation of Total Cholesterol and Cholesteryl Ester in Macrophages. With reference to FIG. 25, human THP-1 cells were plated in assay media in 24 wells plate at the density 1×106 per well in presence of 5×10−8 M of PMA. 48 hrs later 50 ug of human AcLDL, pre-incubated for lhr at RT with PBS (control), or AVP-26249, was added to the cells in 500 ul of assay media. Treated cells were incubated for 24 hr at 37° C. in a humidified 5% CO2 incubator. Each bar represents the Mean±SEM of 3-9 replicates.

Effect of SEQ ID NOS. 91 and 146 (A VP-26249 and 26452, respectively) on Ox-LDL Mediated Accumulation of Total Cholesterol and Cholesteryl Ester in Vascular Smooth Muscle Cells. With reference to FIG. 26, human vascular smooth muscle cells were plated in 24 wells plate at the density 9×104 per well in serum free assay media. 24 hrs later PBS only or human oxidized LDL, pre-incubated for 1 hr at RT with PBS, or with compounds, were added to the cells in 500 ul of assay media. Treated cells were incubated for 24 hr at 37° C. in a humidified 5% CO2 incubator. Each bar represents the Mean±SEM of 4-7 independent experiments.

Effect of SEQ ID NOS: 91 and 146 (A VP-26249 and A VP-26452, respectively) on Cholesterol Efflux from AcLDL-Loaded Macrophages. With reference to FIG. 27, human THP-1 cells were plated in assay media in 24 wells plate at the density 1×106 per well in presence of 5×10−8 M of PMA. 48 hrs later 50 ug of human acetylated LDL or PBS were added to the cells in 500 ul of assay media containing PMA. 24 hrs later cells were washed and PBS or compounds, were added to the cells in 500 ul of assay media. Before and after each treatment cells were incubated at 37° C. in a humidified 5% CO2 incubator. Each bar represents the Mean±SEM of 5-6 independent experiments.

One goal is to move cholesterol from the macrophages and aorta cells into the liver for cholesterol clearance (See FIG. 30). The ability of one of the test compounds such as those shown in Tables 3-5 to effect RCT may be predicted based upon the assays performed as shown in FIGS. 24-27 above. That is, these three cell types provide a snapshot view of cholesterol status within the organism. The ability of a given compound to decrease levels of cholesterol and CE in a macrophage cell such as THP-1 cells and vascular smooth muscle cells while increasing cholesterol levels in hepatocytes such as (HepG2 cells) is predictive of its effectiveness in vivo. Accordingly, one embodiment of the present invention involves a screening method in which test compounds are assayed in vitro using macrophage, smooth muscle and liver cell lines such as those disclosed, to provide an indication of in vivo RCT.

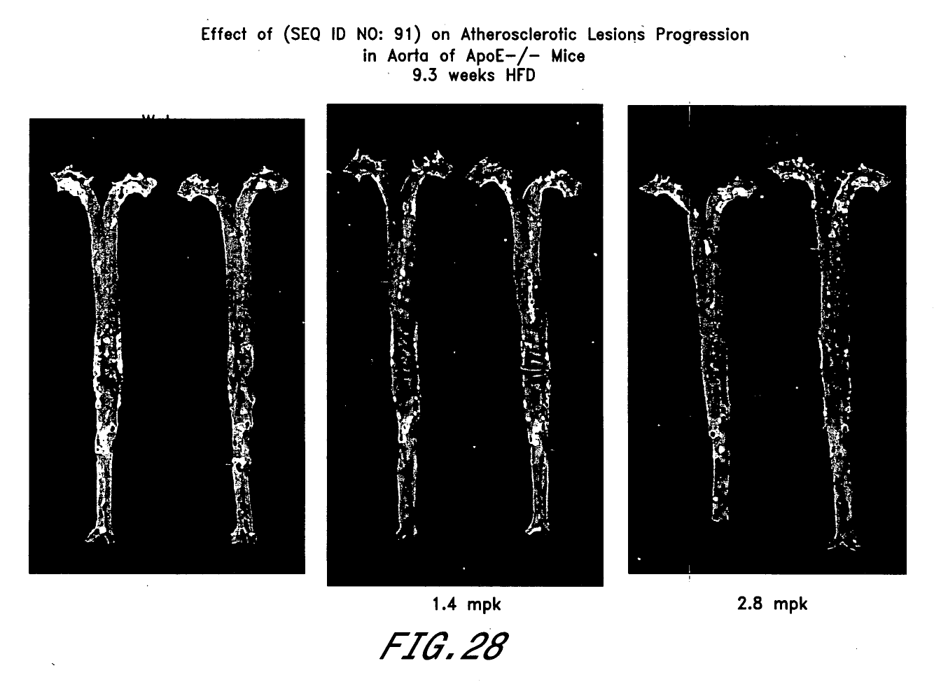

Effect of SEQ ID NO: 91 (A VP-26249) on Atherosclerotic Lesions Progression in Aorta of ApoE−/− Mice Fed High Fat Diets With reference to FIG. 28, ApoE−/− male mice were maintained on Chow diet for 4 weeks and on HFD (1.25% of cholesterol) for 9.3 weeks. Mice received SEQ ID NO: 91 (AVP-26249) “ad lib” via the drinking water for 13.3 weeks. The concentrations of SEQ ID NO: 91 are zero (left panel), 1.4 μg/kg (middle panel) and 2.8 μg/kg (right panel). At euthanasia, animals were perfused with PBS, followed by formal-sucrose (4% paraformaldehyde and 5% sucrose in PBS, pH 7.4). The entire mouse aorta was dissected from the proximal ascendig aorta to the bifurcation of the iliac artery by using a dissecting microscope. Adventitial fat was removed and the artery was opened longitudinally, pinned flat onto black dissecting wax, stained with Sudan IV, and photographed at a fixed magnification. The photographs were digitized and the digital images are shown. Total aortic area and aortic lesion area were calculated by using Adope Photoshop 7.0 and NIH Scion Image software (data not shown).

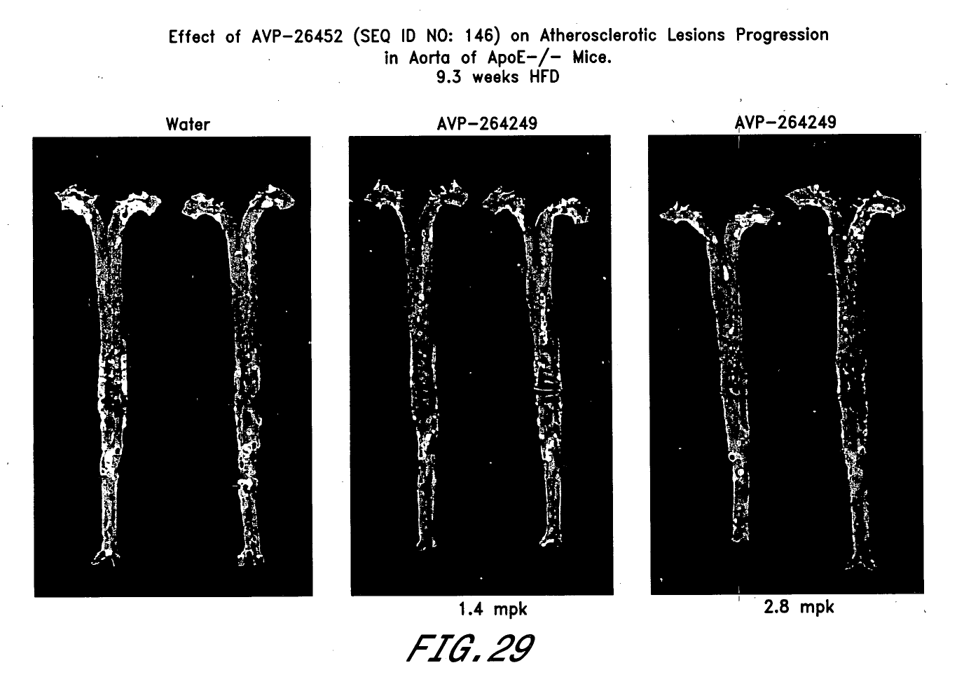

Effect of SEQ ID NO: 146 (A VP-26452) on Atherosclerotic Lesions Progression in Aorta of ApoE−/− Mice Fed High Fat Diets With reference to FIG. 29, ApoE−/− male mice were maintained on Chow diet for 4 weeks and on HFD (1.25% of cholesterol) for 9.3 weeks. Mice received SEQ ID NO: 146 (AVP-26452) “ad lib” via the drinking water for 13.3 weeks. The concentrations of SEQ ID NO: 146 are zero (left panel), 1.4 μg/kg (middle panel) and 2.8 μg/kg (right panel).

### REFERENCES CITED (AND INCORPORATED HEREIN BY REFERENCE THERETO)

Alam et al. (2001) J. Biol. Chem. 276, 15641-15649
Anantharamaiah et al. (1985) J. Biol. Chem. 260, 10248-10255
Anantharamaiah et al. (1987) J. Lipid Res. 29, 309-318
Anantharamaiah et al. (1990) Arteriosclerosis, 10, 95-105
Arai et al. (1999) J. Biol. Chem. 274, 2366-2371
Argraves et al. (1997) J. Clin. Invest. 100, 2170-2181
Austin et al. (1988) JAMA, 260, 1917-1921
Austin et al. (1990) Circulation, 82, 495-506
Banka et al. (1994) J. Biol. Chem. 269, 10288-10297
Barbaras et al. (1987) Biochem. Biophys. Res. Commun. 142, 63-69
Barter P (2000) Arterioscl. Thromb. Vasc. Biol. 20, 2029
Berneis and Krauss (2002) J. Lipid Res. 43, 1363-1379
Bhatnagar A. (1999) in Lipoproteins and Health Disease, pp. 737-752, Arnold, Loudon
Bolibar et al. (2000) Thromb. Haemost. 84, 955-961
Boren et al. (2001) J. Biol Chem. 276, 9214-9218
Brouillette and Anantharamaiah (1995) Biochim. Biophys. Acta. 1256, 103-129
Brunzell J D (1995) in The Meatbolic and Molecular Bases of Inherited Disorders, pp. 1913-1932, McGraw-Hill, Inc., New York
Buchko et al. (1996) J. Biol. Chem. 271, 3039-3045
Camejo et al. (1985) Atherosclerosis, 55, 93-105
Campos et al. (1992) Arteriosclerosis Thrombosis, 12, 187-193
Canner et al. (1986) JACC, 8, 1245-1255
Cao et al. (2002) J. Biol. Chem. 277, 39561-39565
Castelli et al. (1986) JAMA, 256, 2835-2838
Castro and Fielding (1998) Biochemistry, 27, 25-29
Chait et al. (1993) Am. J. Med. 94, 350-356
Chambenoit et al. (2001) J. Biol. Chem. 276, 9955-9960;
Chang et al. (1997) Annu Rev Biochem. 66, 613-638
Chapman et al. (1998) Eur Heart J., Suppl A: A24-30
Chen and Albers (1985) Biochim Biophys. Acta, 836, 275-285
Chen et al. (2000) J. Biol. Chem. 275, 30794-30800
Cohen et al. (1999) Curr Opin Lipidol. 10, 259-268
Collet et al. (1997) J Lipid Res. 38, 634-644
Collet et al. (1999) J. Lipid Res. 40, 1185-1193
Curtiss and Boisvert (2000) Curr. Opin. Lipidol. 11, 243-251
Datta et al. (2001) J. Lipid Res. 42, 1096-1104
Davis et al. (2002) J. Lipid Res. 43, 533-543
de Graaf et al. (1993) J. Clin. Endocrinol. Metab. 76, 197-202
Downs et al. (1998) JAMA, 279, 1615-1622
Duverger et al. (1996) Circulation, 94, 713-717
Ehnholm et al. (1998) J. Lipid Res. 39, 1248-1253
Epand et al. (1987) J. Biol. Chem. 262, 9389-9396
Eriksson et al. (1999) Circulation, 100, 594-598
Fan et al. (2001) J. Biol. Chem. 276, 40071-40079
Fidge N H (1999) J. Lipid Res. 40, 187-201
Fielding et al. (1994) Biochemistry, 33, 6981-6985
Fitch W M (1977) Genetics, 86, 623-644
Fogelman et al. (2003) U.S. patent application Publication, US 2003/0045460 A1 Foger et al. (1996) Arterioscler Thromb Vasc Biology, 16, 1430-1436
Foger et al. (1999) J. Biol. Chem. 274, 36912-36920
Frank and Marcel (2000) J. Lipid Res. 41, 853-872
Frick et al. (1987) N. England J Medicine, 317, 1237-1245
Fuskushima et al. (1980) J Biol. Chem. 255, 10651-10657
Gamble et al. (1978) J. Lipid Res. 16,1068-1070
Garber et al. (1992) Arteriosclerosis and Thrombosis, 12, 886-894
Garber et al. (2001) J. Lipid Res. 42, 545-552
Garcia et al. (1996) Biochemistry, 35, 13064-13071
Genest et al. (1991) Am J Cardiol. 67, 1185-1189
Genest et al. (1992) Circulation, 85, 2025-2033
Genest et al. (1999) J. Invest. Med. 47, 31-42
Gibbons et al. (1995) Am. J. Med. 99, 378-385
Gillotte et al. (1999) J. Biol. Chem. 274, 2021-2028
Glomset J A (1968) J. Lipid Res. 9, 155-167
Goldberg I. (1996) J. Lipid Res. 37, 693-707
Golder-Novoselsky et al. (1995) Biochim. Biophys. Acta, 1254, 217-220
Goldstein and Brown (1974) J Biol. Chem. 249,5153-5162
Gordon et al. (1989) N Engl. J Med. 321, 1311-1315
Gotto A M (2001) Circulation, 103, 2213
Griffin et al. (1994) Atherosclerosis, 106, 241-253
Groen et al. (2001) J. Clin. Invest. 108, 843-850
Hajjar and Haberland (1997) J. Biol. Chem. 272, 22975-22978
Hara and Yokoyama (1991) J. Biol. Chem. 266, 3080-3086
Hedrick et al. (2001) J. Lipid Res. 42, 563-570
Huang et al. (1995) Arterioscler. Thromb. Vasc. Biology, 15, 1412-1418
Huang et al. (1997) Arterioscler. Thromb. Vasc. Biol. 17, 2010-2015
Hulley et al. (1998) JAMA, 280, 605-613
Huuskonen and Ehnholm (2000) Curr. Opin. Lipidol. 11, 285-289
Huuskonen et al. (2000) Atherosclerosis, 151, 451-461
Ikewaki et al. (1993) J. Clin. Invest. 92, 1650-1658
Ikewaki et al. (1995) Arterioscler. Thromb. Vasc. Biology, 15, 306-312
Ishigami et al. (1994) J. Biochem. (Tokyo) 116, 257-262
Jaakkola et al. (1993) Coron. Artery Dis. 4, 379-385
Jauhianen et al. (1993) J. Biol. Chem. 268, 4032-4036
Jiang et al. (1996) J. Clin. Invest. 98, 2373-2380
Jiang et al. (1999) J. Clin. Invest. 103, 907-914
Jonas A (1991) Biochim. Biophys, Acta, 1084, 205-220
Jones et al. (1998) Am. J. Cardiol. 81, 582-587
Kaiser and Kezdy (1983) Proc. Natl. Acad. Sci. USA, 80, 1137-1140
Kanellis et al. (1980) J. Biol. Chem. 255, 11464-11472
Kawano et al. (2000) J. Biol. Chem. 275, 29477-29481
Kozarsky et al. (2000) Arterioscler. Thromb. Vasc. Biology, 20, 721-727
Krauss and Burke (1981) J. Lipid Res. 23, 97-104
Krieger M. (1998) Proc. Natl. Acad. Sci. USA. 95, 4077-4080
La Belle and Krauss (1990) J. Lipid Res.,31, 1577-1588
Lindholm et al. (1998) Biochemistry, 37, 4863-4868
Liu and Krieger (2002) J. Biol. Chem. 277, 34125-34135
Lund-Katz et al. (1993) In “Peptides: Chemistry and Biology” (R. Haughten, ed.) ESCOM Press, Leiden, The Netherlands
Lusa et al. (1996) Biochem. J. 313, 275-282
Main et al. (1996) Biochim Biophys Acta, 29, 17-24
Marotti et al. (1993) Nature, 364, 73-75
Martin-Jadraque et al. (1996) Arch. Intern. Med. 156, 1081-1088
Marzal-Casacuberta et al. (1996) J. Biol. Chem. 271, 6720-6728
Matsumoto et al. (1997) J. Biol. Chem. 272, 16778-16782
McLachlan A D (1977) Nature, 267, 465-466
McLean et al. (1991) Biochemistry, 30, 31-37
McManus et al. (2000) J. Biol. Chem. 275, 5043-5051
Mendez et al. (1994) J Clin. Invest. 94, 1698-1705
Meng et al. (1995) J. Biol. Chem. 270, 8588-8596
Merkel et al. (2002) J. Lipid Res. 43, 1997-2006
Miccoli et al. (1997) J. Lipid Res. 38, 1242-1253
Miettinen et al. (1997) Arterioscler. Thromb. Vasc. Biology, 17, 3021-3032
Miller et al. (1987) Am Heart J. 113, 589-597
Milner et al. (1991) Biochim Biophys Acta, 26, 1082, 71-78
Mishra et al. (1994) J. Biol. Chem. 269, 7185-7191
Mishra et al. (1995) J. Biol. Chem. 270, 1602-1611
Mishra et al. (1998) Biochemistry, 37, 10313-10324
Morton R E (1999) Curr Opin Lipidol. 10, 321-327
Nagano et al. (2002) J. Lipid Res. 43, 1011-1018
Naito H K (1985) Ann. NY Acad. Sci, 454, 230-238
Nakagawa et al. (1985) J. Am. Chem. Soc. 107, 7087-7092
Ohnishi and Yokoyama (1993) Biochemistry, 32 (19), 5029-5035
Oka et al. (2000) Clin. Chem. 46, 1357-1364
Oka et al. (2000) J. Lipid Res. 41, 1651-1657
Oka et al. (2002) J. Lipid Res. 43, 1236-1243
Okamoto et al. (2000) Nature, 13, 406 (6792): 203-7
Oram and Lawn (2001) J. Lipid Res. 42, 1173-1179
Oram and Yokoyama (1997) J. Lipid Res. 37, 2473-2491
Packard and Shepherd (1997) Arteriosclerosis, Thromb, Vasc. Biology, 17, 3542-3556
Palgunachari et al. (1996) Arterioscler. Thromb. Vasc. Biol. 16, 328-338
Plump et al. (1997) Prc. Natl. Acad. Sci. USA, 91, 9607-9611
Ponsin et al. (1986a) J. Biol. Chem. 261, 9202-9205
Ponsin et al. (1986b) J. Clin. Invest. 77, 559-567
Pownall et al. (1980) Proc. Natl. Acad. Sci. USA, 77(6), 3154-3158
Pownall et al. (1985) Biochim. Biophys. Acta, 833, 456-462
Puchois et al. (1987) Atherosclerosis, 68, 35-40
Pussinen et al. (1997) J. Lipid Res. 38, 12-21
Pussinen et al. (1998) J. Lipid Res. 39, 152-161
Qin et al. (2000) J. Lipid Res. 41, 269-276
Ramsamy et al. (2000) J. Biol. Chem. 275, 33480-33486
Remaley et al. (1997) Arterioscler Thromb. Vasc. Biology, 17, 1813-1821
Reschly et al. (2002) J. Biol. Chem. 277, 9645-9654
Riemens et al. (1998) Atherosclerosis, 140, 71-79
Riemens et al. (1999) J. Lipid Res. 40, 1459-1466
Rinninger et al. (1998) J. Lipid Res. 39, 1335-1348
Ross R. (1993) Nature, 362, 801-809
Rothblat et al. (1999) J. Lipid Res. 40, 781-796
Rubin et al. (1991) Nature, 353, 265-267
Rubins, et al. (1999) N. Engl. J. Med. 341, 410-418
Santamarina-Fojo and Dugi (1994) Curr. Opin. Lipidol. 5, 117-125
Santamarina-Fojo et al. (2000) Curr. Opin. Lipidol. 11, 267-275
Schissel et al. (1996) J. Clin. Invest. 98, 1455-1464
Second Report of the Expert Panel (1994) Circulation, 89, 1329-1445
Segrest et al. (1983) Journal Biol Chem. 258, 2290-2295
Segrest et al. (1994) Advances in Protein Chem. 45, 303-369
Segrest et al. (2001) J. Lipid Res. 42, 1346-1367
Segrest J P (1974) FEBS Lett. 38, 247-253
Settasation et al. (2000) J. Biol. Chem. 276, 26898-26905
Shaefer E J (1994) Eur. J. Clin. Invest. 24, 441-443
Shatara et al. (2000) Can. J. Physiol. Pharmacol. 78, 367-371
Shepherd et al. (1995) N. Engl. J. Med. 333, 1301-1307
Sorci-Thomas et al. (1990) J. Biol. Chem. 265, 2665-2670
Sorci-Thomas et al. (2000) J. Biol. Chem. 275, 12156-12163
Sparks et al. (1992) J. Biol. Chem. 267, 25839-25847
Sparrow et al. (1981) In: “Peptides: Synthesis-Structure-Function,” Roch and Gross, Eds., Pierce Chem. Co., Rockford, Ill. 253-256
Sparrow et al. (2002) J. Biol. Chem. 277, 10021-10027
Srinivas et al. (1990) Virology, 176, 48-57
Stein and Stein (1999) Atherosclerosis, 144, 285-303
Steiner et al. (1987) Circulation, 75, 124-130
Steinmetz and Utermann (1985) J. Biol. Chem. 260, 2258-2264
Sviridov et al. (1996) Biochemistry, 35, 189-196
Sviridov et al. (2000) J. Biol. Chem. 275, 19707-19712
Sviridov et al. (2000) J. Lipid Res. 41, 1872-1882
Swinkels et al. (1989) Arteriosclerosis, 9, 604-613
Tall and Wang (2000) J. Clin. Invest. 106, 1205-1207
Tall et al. (2000) Arterioscler. Thromb, Vasc. Biol. 20, 1185-1188
Tall et al. (2001) J. Clin. Invest. 108, 1273-1275
Tall et al. (2001) J. Clin. Invest. 108, 1273-1275
Tangirala et al. (1999) Circulation, 100, 1816-1822
Temel et al. (2002) J. Biol. Chem. 277, 26565-26572
The BIP study group (2000) Circulation, 102, 21-27
The International Task Force for Prevention of Coronary Heart Disease (1998) Nutr Metab Cardiovasc Dis. 8, 205-271
Thuahnai et al. (2001) J. Biol. Chem. 276, 43801-43808
Tribble et al. (1992) Atherosclerosis, 93, 189-199
Trigatti et al. (1999) Proc. Natl. Acad. Sci. USA, 96, 9322-9327
Tu et al. (1993) J. Biol. Chem. 268, 23098-23105
Utermann et al. (1984) Eur. J. Biochem. 144, 325-331
Vakkilainen et al. (2002) J. Lipid Res. 43, 598-603
van Eck et al. (2002) Proc. Natl. Acad. Sci. U.S.A., 99, 6298-6303
Venkatachalapathi et al. (1993) Proteins, 15, 349-359
von Eckardstein A. (1996) Curr Opin Lipidol. 7, 308-319
von Eckardstein and Assmann (2000) Curr Opin Lipidol. 11, 627-637
von Eckardstein et al. (1995) Arterioscler. Thromb. Vasc. Biol. 15, 690-701
von Eckardstein et al. (1996) Biochim. Biophys. Acta, 1301, 255-262
von Eckardstein et al. (2001) Arterioscl. Thromb. Vasc. Biol. 21, 13
Webb et al. (2002) J. Lipid Res. 43, 1890-1898
Whayne et al. (1981) Atherosclerosis, 39, 411-424
Yamashita et al. (1991) Metabolism, 40, 756-763
Yamazaki et al. (1983) J. Biol. Chem. 258, 5847-5853
Zhong et al. (1994) Peptide Research, 7(2): 99-106 (1984) JAMA, 251, 365-374

What is claimed is:

1. A mediator of reverse cholesterol transport comprising a molecule comprising an acidic region, a lipophilic or aromatic region and a basic region, said molecule having a structure adapted to complex with HDL and/or low density lipoproteins-cholesterol (LDL) and thereby enhance reverse cholesterol transport.

2. The mediator of reverse cholesterol transport of claim 1, wherein said molecule has between 3 and 10 amino acid residues or analogs thereof, and comprises the sequence:
X1—X2—X3, wherein X1 is an acidic amino acid, X2 is a lipophilic or aromatic amino acid, and X3 is a basic amino acid, and wherein X1, X2 and X3 may be arranged in any sequential order;
wherein an amino terminal further comprises a first protecting group selected from the group consisting of an acetyl, phenylacetyl, pivolyl, 9-fluorenylmethyloxycarbonyl, 2-napthylic acid, nicotinic acid, a CH₃—(CH₂)ₙ—CO— where n ranges from 3 to 20, di-tert-butyl-4-hydroxy-phenyl, naphthyl, substituted naphthyl, f-MOC, biphenyl, substituted phenyl, substituted heterocycles, alkyl, aryl, substituted aryl, cycloalkyl, fused cycloalkyl, saturated heteroaryl, and substituted saturated heteroaryl; and
wherein a carboxy terminal further comprises a second protecting group selected from the group consisting of an amine, such as RNH₂ where R=H, di-tert-butyl-4-hydroxy-phenyl, naphthyl, substituted naphthyl, f-MOC, biphenyl, substituted phenyl, substituted heterocycles, alkyl, aryl, substituted aryl, cycloalkyl, fused cycloalkyl, saturated heteroaryl, and substituted saturated heteroaryl.

3. The mediator of reverse cholesterol transport of claim 2, wherein one or more of X1, X2 or X3 are D amino acid residues.

4. The mediator of reverse cholesterol transport of claim 2, wherein one or more of X1, X2 or X3 are modified synthetic or semisynthetic amino acids.

5. The mediator of reverse cholesterol transport of claim 4, wherein the modified synthetic or semi-synthetic amino acid is biphenylalanine.

6. A substantially pure amino acid-derived substance for treating and/or preventing hypercholesterolemia and/or atherosclerosis in a mammal, said substance having an amino and a carboxy terminal and comprising an L or D enantiomer of an acidic amino acid residue or derivative thereof, an L or D enantiomer of a lipophilic or aromatic amino acid residue or derivative thereof, and an L or D enantiomer of a basic amino acid residue or derivative thereof,
wherein the amino terminal further comprises a first protecting group selected from the group consisting of an acetyl, phenylacetyl, pivolyl, 9-fluorenylmethyloxycarbonyl, 2-napthylic acid, nicotinic acid, a CH₃—(CH₂)ₙ—CO— where n ranges from 3 to 20, di-tert-butyl-4-hydroxy-phenyl, naphthyl, substituted naphthyl, f-MOC, biphenyl, substituted phenyl, substituted heterocycles, alkyl, aryl, substituted aryl, cycloalkyl, fused cycloalkyl, saturated heteroaryl, substituted saturated heteroaryl, and the like;
wherein the carboxy terminal further comprises a second protecting group selected from the group consisting of an amine, such as RNH₂ where R=H, di-tert-butyl-4-hydroxy-phenyl, naphthyl, substituted naphthyl, f-MOC, biphenyl, substituted phenyl, substituted heterocycles, alkyl, aryl, substituted aryl, cycloalkyl, fused cycloalkyl, saturated heteroaryl, substituted saturated heteroaryl, and the like; and
wherein said substance has at least one of the following properties: (1) it mimicks ApoA-I binding to LDL and HDL, (2) it binds preferentially to liver, (3) it enhances LDL uptake by liver LDL-receptors, (4) it lower the levels of LDL, IDL, and VLDL cholesterol, (5) it increases the levels of HDL cholesterol, and (6) it enhances plasma lipoprotein profiles.

7. A composition suitable for oral administration that ameliorates or prevents a symptom of hypercholesterolemia, wherein said composition comprises an amino acid-derived molecule having an acidic region, a lipophilic or aromatic region and a basic region, and wherein said amino acid-derived molecule has a first protecting group attached to an amino terminal and a second protecting group attached to a carboxyl terminal, and wherein said amino acid-derived molecule optionally comprises at least one D amino acid residue.

8. The composition of claim 7, which includes at least one D amino acid residue

9. A peptide mediator of RCT, comprising the sequence:
Xₐ—X_{b}—(X1—X²—X³)—X_{c}—X_{d},
wherein
Xₐ is an acylated amino acid residue;
X_{b} is any 0-10 amino acid residues;
X1—X2—X3 are amino acid residues or derivatives thereof selected independently from an acidic amino acid residue, a lipophilic amino acid residue, and a basic amino acid residue or derivative thereof, with the proviso that one of said X1, X2 or X3 is an acidic residue, one of said X1, X2 or X3 is a lipophilic residue and one of said X1, X2 or X3 is a basic residue;
X_{c} is any 0-10 amino acid residues;
X_{d} is an amidated amino acid residue; and
wherein the peptide mediator has 15 or fewer amino acid residues and optionally comprises at least one D amino acid residue.

10. An RCT mediator, comprising a compound selected from the group consisting of the synthetic compounds 1-96 of Table 5.

11. An RCT mediator, comprising a compound selected from the group consisting of SEQ ID NOS: 1 and 107-117.

12. An RCT mediator, comprising a compound selected from the group consisting of SEQ ID NOS: 1, 26-36, 42, 45-47, 56-58, 68-70, 72-74, 76, 80, 81, 83-90 and 92-95.

13. A pharmaceutical composition comprising SEQ ID NO: 1.

14. A pharmaceutical composition comprising SEQ ID NO: 113.

15. A pharmaceutical composition comprising SEQ ID NO: 34.

16. A pharmaceutical composition comprising SEQ ID NO: 86.

17. A pharmaceutical composition comprising SEQ ID NO: 91.

18. A pharmaceutical composition comprising SEQ ID NO: 96.

19. A pharmaceutical composition comprising SEQ ID NO: 145.

20. A pharmaceutical composition comprising SEQ ID NO: 146.

21. A pharmaceutical composition comprising SEQ ID NO: 118.

22. A method for treating or preventing hypercholesterolemia and/or atherosclerosis, comprising administering to a mammal in need thereof an amount of a composition selected from the group consisting of SEQ ID NOS: 1-176 (Table 3) and synthetic compounds 1-96 (Table 5), wherein said amount is sufficient to enhance RCT and/or cause regression of existing atherosclerotic lesions or reduce formation of said lesions.

23. A method for treating or preventing hypercholesterolemia and/or atherosclerosis, comprising administering to a mammal in need thereof an amount of a composition selected from the group consisting of SEQ ID NOS: 1, 113, 34, 86, 91, 96, 145, 146, and 118, wherein said amount is sufficient to enhance RCT and/or cause regression of existing atherosclerotic lesions or reduce formation of said lesions.

24. The method of claim 22, wherein administering is via an oral route.

25. The method of claim 22, wherein administering is combined with administration of a bile acid-binding resin, niacin, a statin, or a combination thereof.

26. An in vitro screening method for identifying test compounds that are likely to enhance reverse cholesterol transport in vivo, comprising:
measuring cholesterol accumulation in liver cells in vitro in the presence and absence of the test compounds;
measuring cholesterol accumulation and/or efflux in AcLDL-loaded macrophages in vitro in the presence and absence of test compounds; and
identifying test compounds that enhance cholesterol accumulation in liver cells and reduce cholesterol levels in macrophages.

27. The in vitro screening method of claim 26, further comprising measuring cholesterol levels in OxLDL-loaded vascular smooth muscle cells in vitro in the presence and absence of test compounds, and wherein the step of identifying test compounds further comprises identifying compounds that enhance cholesterol accumulation in liver cells and reduce cholesterol levels in macrophages and/or reduce cholesterol levels in vascular smooth muscle cells.

28. The in vitro screening method of claim 26, wherein said liver cells are human HepG2 hepatoma cells.

29. The in vitro screening method of claim 26, wherein said macrophages are human THP-1 cells.

30. The in vitro screening method of claim 27, wherein said vascular smooth muscle cells are primary aortic smooth muscle cells.

31. An in vitro screening method for identifying test compounds that are likely to enhance reverse cholesterol transport in vivo, comprising:
measuring cholesterol accumulation in liver cells in vitro in the presence and absence of the test compounds;
measuring cholesterol levels in AcLDL-loaded vascular smooth muscle cells in vitro in the presence and absence of test compounds; and
identifying test compounds that enhance cholesterol accumulation in liver cells and reduce cholesterol levels in vascular smooth muscle cells.
